(19) United States
(12) Patent Application Publication
(10) Pub. No.: US 2023/0203178 A1
(43) Pub. Date: Jun. 29, 2023

(54) CHIMERIC ANTIGEN RECEPTOR CAR OR CAR CONSTRUCT TARGETING BCMA AND CD19 AND APPLICATION THEREOF

(71) Applicant: Sichuan Kelun-Biotech Biopharmaceutical Co., Ltd., Chengdu (CN)

(72) Inventors: Jianhui CHANG, Chengdu (CN); Yanlin ZHU, Chengdu (CN); Guangjun JING, Suzhou (CN); Jingyi WANG, Chengdu (CN); Jiangman WANG, Chengdu (CN); Zhenzhen CAI, Chengdu (CN); Panyan HOU, Chengdu (CN); Liang XIAO, Chengdu (CN); Tongtong XUE, Chengdu (CN); Xiaodong WANG, Chengdu (CN)

(21) Appl. No.: 18/008,315

(22) PCT Filed: Jun. 25, 2021
(86) PCT No.: PCT/CN2021/102417

§ 371 Date: Dec. 5, 2022

(30) Foreign Application Priority Data
Jul. 6, 2020 (CN) 202010644219.0

(51) Int. Cl. C07K 16/28; C07K 14/705; C07K 14/725; C12N 5/0783; A61K 45/06; A61K 39/395; A61K 38/17; A61K 35/17; A61P 35/00

(52) U.S. Cl. CPC C07K 16/2878; C07K 16/2803; C07K 14/70517; C07K 14/70521; C07K 14/7051; C07K 14/70578; C12N 5/0636; A61K 45/06; A61K 39/3955; A61K 38/1774; A61K 38/177; A61K 35/17; A61P 35/00; A61K 2039/505

(57) ABSTRACT

Provided are a chimeric antigen receptor (CAR) or CAR construct containing antibodies against BCMA and CD19, a nucleic acid molecule encoding the CAR or CAR construct, a modified immune cell, and a method for preparing the immune cell. The CAR or CAR construct and the modified immune cell are used for the prevention and/or treatment of B cell related conditions (e.g., B cell and plasma cell related malignant tumors or autoimmune diseases (such as systemic lupus erythematosus)), and can effectively avoid target escape and prevent the recurrence of multiple myeloma.

### FIELD OF THE INVENTION

The present invention relates to the field of medicine and biology, in particular, the present invention relates to anti-BCMA and CD19 antibodies and antigen-binding fragments thereof, and chimeric antigen receptors (CARs) or CAR constructs comprising the same, and further relates to CARs or CAR constructs targeting BCMA and CD19. The present invention also relates to nucleic acid molecules encoding such CARs or CAR constructs targeting BCMA and CD19, engineered immune cells comprising such CARs or CAR constructs targeting BCMA and CD19, and a method for preparing such engineered immune cells. The present invention also relates to use of such CARs or CAR constructs targeting BCMA and CD19 and engineered immune cells for the prevention and/or treatment of B cell-related diseases (e.g., B cell- and plasma cell-related malignancies or autoimmune diseases (e.g., systemic lupus erythematosus, etc.) and a method for preventing and/or treating B cell-related diseases.

### BACKGROUND OF THE INVENTION

B-cell maturation antigen (BCMA) is a member of the tumor necrosis factor (TNF) receptor superfamily (also known as tumor necrosis factor receptor superfamily member 17 (TNFRF17), CD269, etc.), which contains 184 amino acids and is a type I transmembrane protein. BCMA is mainly expressed in late-stage mature B cell subsets, such as plasma cells. It is neither expressed in hematopoietic stem cells and early-stage B cells, nor expressed in non-hematopoietic tissues. The ligands of BCMA are B-cell activating factor (BAFF) and proliferation-inducing ligand (APRIL). After the ligand B lymphocyte stimulator (BLyS) binding to BCMA, it activates the downstream NF-kappaB and MAPK8/JNK pathway of BCMA, and promotes the proliferation and differentiation of B cells and promotes the production of antibodies. After the ligand APRIL binds to BCMA, it can promote the growth of multiple myeloma (MM) cells and generate an immunosuppressive microenvironment in the bone marrow. In patients with multiple myeloma, elevated serum concentration of free BCMA compete for binding to BAFF, resulting in impaired plasma cell activation. Therefore, BCMA plays an important role in MM disease progression. Recently, data from multiple clinical trials of chimeric antigen receptor T cell (CAR-T) therapy developed with BCMA as the target show that BCMA-targeted CAR-T therapy shows good curative effect in the treatment of MM.

CD19 is a 95 kDa glycoprotein on the surface of B cells, which is expressed from the early stage of B cell development until the stage of differentiation into plasma cells. CD19 is a member of the immunoglobulin (Ig) superfamily. As one of the components of the B cell surface signal transduction complex, CD19 is involved in regulating the signal transduction of B cell receptors. CD19 is a potential target for the treatment of tumors associated with B lymphocytes and a hot spot in CAR research. The expression of CD19 is limited to normal and malignant B cells, and it is a widely accepted CAR target for safety testing. Chimeric antigen receptor gene-modified T cells targeting CD19 molecules (CD19 CAR-T) have achieved significant efficacy in the treatment of multiple, refractory B-cell acute lymphoblastic leukemia. Systemic lupus erythematosus (SLE) is a typical autoimmune disease with complex pathogenesis, involving many important organs, such as heart, brain and kidney, and its pathogeny has not yet been determined. The current therapeutic regimens have relatively serious side effects, must be used for a long time to control the progression of the disease, and cannot achieve radical cure. Glucocorticoids are still the first-line drug for SLE, which requires long-term medication and cannot achieve radical cure. Other drugs have limited efficacy or have serious side effects. CD19 CAR-T preclinical data show that it can effectively cure lupus mice, and has entered the clinical trial (Gene Chem Co., Ltd, NCT03030976). UniSR University in Italy carried out an early study of BCMA CAR-T in the treatment of systemic lupus erythematosus.

However, similar to the recurrence in the treatment of B-cell acute lymphoblastic leukemia with CD19 CAR, the recurrence of BCMA-positive/BCMA-negative tumor cells were also observed in the treatment of MM with BCMA CAR-T. The recurrence of BCMA-positive cells indicates that BCMA CAR-T lacks treatment persistence, while the recurrence of BCMA-negative cells indicates that the target escape of BCMA antigens occurred through selective pressure. Meanwhile, clinical trials have shown that MM tumor stem cells are CD19 positive. In addition, immunological and molecular biological studies suggest that MM originates from the malignant transformation of pre-B cells, or from the malignant transformation of hematopoietic precursor cells earlier than pre-B cells. Therefore, the elimination of malignant B cells originated from early stage will greatly improve the therapeutic effect of MM and delay the recurrence, and hence CD19 is a potential therapeutic target for the elimination of relapsed, refractory or drug-resistant MM diseases. Therefore, it is particularly important to find a chimeric antigen receptor CAR or CAR construct that can effectively improve the therapeutic effect of CAR-T.

### CONTENT OF THE INVENTION

In the present application, the inventors developed human antibodies with excellent properties capable of specifically recognizing/binding BCMA and CD19, respectively. On this basis, the inventor has made a lot of creative work, further designed and constructed chimeric antigen receptor CAR or CAR constructs with different structures targeting BCMA and CD19, and obtained CAR or CAR constructs that can target BCMA and CD19. The CAR-modified immune cells of the present invention are capable of directing the specificity and reactivity of immune effector cell to BCMA- and CD19-expressing cells (e.g., malignant B cells, plasma cells, and plasmacytoid B cells) in a non-MHC-restricted manner to allow them to be eliminated, and have similar functional properties compared to BCMA-targeting CARs and CD19-targeting CARs. Therefore, the immune cells modified with the BCMA- and CD19-targeting CAR or CAR constructs of the present invention have potency in the prevention and/or treatment of B cell-related conditions (e.g., B cell- and plasma cell-related malignancies or autoimmune diseases (e.g., systemic lupus erythematosus), etc.), can effectively avoid target escape, and prevent the recurrence of MM, and thus have great clinical values. Therefore, the BCMA and CD19 CAR-T constructed herein not only can improve the efficacy of treatment of MM, but also provide an optional treatment for systemic lupus erythematosus.

Antibodies or Antigen-Binding Fragments Thereof

In one aspect, the present invention provides a bispecific antibody or antigen-binding fragment thereof that targets BCMA and CD19, characterized in that the bispecific antibody or antigen-binding fragment thereof comprises a first antibody or antigen-binding fragment thereof targeting BCMA and a second antibody or antigen-binding fragment thereof targeting CD19, the first antibody or antigen-binding fragment thereof targeting BCMA comprises a first heavy chain variable region (VH) and/or a first light chain variable region (VL), the first VH and/or the first VL form a BCMA-binding site, and the second antibody or antigen-binding fragment thereof targeting CD19 comprises a second heavy chain variable region (VH) and/or a second light chain variable region (VL), the second VH and/or the second VL form a CD19-binding site, wherein,

the first VH comprises: a first VH CDR1 comprising an amino acid sequence as set forth in SEQ ID NO: 5 or a sequence having a substitution, deletion or addition of one or several amino acids (e.g., a substitution, deletion or addition of 1, 2 or 3 amino acids) as compared thereto; a first VH CDR2 comprising an amino acid sequence as set forth in SEQ ID NO: 6 or a sequence having a substitution, deletion or addition of one or several amino acids (e.g., a substitution, deletion or addition of 1, 2 or 3 amino acids) as compared thereto; and a first VH CDR3 comprising an amino acid sequence as set forth in SEQ ID NO: 7 or a sequence having a substitution, deletion or addition of one or several amino acids (e.g., a substitution, deletion or addition of 1, 2 or 3 amino acids) as compared thereto;

the first VL comprises: a first VL CDR1 comprising an amino acid sequence as set forth in SEQ ID NO: 8 or a sequence having a substitution, deletion or addition of one or several amino acids (e.g., a substitution, deletion or addition of 1, 2 or 3 amino acids) as compared thereto; a first VL CDR2 comprising an amino acid sequence as set forth in SEQ ID NO: 9 or a sequence having a substitution, deletion or addition of one or several amino acids (e.g., a substitution, deletion or addition of 1, 2 or 3 amino acids) as compared thereto; and a first VL CDR3 comprising an amino acid sequence as set forth in SEQ ID NO: 10 or a sequence having a substitution, deletion or addition of one or several amino acids (e.g., a substitution, deletion or addition of 1, 2 or 3 amino acids) as compared thereto; preferably, the substitution is a conservative substitution.

In certain embodiments, the first VH comprises the sequence as set forth in SEQ ID NO: 1 or a variant thereof; the first VL comprises the sequence as set forth in SEQ ID NO: 2 or a variant thereof; wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4, or 5 amino acids) as compared to the sequence from which it is derived.

Further, the second VH of the second antibody or antigen-binding fragment thereof targeting CD19, comprises: a second VH CDR1 comprising an amino acid sequence as set forth in SEQ ID NO: 11 or a sequence having a substitution, deletion or addition of one or several amino acids (e.g., a substitution, deletion or addition of 1, 2 or 3 amino acids) as compared thereto; a second VH CDR2 comprising an amino acid sequence as set forth in SEQ ID NO: 12 or a sequence having a substitution, deletion or addition of one or several amino acids (e.g., a substitution, deletion or addition of 1, 2 or 3 amino acids) as compared thereto; and a second VH CDR3 comprising an amino acid sequence as set forth in SEQ ID NO: 13 or a sequence having a substitution, deletion or addition of one or several amino acids (e.g., a substitution, deletion or addition of 1, 2 or 3 amino acids) as compared thereto;

the second VL comprises: a second VL CDR1 comprising an amino acid sequence as set forth in SEQ ID NO: 14 or a sequence having a substitution, deletion or addition of one or several amino acids (e.g., a substitution, deletion or addition of 1, 2 or 3 amino acids) as compared thereto; a second VL CDR2 comprising an amino acid sequence as set forth in SEQ ID NO: 15 or 53 or a sequence having a substitution, deletion or addition of one or several amino acids (e.g., a substitution, deletion or addition of 1, 2 or 3 amino acids) as compared thereto; and a second VL CDR3 comprising an amino acid sequence as set forth in SEQ ID NO: 16 or a sequence having a substitution, deletion or addition of one or several amino acids (e.g., a substitution, deletion or addition of 1, 2 or 3 amino acids) as compared thereto; preferably, the substitution is a conservative substitution.

In certain embodiments, the second VH comprises the sequence as set forth in SEQ ID NO: 3 or 76 or a variant thereof; the second VL comprises the sequence as set forth in SEQ ID NO: 4 or 77 or a variant thereof; wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4, or 5 amino acids) as compared to the sequence from which it is derived.

In certain embodiments, the bispecific antibody or antigen-binding fragment thereof comprises the following domains in sequence from N-terminal to C-terminal: “first VH, first VL, second VH, second VL”; “second VH, second VL, first VH, first VL”; “first VL, first VH, second VL, second VH”; “second VL, second VH, first VL, first VH”; “first VH, first VL, second VL, second VH”; “second VH, second VL, first VL, first VH”; “first VL, first VH, second VH, second VL”; “second VL, second VH, first VH, first VL”; “first VL, second VL, second VH, first VH”; “second VL, first VL, first VH, second VH”; “first VH, second VL, second VH, first VL”; “second VH, first VL, first VH, second VL”; “first VL, second VH, second VL, first VH”; “second VL, first VH, first VL, second VH”; “first VH, second VH, second VL, first VL”; or “second VH, first VH, first VL, second VL”. Any of the above-mentioned adjacent variable regions are independently connected by a linker, and the linker between the adjacent variable regions may be the same or different. In certain embodiments, the linker is a polypeptide having a sequence as set forth in (GGGGS)x1 or (EAAAK)x2, wherein x1 and x2 are independently selected from integers from 1 to 6; in certain embodiments, the linker is a polypeptide containing the sequence as set forth in SEQ ID NO:68. In certain embodiments, the linker is selected from a polypeptide of the sequence as set forth in SEQ ID NO: 17, 18, 19, 20 or 68.

In certain embodiments, the present invention provides an antibody or antigen-binding fragment thereof that targets BCMA, which comprises a heavy chain and a light chain, the heavy chain comprises a first VH of the sequence as set forth in SEQ ID NO: 1 or a variant thereof, the light chain comprises a first VL of the sequence as set forth in SEQ ID NO: 2 or a variant thereof;

wherein the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the present invention provides an antibody or antigen-binding fragment thereof that targets CD19, which comprises a heavy chain and a light chain, the heavy chain comprises a second VH of the sequence as set forth in SEQ ID NO: 3 or 76 or a variant thereof, the light chain comprises a second VL of the sequence as set forth in SEQ ID NO: 4 or 77 or a variant thereof;

wherein the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the said substitution is a conservative substitution.

In certain embodiments, the bispecific antibody or antigen-binding fragment thereof of the present invention further comprises a constant region sequence or variant thereof derived from a mammalian (e.g., murine or human) immunoglobulin, and the variant has a substitution, deletion or addition of one or more amino acids as compared to the wild-type sequence from which it is derived. In certain embodiments, the variant has a conservative substitution of one or more amino acids compared to the wild-type sequence from which it is derived.

In certain embodiments, the bispecific antibody or antigen-binding fragment thereof of the present invention comprises a heavy chain constant region (CH) of a human immunoglobulin or variant thereof, and the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared to the wild-type sequence from which it is derived; and/or,

the bispecific antibody or antigen-binding fragment thereof of the present invention comprises a light chain constant region (CL) of a human immunoglobulin or variant thereof, and the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared to the wild-type sequence from which it is derived.

In certain embodiments, the bispecific antibody or antigen-binding fragment thereof of the present invention comprises a heavy chain constant region (CH) of a human immunoglobulin or variant thereof and the variant has a conservative substitution of up to 20 amino acids (e.g., a conservative substitution of up to 15, up to 10, or up to 5 amino acids; for example, a conservative substitution of 1, 2, 3, 4 or 5 amino acids) as compared to the wild-type sequence from which it is derived; and/or,

the bispecific antibody or antigen-binding fragment thereof of the present invention comprises a light chain constant region (CL) of a human immunoglobulin or variant thereof, and the variant has a conservative substitution of up to 20 amino acids (e.g., a conservative substitution of up to 15, up to 10, or up to 5 amino acids; for example, a conservative substitution of 1, 2, 3, 4 or 5 amino acids) as compared to the wild-type sequence from which it is derived.

In certain embodiments, the bispecific antibody or antigen-binding fragment thereof of the present invention comprises a heavy chain constant region (CH) of a murine immunoglobulin or variant thereof, and the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared to the wild-type sequence from which it is derived; and/or,

the bispecific antibody or antigen-binding fragment thereof of the present invention comprises a light chain constant region (CL) of a murine immunoglobulin or variant thereof, and the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared to the wild-type sequence from which it is derived.

In certain embodiments, the bispecific antibody or antigen-binding fragment thereof of the present invention comprises a heavy chain constant region (CH) of a murine immunoglobulin or variant thereof, and the variant has a conservative substitution of up to 20 amino acids (e.g., a conservative substitution of up to 15, up to 10, or up to 5 amino acids; for example, a conservative substitution of 1, 2, 3, 4 or 5 amino acids) as compared to the wild-type sequence from which it is derived; and/or,

the bispecific antibody or antigen-binding fragment thereof of the present invention comprises a light chain constant region (CL) of a murine immunoglobulin or variant thereof, and the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared to the wild-type sequence from which it is derived.

In certain embodiments, the heavy chain constant region is an IgG, IgM, IgE, IgD or IgA heavy chain constant region. In certain embodiments, the heavy chain constant region is an IgG heavy chain constant region, for example, an IgG1, IgG2, IgG3 or IgG4 heavy chain constant region. In certain embodiments, the heavy chain constant region is a human IgG1 or IgG4 heavy chain constant region.

In certain embodiments, the light chain constant region is a κ or λ light chain constant region. In certain preferred embodiments, the light chain constant region is a human κ light chain constant region.

In certain embodiments, the first antibody or antigen-binding fragment thereof targeting BCMA or the second antibody or antigen-binding fragment thereof targeting CD19 is each independently selected from the group consisting of camelid Ig, IgNAR, Fab fragment, Fab′ fragment, F(ab′)₂ fragment, F(ab′)₃ fragment, Fv, single chain antibody (e.g., scFv, di-scFv, (scFv)₂), minibody, bifunctional antibody, trifunctional antibody, tetrafunctional antibody, disulfide-stabilized Fv protein (“dsFv”) and single domain antibody (sdAb, nanobody), chimeric antibody, humanized antibody, single domain antibody, bispecific antibody or multispecific antibody.

In certain embodiments, the first antibody or antigen-binding fragment thereof targeting BCMA or the second antibody or antigen-binding fragment thereof targeting CD19 is an scFv; in certain embodiments, the scFv targeting BCMA comprises a first VL as set forth in SEQ ID NO: 2, a linker as set forth in SEQ ID NO: 17, 18, 19, 20 or 68, a first VH as set forth in SEQ ID NO: 1; the scFv targeting CD19 comprises a second VL as set forth in SEQ ID NO: 4 or 77, a linker as set forth in SEQ ID NO: 17, 18, 19, 20 or 68, and a second VH as set forth in SEQ ID NO: 3 or 76.

In certain embodiments, the present invention provides a scFv targeting BCMA, which comprises a sequence as set forth in SEQ ID NO: 25 or variant thereof;

wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the present invention provides a scFv targeting CD19, which comprises a sequence as set forth in SEQ ID NO: 26 or variant thereof;

wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the present invention provides a scFv targeting BCMA, which comprises a sequence as set forth in SEQ ID NO: 27 or variant thereof;

wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the present invention provides a scFv targeting CD19, which comprises a sequence as set forth in SEQ ID NO: 28 or variant thereof;

wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the bispecific antibody or antigen-binding fragment thereof of the present invention has a feature of being capable of specifically binding to an antigen or receptor, such as CD20, CD22, CD33, CD123, or CD138, that can induce an immune response.

Preparation of Antibody

The antibody of the present invention can be prepared by various methods known in the art, such as by genetic engineering recombinant techniques. For example, a DNA molecule encoding the heavy and light chain genes of the antibody of the present invention can be obtained by chemical synthesis or PCR amplification; the resulting DNA molecule is inserted into an expression vector, and then transfected into a host cell. Then, the transfected host cell is cultured under a specific condition and expresses the antibodies of the present invention.

The antigen-binding fragment of the present invention can be obtained by hydrolysis of an intact antibody molecule (see: Morimoto et al., J. Biochem. Biophys. Methods 24:107-117 (1992) and Brennan et al., Science 229:81 (1985)). Alternatively, the antigen-binding fragment can also be produced directly from a recombinant host cell (reviewed in Hudson, Curr. Opin. Immunol. 11:548-557 (1999); Little et al., Immunol. Today, 21:364-370 (2000)). For example, a Fab′ fragment can be obtained directly from a host cell; a F(ab′)₂ fragment can be formed by chemically coupling Fab′ fragments (Carter et al., Bio/Technology, 10: 163-167 (1992)). In addition, a Fv, Fab or F(ab′)₂ fragment can also be directly isolated from a culture medium of recombinant host cells. Other techniques for preparing these antigen-binding fragments are well known to those of ordinary skill in the art.

Chimeric Antigen Receptor (CAR)

The antibody or antigen-binding fragment thereof of the present invention can be used to construct a chimeric antigen receptor (CAR). The features of the CAR of the present invention comprises ability of recognizing BCMA and CD19 in non-MHC-restricted manner, which confers an ability of recognizing a cell expressing BCMA and CD19 independent of antigen processing and presentation on an immune cell (e.g., T cell, NK cell, monocyte, macrophage or dendritic cell) that expresses the CAR.

In an embodiment of the present invention, the present invention provides a chimeric antigen receptor (CAR), wherein each CAR is capable of binding two antigens (e.g., BCMA and CD19) simultaneously. These CARs are bispecific against BCMA and CD19. The phrase “bispecific” as used herein with respect to CAR means that one single CAR is capable of specifically binding and immunologically recognizing two different antigens, such that the binding of the CAR to at least one of the two antigens induces an immunity response. Examples of such CARs herein include, for example, tandem CAR (TanCAR). Examples of such CARs herein include at least TanCARs 01-06, 08, 10 as described below.

Accordingly, in another aspect, the present invention provides a chimeric antigen receptor (CAR) targeting BCMA and CD19, which comprises an antigen-binding domain, a spacer domain, a transmembrane domain, and an intracellular signaling domain, and the antigen-binding domain comprises the bispecific antibody or antigen-binding fragment thereof of any one of the preceding embodiments.

In certain embodiments, the chimeric antigen receptor (CAR) targeting BCMA and CD19 comprises the following domains in sequence from N-terminal to C-terminal:

(1) the first VH, the first VL, the second VH, the second VL, the spacer domain, the transmembrane domain, and the intracellular signaling domain;

(2) the second VH, the second VL, the first VH, the first VL, the spacer domain, the transmembrane domain, and the intracellular signaling domain;

(3) the first VL, the first VH, the second VL, the second VH, the spacer domain, the transmembrane domain, and the intracellular signaling domain;

(4) the second VL, the second VH, the first VL, the first VH, the spacer domain, the transmembrane domain, and the intracellular signaling domain;

(5) the first VH, the first VL, the second VL, the second VH, the spacer domain, the transmembrane domain, and the intracellular signaling domain;

(6) the second VH, the second VL, the first VL, the first VH, the spacer domain, the transmembrane domain, and the intracellular signaling domain;

(7) the first VL, the first VH, the second VH, the second VL, the spacer domain, the transmembrane domain, and the intracellular signaling domain;

(8) the second VL, the second VH, the first VH, the first VL, the spacer domain, the transmembrane domain, and the intracellular signaling domain;

(9) the first VL, the second VL, the second VH, the first VH, the spacer domain, the transmembrane domain, and the intracellular signaling domain;

(10) the second VL, the first VL, the first VH, the second VH, the spacer domain, the transmembrane domain, and the intracellular signaling domain;

(11) the first VH, the second VL, the second VH, the first VL, the spacer domain, the transmembrane domain, and the intracellular signaling domain;

(12) the second VH, the first VL, the first VH, the second VL, the spacer domain, the transmembrane domain, and the intracellular signaling domain;

(13) the first VL, the second VH, the second VL, the first VH, the spacer domain, the transmembrane domain, and the intracellular signaling domain;

(14) the second VL, the first VH, the first VL, the second VH, the spacer domain, the transmembrane domain, and the intracellular signaling domain;

(15) the first VH, the second VH, the second VL, the first VL, the spacer domain, the transmembrane domain, and the intracellular signaling domain; or

(16) the second VH, the first VH, the first VL, the second VL, the spacer domain, the transmembrane domain and the intracellular signaling domain; optionally, wherein each VH and/or VL is connected by a linker;

in any one of items (1) to (16), any adjacent variable regions are each independently connected by a linker; preferably, the linker between any adjacent variable regions is independently selected from: a polypeptide having a sequence as set forth in (GGGGS)x1 or (EAAAK)x2 (x1 and x2 are independently selected from integers from 1 to 6) or a polypeptide comprising a sequence as set forth in SEQ ID NO: 68; preferably, in any one of items (1) to (16), the linker between adjacent variable regions may be the same or different.

Antigen-Binding Domain of CAR

The antigen-binding domain contained in the CAR of the present invention confers the ability of recognizing BCMA and CD19 on the CAR.

In certain embodiments, the antigen-binding domain includes, but is not limited to, camelid Ig, IgNAR, Fab fragment, Fab′ fragment, F(ab′)₂ fragment, F(ab′)₃ fragment, Fv, single chain antibody (e.g., scFv, di-scFv, (scFv)₂), minibody, bifunctional antibody, trifunctional antibody, tetrafunctional antibody, disulfide-stabilized Fv protein (“dsFv”) and single domain antibody (sdAb, nanobody).

In certain embodiments, the antigen-binding domain typically comprises at least one variable region. The variable region can be of any size or composited of any amino acids, and generally comprises at least one CDR which is adjacent to one or more framework sequences or in one framework with one or more framework sequences. In the antigen-binding domain comprising heavy chain variable region (VH) and/or light chain variable region (VL), the VH and VL may be positioned relative to each other in any suitable arrangement, for example, VH-VH, VH-VL, VL-VL or VL-VH. Alternatively, the antigen-binding domain may comprise a VH or VL domain. The antigen-binding domain can form any engineering possible structure, such as a single chain antibody (e.g., scFv, di-scFv, (scFv)₂) comprising VH-VL, VH-VH, VL-VL, VL-VH, or bifunctional antibody, trifunctional antibody, tetrafunctional antibody, disulfide-stabilized Fv protein, camelid Ig, IgNAR, etc. Any adjacent variable regions are independently connected by a linker; preferably, the linker between any adjacent variable regions is independently selected from the sequence as set forth in SEQ ID NO: 17, 18, 19, 20 or 68.

In certain embodiments, the present invention provides a chimeric antigen receptor capable of targeting BCMA and CD19, which comprises an antigen-binding domain, a spacer domain, a transmembrane domain, and an intracellular signaling domain, the antigen-binding domain comprises the aforementioned first antibody or antigen-binding fragment thereof targeting BCMA and the aforementioned second antibody or antigen-binding fragment thereof targeting CD19, the first antibody or antigen-binding fragment thereof targeting BCMA comprises a first heavy chain variable region (VH) and/or a first light chain variable region (VL), the first VH and/or the first VL form a BCMA-binding site; and the second antibody or antigen-binding fragment thereof targeting CD19 comprises a second heavy chain variable region (VH) and/or a second light chain variable region (VL), and the second VH and/or the second VL form a CD19-binding site,

the first VH comprises: a first VH CDR1 comprising an amino acid sequence as set forth in SEQ ID NO: 5 or a sequence having a substitution, deletion or addition of one or several amino acids (e.g., a substitution, deletion or addition of 1, 2 or 3 amino acids) as compared thereto; a first VH CDR2 comprising an amino acid sequence as set forth in SEQ ID NO: 6 or a sequence having a substitution, deletion or addition of one or several amino acids (e.g., a substitution, deletion or addition of 1, 2 or 3 amino acids) as compared thereto; and a first VH CDR3 comprising an amino acid sequence as set forth in SEQ ID NO: 7 or a sequence having a substitution, deletion or addition of one or several amino acids (e.g., a substitution, deletion or addition of 1, 2 or 3 amino acids) as compared thereto;

the first VL comprises: a first VL CDR1 comprising an amino acid sequence as set forth in SEQ ID NO: 8 or a sequence having a substitution, deletion or addition of one or several amino acids (e.g., a substitution, deletion or addition of 1, 2 or 3 amino acids) as compared thereto; a first VL CDR2 comprising an amino acid sequence as set forth in SEQ ID NO: 9 or a sequence having a substitution, deletion or addition of one or several amino acids (e.g., a substitution, deletion or addition of 1, 2 or 3 amino acids) as compared thereto; and a first VL CDR3 comprising an amino acid sequence as set forth in SEQ ID NO: 10 or a sequence having a substitution, deletion or addition of one or several amino acids (e.g., a substitution, deletion or addition of 1, 2 or 3 amino acids) as compared thereto;

the second VH comprises: a second VH CDR1 comprising an amino acid sequence as set forth in SEQ ID NO: 11 or a sequence having a substitution, deletion or addition of one or several amino acids (e.g., a substitution, deletion or addition of 1, 2 or 3 amino acids) as compared thereto; a second VH CDR2 comprising an amino acid sequence as set forth in SEQ ID NO: 12 or a sequence having a substitution, deletion or addition of one or several amino acids (e.g., a substitution, deletion or addition of 1, 2 or 3 amino acids) as compared thereto; and a second VH CDR3 comprising an amino acid sequence as set forth in SEQ ID NO: 13 or a sequence having a substitution, deletion or addition of one or several amino acids (e.g., a substitution, deletion or addition of 1, 2 or 3 amino acids) as compared thereto;

the second VL comprises: a second VL CDR1 comprising an amino acid sequence as set forth in SEQ ID NO: 14 or a sequence having a substitution, deletion or addition of one or several amino acids (e.g., a substitution, deletion or addition of 1, 2 or 3 amino acids) as compared thereto; a second VL CDR2 comprising an amino acid sequence as set forth in SEQ ID NO: 15 or 53 or a sequence having a substitution, deletion or addition of one or several amino acids (e.g., a substitution, deletion or addition of 1, 2 or 3 amino acids) as compared thereto; and a second VL CDR3 comprising an amino acid sequence as set forth in SEQ ID NO: 16 or a sequence having a substitution, deletion or addition of one or several amino acids (e.g., a substitution, deletion or addition of 1, 2 or 3 amino acids) as compared thereto.

In some embodiments, the substitution is a conservative substitution.

In certain embodiments, the CDR1, CDR2 and CDR3 contained in the heavy chain variable region (VH), and/or the CDR1, CDR2 and CDR3 contained in the light chain variable region (VL) are defined by Kabat, Chothia or IMGT numbering system. In certain exemplary embodiments, the CDR1, CDR2 and CDR3 contained in the heavy chain variable region (VH), and/or the CDR1, CDR2 and CDR3 contained in the light chain variable region (VL) are defined by Chothia numbering system.

In certain embodiments, the present invention provides a chimeric antigen receptor targeting BCMA and CD19, which comprises an antigen-binding domain, a spacer domain, a transmembrane domain, and an intracellular signaling domain, wherein the antigen-binding domain comprises the follows in sequence from the N-terminal to the C-terminal: “first VH, first VL, second VH, second VL”; “second VH, second VL, first VH, first VL”; “first VL, first VH, second VL, second VH”; “second VL, second VH, first VL, first VH”; “first VH, first VL, second VL, second VH”; “second VH, second VL, first VL, first VH”; “first VL, first VH, second VH, second VL”; “second VL, second VH, first VH, first VL”; “first VL, second VL, second VH, first VH”; “second VL, first VL, first VH, second VH”; “first VH, second VL, second VH, first VL”; “second VH, first VL, first VH, second VL”; “first VL, second VH, second VL, first VH”; “second VL, first VH, first VL, second VH”; “first VH, second VH, second VL, first VL”; or “second VH, first VH, first VL, second VL”. Any of the above-mentioned adjacent variable regions are independently connected by a linker, and the linker between the adjacent variable regions may be the same or different. In certain embodiments, the linker is a flexible linker. In certain embodiments, the linker is a polypeptide having a sequence as set forth in (GGGGS)x1 or (EAAAK)x2, wherein x1 and x2 are independently selected from integers from 1 to 6; in certain embodiments, the linker is a polypeptide containing the sequence as set forth in SEQ ID NO:68. In certain embodiments, the linker is selected from a polypeptide of the sequence as set forth in SEQ ID NO: 17, 18, 19, 20 or 68. The first VH has a sequence as set forth in SEQ ID NO: 1 or variant thereof; the first VL has a sequence as set forth in SEQ ID NO: 2 or variant thereof; the second VH has a sequence as set forth in SEQ ID NO: 3 or 76 or variant thereof; the second VL has a sequence as set forth in SEQ ID NO: 4 or 77 or variant thereof; wherein the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% compared to the sequence from which it is derived, or has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In certain embodiments, the antigen-binding domain comprises, from N-terminal to C-terminal:

(1) the first VL as set forth in SEQ ID NO: 2, the linker as set forth in SEQ ID NO: 18, the first VH as set forth in SEQ ID NO: 1, the linker as set forth in SEQ ID NO: 19, the second VH as set forth in SEQ ID NO: 3, the linker as set forth in SEQ ID NO: 18 and the second VL as set forth in SEQ ID NO: 4;

(2) the first VL as set forth in SEQ ID NO: 2, the linker as set forth in SEQ ID NO: 18, the first VH as set forth in SEQ ID NO: 1, the linker as set forth in SEQ ID NO: 20, the second VH as set forth in SEQ ID NO: 3, the linker as set forth in SEQ ID NO: 18 and the second VL as set forth in SEQ ID NO: 4;

(3) the second VH as set forth in SEQ ID NO: 3, the linker as set forth in SEQ ID NO: 18, the second VL as set forth in SEQ ID NO: 4, the linker as set forth in SEQ ID NO: 19, the first VL as set forth in SEQ ID NO: 2, the linker as set forth in SEQ ID NO: 18 and the first VH as set forth in SEQ ID NO: 1;

(4) the second VH as set forth in SEQ ID NO: 3, the linker as set forth in SEQ ID NO: 18, the second VL as set forth in SEQ ID NO: 4, the linker as set forth in SEQ ID NO: 20, the first VL as set forth in SEQ ID NO: 2, the linker as set forth in SEQ ID NO: 18 and the first VH as set forth in SEQ ID NO: 1;

(5) the first VL as set forth in SEQ ID NO: 2, the linker as set forth in SEQ ID NO: 17, the first VH as set forth in SEQ ID NO: 1, the linker as set forth in SEQ ID NO: 19, the second VH as set forth in SEQ ID NO: 3, the linker as set forth in SEQ ID NO: 17 and the second VL as set forth in SEQ ID NO: 4;

(6) the second VH as set forth in SEQ ID NO: 3, the linker as set forth in SEQ ID NO: 17, the first VL as set forth in SEQ ID NO: 2, the linker as set forth in SEQ ID NO: 19, the first VH as set forth in SEQ ID NO: 1, the linker as set forth in SEQ ID NO: 17 and the second VL as set forth in SEQ ID NO: 4;

(7) the first VL as set forth in SEQ ID NO: 2, the linker as set forth in SEQ ID NO: 18, the first VH as set forth in SEQ ID NO: 1, the linker as set forth in SEQ ID NO: 19, the second VH as set forth in SEQ ID NO: 3, the linker as set forth in SEQ ID NO:68 and the second VL as set forth in SEQ ID NO:4; or

(8) the first VL as set forth in SEQ ID NO: 2, the linker as set forth in SEQ ID NO: 18, the first VH as set forth in SEQ ID NO: 1, the linker as set forth in SEQ ID NO: 19, the second VH as set forth in SEQ ID NO: 76, the linker as set forth in SEQ ID NO:68 and the second VL as set forth in SEQ ID NO:77.

CAR Construct

In embodiments of the present invention, the present invention also provides a CAR construct, which comprises independent multiple (e.g., two, three, four or more) CARs, each CAR binds to a single antigen and exists individually on a cell surface, and has an antigen specificity for its respective target and can induce an antigen-specific response. For example, the nucleotide sequences encoding each CAR can be linked by sequences encoding self-cleaving peptides, so that each CAR is separately/simultaneously cleaved and released after the full sequence of the CAR construct is fully translated; or, one CAR can be cleaved before the next CAR is translated, thereby releasing each CAR (e.g., the first CAR and the second CAR). In embodiments, such CAR construct may comprise two independent CARs, for example, bicistronic CAR (BiCAR). Examples of such CAR herein include at least the BiCAR as described below.

In this aspect, the present invention also provides a CAR construct targeting BCMA and CD19, the CAR construct comprising independently a first CAR and a second CAR, wherein the first CAR comprises a first antibody or antigen-binding fragment thereof targeting BCMA, a spacer domain, a transmembrane domain, and an intracellular signaling domain; the second CAR comprises a second antibody or antigen-binding fragment thereof targeting CD19, a spacer domains, a transmembrane domain, and an intracellular signaling domain; wherein the first antibody or antigen-binding fragment thereof and the second antibody or antigen-binding fragment thereof are as defined in any one of the preceding embodiments.

In certain embodiments, the first antibody or antigen-binding fragment thereof targeting BCMA or the second antibody or antigen-binding fragment thereof targeting CD19 is an scFv. In certain embodiments, the BCMA-targeting scFv comprises a first VL as set forth in SEQ ID NO:2, a linker as set forth in SEQ ID NO:17, 18, 19, 20 or 68, a first VH as set forth in SEQ ID NO:1; the CD19-targeting scFv comprises a second VL as set forth in SEQ ID NO: 4 or 77, a linker as set forth in SEQ ID NO: 17, 18, 19, 20 or 68, a second VH as set forth in SEQ ID NO: 3 or 76. In certain embodiments, the BCMA-targeting scFv has a sequence as set forth in SEQ ID NO: 25 or 27, and the CD19-targeting scFv has a sequence as set forth in SEQ ID NO: 26 or 28.

Transmembrane Domain of CAR or CAR Construct

The transmembrane domain contained in the CAR or CAR construct of the present invention can be any protein structure known in the art as long as it can be thermodynamically stable in cell membranes (especially eukaryotic cell membranes). Transmembrane domains suitable for use in the CAR or CAR construct of the present invention may be derived from a natural source. In such embodiments, the transmembrane domain can be derived from any membrane-bound or transmembrane protein. Alternatively, the transmembrane domain may be a synthetic non-naturally occurring protein segment, for example, a protein segment comprising a predominantly hydrophobic residue such as leucine and valine.

In certain embodiments, the transmembrane domain is a transmembrane region of a protein selected from the group consisting of: α, β or ζ chain of T cell receptor, CD3ε, CD3ζ, CD4, CD5, CD8α, CD28, CD137, CD152, CD154 and PD1, as well as any combination thereof. In certain preferred embodiments, the transmembrane domain is a transmembrane region of a protein selected from the group consisting of: CD8α, CD28, CD4, PD1, CD152 and CD154. In certain preferred embodiments, the transmembrane domain comprises the transmembrane region of CD8α or CD28. In certain exemplary embodiments, the transmembrane domain comprises the amino acid sequence as set forth in SEQ ID NO: 22 or 72.

Spacer Domain of CAR or CAR Construct

The chimeric antigen receptor or CAR construct of the present invention comprises a spacer domain between the antigen-binding domain and the transmembrane domain.

In certain embodiments, the spacer domain comprises the CH2 and CH3 regions of an immunoglobulin (e.g., IgG1 or IgG4). In such embodiments, without being bound by a particular theory, it is believed that CH2 and CH3 extend the antigen-binding domain of the CAR or CAR construct from the cell membrane of the cell expressing the CAR or CAR construct, and thus the size and domain structure of a native TCR may be more precisely mimicked.

In certain embodiments, the spacer domain comprises a hinge domain. The hinge domain can be an amino acid fragment commonly found between two domains in a protein, which may allow the protein to have flexibility and allow one or two domains to move relative to each other. Thus, the hinge domain can be any amino acid sequence as long as it can provides the antigen-binding domain with such flexibility and such movement relative to the transmembrane domain.

In certain embodiments, the hinge domain is a hinge region or portion thereof of a naturally occurring protein. In certain embodiments, the hinge domain comprises the hinge region of CD8α or a portion thereof, for example, a fragment comprising at least 15 (e.g., 20, 25, 30, 35 or 40) contiguous amino acids of the hinge region of CD8α or IgG4.

In certain embodiments, the spacer domain comprises a hinge domain, and the hinge domain comprises the hinge region of PD1, CD152 or CD154. In certain embodiments, the spacer domain comprises a fragment of at least 15 (e.g., 20, 25, 30, 35, or 40) contiguous amino acids of the hinge region of PD1, CD152, or CD154. In certain exemplary embodiments, the spacer domain comprises the amino acid sequence as set forth in SEQ ID NO: 21 or 70.

Signal Peptide of CAR or CAR Construct

In certain embodiments, the CAR or CAR construct of the present invention may further comprise a signal peptide at its N-terminal. For example, the first CAR and the second CAR of the CAR construct may further comprise signal peptides at their N-terminal, respectively. Typically, a signal peptide is a polypeptide sequence that targets the sequence to which it is linked to a desired site in a cell. In certain embodiments, the signal peptide can target the CAR or CAR construct to which it is linked to the secretory pathway of the cell, and allow the CAR or CAR construct to be further integrated and anchored into the lipid bilayer. Signal peptides useful in CARs or CAR constructs are known to those of skill in the art. In certain embodiments, the signal peptide comprises a heavy chain signal peptide (e.g., heavy chain signal peptide of IgG1), a granulocyte-macrophage colony stimulating factor receptor 2 (GM-CSFR2) signal peptide, or a CD8α signal peptide. In certain preferred embodiments, the signal peptide is selected from CD8α signal peptides. In certain exemplary embodiments, the signal peptide comprises the amino acid sequence as set forth in SEQ ID NO:49.

Intracellular Signaling Domain of CAR or CAR Construct

The intracellular signaling domain contained in the CAR or CAR construct of the present invention is involved in the transduction of the signal of an effective antigen-receptor binding (binding of the CAR or CAR construct of the present invention to BCMA and CD19) into immune effector cells, activating at least one normal effector function of immune effector cells expressing the CAR or CAR construct, or enhancing the secretion of at least one cytokine (e.g., IL-2, IFN-γ, etc.) of immune effector cells expressing the CAR or CAR construct.

In certain embodiments, the intracellular signaling domain comprises a primary signaling domain and/or a costimulatory signaling domain.

In the present invention, the primary signaling domain may be any intracellular signaling domain comprising an immunoreceptor tyrosine activation motif (ITAM). In certain embodiments, the primary signaling domain comprises an immunoreceptor tyrosine activation motif (ITAM). In certain embodiments, the primary signaling domain comprises an intracellular signaling domain of a protein selected from the group consisting of: CD3ζ, FcRγ, FcRβ, CD3γ, CD3δ, CD3ε, CDS, CD22, CD79a, CD79b, and CD66d. In certain embodiments, the primary signaling domain comprises the intracellular signaling domain of CD3.

In the present invention, the costimulatory signaling domain may be an intracellular signaling domain from a costimulatory molecule. In certain embodiments, the costimulatory signaling domain comprises an intracellular signaling domain of a protein selected from the group consisting of: CARD11, CD2, CD7, CD27, CD28, CD30, CD134 (OX40), CD137 (4-1BB), CD150 (SLAMF1), CD270 (HVEM), and DAP10.

In certain embodiments, the costimulatory signaling domain is selected from the intracellular signaling domain of CD28, or the intracellular signaling domain of CD137(4-1BB), or a combination of fragments of both.

In certain embodiments, the intracellular signaling domain comprises one costimulatory signaling domain. In certain embodiments, the intracellular signaling domain comprises two or more costimulatory signaling domains. In such embodiments, the two or more costimulatory signaling domains may be the same or different.

In certain embodiments, the intracellular signaling domain comprises a primary signaling domain and at least one costimulatory signaling domain. The primary signaling domain and at least one costimulatory signaling domain can be linked in series to the carboxy-terminal of the transmembrane domain in any order.

In certain embodiments, the intracellular signaling domain may comprise the intracellular signaling domain of CD3ζ and the intracellular signaling domain of CD137. In certain exemplary embodiments, the intracellular signaling domain of CD3ζ comprises the amino acid sequence as set forth in SEQ ID NO: 24 or 74. In certain exemplary embodiments, the intracellular signaling domain of CD137 comprises the amino acid sequence as set forth in SEQ ID NO:23.

Full-Length CAR or CAR Construct

The CAR provided by the present invention comprises an antigen-binding domain, a spacer domain, a transmembrane domain, and an intracellular signaling domain in sequence from its N-terminal to its C-terminal. In certain preferred embodiments, the intracellular signaling domain comprises a costimulatory signaling domain and a primary signaling domain from the N-terminal to the C-terminal.

In certain embodiments, the spacer domain comprises the hinge region of CD8 (e.g., CD8α) or IgG4, which has a sequence as set forth in SEQ ID NO: 21 or 70. In certain embodiments, the transmembrane domain comprises the transmembrane region of CD8 (e.g., CD8α) or CD28, which has a sequence as set forth in SEQ ID NO: 22 or 72.

In certain embodiments, the intracellular signaling domain comprises a primary signaling domain and a costimulatory signaling domain, wherein the primary signaling domain comprises the intracellular signaling domain of CD3ζ, which has a sequence as set forth in SEQ ID NO: 24 or 74. The costimulatory signaling domain comprises the intracellular signaling domain of CD137, which has a sequence as set forth in SEQ ID NO:23.

In certain preferred embodiments, the chimeric antigen receptor comprises the signal peptide, the antigen-binding domain, the spacer domain, the transmembrane domain, the intracellular signaling domain (with the co-stimulatory signaling domain and the primary signaling domain from its N-terminal to C-terminal) in sequence from its N-terminal to its C-terminal.

In certain exemplary embodiments, the signal peptide comprises an IgG1 heavy chain signal peptide or a CD8α signal peptide. In certain exemplary embodiments, the signal peptide comprises a CD8α signal peptide having the sequence as set forth in SEQ ID NO:49.

In certain exemplary embodiments, the antigen-binding domain of the chimeric antigen receptor CAR of the present invention comprises a first antigen-binding domain (specifically binding BCMA) and a second antigen-binding domain (specifically binding CD19), the first antigen-binding domain comprises a first VH and a first VL, the second antigen-binding domain comprises a second VH and a second VL, wherein the first VH, the first VL, the second VH and second VL can be positioned relative to each other from N-terminal to C-terminal in any suitable arrangement, for example, VH_{(first/second)}-VL_{(first/second)}-VH_{(first/second)}-VL_{(first/second)}, VH_{(first/second)}-VL_{(first/second)}-VL_{(first/second)}-VH_{(first/second)}, VL_{(first/second)}-VH_{(first/second)}-VL_{(first/second)}-VH_{(first/second)} or VL_{(first/second)}-VH_{(first/second)}-VH_{(first/second)}-VL_{(first/second)}, VH_{(first/second)}-VH_{(first/second)}-VL_{(first/second)}-VL_{(first/second)} or VL_{(first/second)}-VL_{(first/second)}-VH_{(first/second)}-VH_{(first/second)}; in which “first/second” in brackets means that either “first antigen-binding domain” or “second antigen-binding domain” is selected; more preferably, the VH regions and the VL regions of the first and second antigen-binding domains comprise the following in sequence from N-terminal to C-terminal: “first VH, first VL, second VH, second VL”; “second VH, second VL, first VH, first VL”; “first VL, first VH, second VL, second VH”; “second VL, second VH, first VL, first VH”; “first VH, first VL, second VL, second VH”; “second VH, second VL, first VL, first VH”; “first VL, first VH, second VH, second VL”; “second VL, second VH, first VH, first VL”; “first VL, second VL, second VH, first VH”; “second VL, first VL, first VH, second VH”; “first VH, second VL, second VH, first VL”; “second VH, first VL, first VH, second VL”; “first VL, second VH, second VL, first VH”; “second VL, first VH, first VL, second VH”; “first VH, second VH, second VL, first VL”; or “second VH, first VH, first VL, second VL”. Any of the above-mentioned adjacent variable regions are independently connected by a linker, and the linker between the adjacent variable regions may be the same or different. In certain embodiments, the linker is a flexible linker. In certain embodiments, the linker is a polypeptide having a sequence as set forth in (GGGGS)x1 or (EAAAK)x2, wherein x1 and x2 are independently selected from integers from 1 to 6; in certain embodiments, the linker is a polypeptide containing the sequence as set forth in SEQ ID NO:68. In certain embodiments, the linker is selected from a polypeptide of the sequence as set forth in SEQ ID NO: 17, 18, 19, 20 or 68.

In certain embodiments, the TanCAR has an amino acid sequence selected from the group consisting of: (1) an amino acid sequence as set forth in any one of SEQ ID NOs: 37-42, 64, 66; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in any one of SEQ ID NO: 37-42, 64, 66, and the sequence substantially retaining at least one biological activity of the amino acid sequence from which it is derived (e.g., ability to direct the specificity and reactivity of an immune effector cell to a cell expressing BCMA and CD19 in a non-MHC-restricted manner).

The CAR construct provided by the present invention comprises independently a first CAR and a second CAR, wherein the first CAR comprises a first antibody or antigen-binding fragment thereof targeting BCMA, a spacer domain, a transmembrane domain and an intracellular signaling domain from its N-terminal to its C-terminal; the second CAR comprises a second antibody or antigen-binding fragment thereof targeting CD19, a spacer domain, a transmembrane domain, and an intracellular signaling domain from its N-terminal to its C-terminal. In certain preferred embodiments, the first CAR and/or the second CAR comprises a signal peptide, an antigen-binding domain, a spacer domain, a transmembrane domain, an intracellular signaling domain (with a costimulatory signaling domain and a primary signaling domain from N-terminal to C-terminal) from its N-terminal to its C-terminal in sequence.

In certain embodiments, the first CAR has an amino acid sequence selected from the group consisting of: (1) an amino acid sequence as set forth in SEQ ID NO:29; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% compared to the amino acid sequence as set forth in SEQ ID NO:29. In certain embodiments, the second CAR has an amino acid sequence selected from the group consisting of: (1) an amino acid sequence as set forth in SEQ ID NO:30; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% compared to the amino acid sequence as set forth in SEQ ID NO: 30.

In certain exemplary embodiments, the CAR construct has an amino acid sequence selected from the group consisting of: (1) an amino acid sequence as set forth in SEQ ID NO:51, (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 51, and the sequence substantially retaining at least one biological activity of the amino acid sequence from which it is derived (e.g., ability to direct the specificity and reactivity of an immune effector cell to a cell expressing BCMA and CD19 in a non-MHC-restricted manner). The amino acid sequence of the CAR construct described herein refers to an amino acid sequence corresponding to the nucleotide sequence of the nucleic acid molecule encoding the CAR construct.

Preparation of Chimeric Antigen Receptor/CAR Construct

Methods for generating a chimeric antigen receptor and an immune effector cell (e.g., T cell) comprising the chimeric antigen receptor are known in the art, and may comprise transfecting a cell with at least one polynucleotide encoding a CAR or CAR construct, and expressing the polynucleotide in the cell. For example, a nucleic acid molecule encoding the CAR or CAR construct of the present invention can be contained in an expression vector (e.g., a lentiviral vector), the expression vector can be expressed in a host cell, such as a T cell, to prepare the CAR or CAR constructs (e.g., TanCAR or BiCAR).

Accordingly, in yet another aspect, the present invention provides an isolated nucleic acid molecule, which comprises a nucleotide sequence encoding the chimeric antigen receptor CAR of the present invention. In certain exemplary embodiments, the nucleotide sequence encoding the chimeric antigen receptor of the present invention is selected from the group consisting of: (1) a nucleotide sequence as set forth in any one of SEQ ID NOs: 43-48, 65, 67; (2) a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% compared to the nucleotide sequence as set forth in any one of SEQ ID NOs: 43-48, 65, 67, and the sequence substantially retaining at least one biological activity of the nucleotide sequence from which it is derived (e.g., ability to encode a sequence capable of directing the specificity and reactivity of an immune effector cell to a cell expressing BCMA and CD19 in a non-MHC-restricted manner).

The present invention also provides a nucleic acid construct, which comprising a first nucleotide sequence encoding a first CAR in the CAR construct of the present invention, and a second nucleotide sequence encoding a second CAR in the CAR construct of the present invention. In certain embodiments, the first nucleotide sequence and the second nucleotide sequence are linked in any order by a nucleotide sequence encoding a self-cleaving peptide (e.g., P2A, E2A, F2A or T2A). In certain embodiments, the self-cleaving peptide is P2A (e.g., P2A of the sequence as set forth in SEQ ID NO:50).

In certain exemplary embodiments, the nucleic acid construct comprises a nucleotide sequence selected from the group consisting of: (1) a nucleotide sequence as set forth in SEQ ID NO: 52; (2) a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% compared to the nucleotide sequence as set forth in SEQ ID NO: 52, and the sequence substantially retaining at least one biological activity of the nucleotide sequence from which it is derived (e.g., ability to encode a sequence capable of directing the specificity and reactivity of an immune effector cell to a cell expressing BCMA and CD19 in a non-MHC-restricted manner).

Those skilled in the art understand that, due to the degeneracy of the genetic code, a nucleotide sequence encoding the chimeric antigen receptor CAR or CAR construct of the present invention may have a variety of different sequences. Thus, unless stated otherwise, “nucleotide sequence encoding an amino acid sequence” includes all nucleotide sequences that are a degenerate form of each other and encode the same amino acid sequence.

In another aspect, the present invention provides a vector (e.g., a cloning vector or an expression vector), which comprises the isolated nucleic acid molecule or nucleic acid construct as described above.

In certain embodiments, the vector comprises a nucleotide sequence encoding the chimeric antigen receptor CAR or CAR construct of the present invention.

In certain exemplary embodiments, the nucleotide sequence encoding the chimeric antigen receptor of the present invention is selected from the group consisting of: (1) a nucleotide sequence as set forth in any one of SEQ ID NOs: 43-48, 65, 67; (2) a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% compared to the nucleotide sequence as set forth in any one of SEQ ID NOs: 43-48, 65, 67, and the sequence substantially retaining at least one biological activity of the nucleotide sequence from which it is derived (e.g., ability to encode a sequence capable of directing the specificity and reactivity of an immune effector cell to a cell expressing BCMA and CD19 in a non-MHC-restricted manner).

In certain exemplary embodiments, the nucleotide sequence encoding the CAR construct comprises a nucleotide sequence selected from the group consisting of: (1) a nucleotide sequence as set forth in SEQ ID NO: 52; (2) a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% compared to the nucleotide sequence as set forth in SEQ ID NO: 52, and the sequence substantially retaining at least one biological activity of the nucleotide sequence from which it is derived (e.g., ability to encode a sequence capable of directing the specificity and reactivity of an immune effector cell to a cell expressing BCMA and CD19 in a non-MHC-restricted manner).

In certain embodiments, the vector is selected from the group consisting of DNA vector, RNA vector, plasmid, transposon vector, CRISPR/Cas9 vector, viral vector.

In certain embodiments, the vector is an expression vector.

In certain embodiments, the vector is an episomal vector.

In certain embodiments, the vector is a viral vector.

In certain exemplary embodiments, the viral vector is a lentiviral vector, adenoviral vector, or retroviral vector.

In certain embodiments, the vector is an episomal or non-integrating viral vector, such as an integration-deficient retrovirus or lentivirus.

In yet another aspect, the present invention provides a host cell, which comprises the isolated nucleic acid molecule, nucleic acid construct or vector as described above. The vector described above can be introduced into a host cell by various suitable means, such as calcium phosphate transfection, DEAE-dextran-mediated transfection, microinjection, electroporation, TALEN method, ZFN method, non-viral vector-mediated transfection (e.g., liposome) or viral vector-mediated transfection (e.g., lentiviral infection, retroviral infection, adenoviral infection), and other physical, chemical or biological methods for transfer into host cells, such as transposon technology, CRISPR-Cas9 and other technologies.

In certain embodiments, the host cell expresses the chimeric antigen receptor CAR or CAR construct of the present invention.

In certain embodiments, the host cell is selected from mammalian (e.g., human) immune cells. In certain embodiments, the immune cell is derived from a patient or healthy donor. In certain embodiments, the immune cell is selected from T lymphocyte, natural killer (NK) cell, monocyte, macrophage or dendritic cell, and any combination thereof.

In certain embodiments, the host cell or immune cell comprises the isolated nucleic acid molecule, nucleic acid construct or vector of the present invention.

In another aspect, the present invention provides a method for preparing a cell expressing any of the aforementioned chimeric antigen receptor or CAR construct of the present invention, comprising: (1) providing a host cell; (2) obtaining a host cell capable of expressing the chimeric antigen receptor or CAR construct of the present invention; wherein step (2) comprises: introducing the isolated nucleic acid molecule, nucleic acid construct or vector of the present invention into the host cell. The isolated nucleic acid molecule or a vector comprising the same comprises a nucleotide sequence encoding the chimeric antigen receptor of the present invention. Alternatively, the nucleic acid construct or a vector comprising the same comprises a nucleotide sequence encoding the CAR construct of the present invention.

In certain embodiments, the host cell is selected from immune cells, such as T lymphocyte, NK cell, monocyte, dendritic cell, macrophage, and any combination thereof. In certain embodiments, the immune cell is selected from T lymphocyte, NK cell, monocyte, macrophage, or dendritic cell, and any combination thereof.

In certain embodiments, in step (1), the immune cell undergoes pretreatment; the pretreatment comprises sorting, activation, and/or proliferation of the immune cell; in certain embodiments, the pretreatment comprises contacting the immune cell with an anti-CD3 antibody and an anti-CD28 antibody, thereby stimulating the immune cell and inducing its proliferation, thereby generating a pretreated immune cell.

In certain embodiments, in step (2), the nucleic acid molecule or vector is introduced into the host cell by viral infection. In certain embodiments, in step (2), the nucleic acid molecule or vector is introduced into the host cell by means of non-viral vector transfection, for example by a method such as transposon-based vector system, CRISPR/Cas9 vector, TALEN method, ZFN method, electroporation method, calcium phosphate transfection, DEAE-dextran-mediated transfection or microinjection.

In certain embodiments, after step (2), the method further comprises: expanding the host cell obtained in step (2).

Engineered Immune Cell

An immune cell derived from a patient or healthy donor can be engineered as an immune cell expressing CAR or CAR construct targeting BCMA and CD19 by the preparation method provided in the present invention.

Therefore, in another aspect, the present invention also provides an engineered immune cell. The engineered immune cell expresses the CAR or CAR construct targeting BCMA and CD19 of the present invention (e.g., TanCAR or BiCAR). The CAR targeting BCMA and CD19 is expressed on the surface of the engineered immune cell; the CAR construct targeting BCMA and CD19 is expressed on the surface of the engineered immune cell in the form of co-expression of the chimeric antigen receptor targeting BCMA and chimeric antigen receptor targeting CD19. In certain embodiments, the immune cell is derived from T lymphocyte, NK cell, monocyte, macrophage, or dendritic cell, and any combination thereof of a patient or healthy donor. The immune cell is obtained from a patient or healthy donor. The immune cell is introduced with the isolated nucleic acid molecule, nucleic acid construct or vector of the present invention by the method described herein, thereby preparing an engineered immune cell. The engineered immune cell therefore expresses the CAR or CAR construct targeting BCMA and CD19 of the present invention.

In certain embodiments, the engineered immune cell also expresses a CAR that is not specific for BCMA/CD19, such as a CAR that is specific for CD20, CD22, CD33, CD123, or CD138.

In certain embodiments, the engineered immune cell further comprises knockout of one or more endogenous genes, wherein the endogenous genes comprise genes encoding TCRα, TCRβ, CD52, glucocorticoid receptor (GR), deoxycytidine kinase (dCK), or immune checkpoint protein, such as programmed death-1 (PD-1).

Immune Cell Composition

In another aspect, the present invention also provides an immune cell composition, the immune cell composition comprises the aforementioned engineered immune cell, and optionally an unengineered and/or unsuccessfully engineered immune cell, and the unengineered and/or unsuccessfully engineered immune cell does not express a CAR specific for BCMA and CD19. Limited to the current state of technology and for unknown reasons, not all immune cells can be engineered to express a CAR or CAR construct specific for BCMA and CD19. Moreover, an immune cell that does not express CAR may also have a certain biological activity, so the immune cell composition may contain an immune cell that expresses CAR specific for BCMA and CD19 and an immune cell that does not express CAR specific for BCMA and CD19, and the immune cell composition can still meet the needs of clinical applications. In certain embodiments, the engineered immune cells expressing a chimeric antigen receptor CAR or CAR construct specific for BCMA and CD19 account for about 10% to 100%, preferably 40% to 80%, of the total cells of the immune cell composition.

In some embodiments, the immune cell composition is cultured to form an immune cell line, therefore, in another aspect, the present invention also provides an immune cell line comprising the immune cell composition.

In another aspect, the present invention provides a kit for preparing a chimeric antigen receptor CAR or CAR construct targeting BCMA and CD19, or for preparing a cell expressing the chimeric antigen receptor CAR or CAR construct. In certain embodiments, the kit comprises the isolated nucleic acid molecule or nucleic acid construct of the present invention, or a vector comprising the above-described nucleic acid molecule or nucleic acid construct, or a host cell comprising the above-described nucleic acid molecule or nucleic acid construct or vector, and necessary solvent, such as sterile water, physiological saline, or cell culture medium, such as LB medium, such as EliteCell primary T lymphocyte culture system (product number: PriMed-EliteCell-024), and optionally, also comprises an instruction manual.

In another aspect, the present invention provides use of the aforementioned kit for preparing a chimeric antigen receptor CAR or CAR construct targeting BCMA and CD19 or a cell expressing the chimeric antigen receptor CAR or CAR construct.

The present invention also provides an intermediate product in the process of preparing the chimeric antigen receptor CAR or CAR construct or a host cell comprising the chimeric antigen receptor CAR or CAR construct, for example, a nucleic acid encoding an antibody or antigen-binding fragment targeting BCMA and/or CD19, a vector containing a nucleic acid encoding the antibody or antigen-binding fragment or the chimeric antigen receptor CAR or CAR construct, or bacteria, yeast, virus, or viral particle capable of infecting a host cell, such as free lentiviral particle, herpes simplex virus, that comprises a sequence of the CAR or CAR construct targeting BCMA and CD19 of the invention.

Pharmaceutical Composition

In another aspect, the present invention provides a pharmaceutical composition, which comprises the antibody or antigen-binding fragment thereof, chimeric antigen receptor CAR or CAR construct, isolated nucleic acid molecule, nucleic acid construct, vector, host cell, engineered immune cell or immune cell composition of the present invention, and a pharmaceutically acceptable carrier and/or excipient.

In certain embodiments, the pharmaceutical composition may further comprise an additional pharmaceutically active agent.

In certain embodiments, the additional pharmaceutically active agent includes, but is not limited to, an additional antibody, fusion protein, or drug (e.g., antineoplastic drug, such as drug used in radiation therapy or chemotherapeutic drug). In certain embodiments, the additional pharmaceutically active agent has antitumor activity.

In certain embodiments, in the pharmaceutical composition, the chimeric antigen receptor CAR or CAR construct of the present invention, isolated nucleic acid molecule, nucleic acid construct, vector, host cell, engineered immune cell or immune cell composition, and the additional pharmaceutically active agent may be provided as separate components or as mixed components. Thus, the chimeric antigen receptor CAR or CAR construct, isolated nucleic acid molecule, nucleic acid construct, vector, host cell, engineered immune cell or immune cell composition of the present invention and the additional pharmaceutically active agent can be administered simultaneously, separately or sequentially.

In certain embodiments, the pharmaceutical composition of the present invention comprises: the antibody or antigen-binding fragment thereof of the present invention, the chimeric antigen receptor CAR or CAR construct of the present invention, the isolated nucleic acid molecule, nucleic acid construct, vector, host cell of the present invention, the engineered immune cell or immune cell composition of the present invention.

In certain embodiments, the pharmaceutical composition of the present invention comprises: the engineered immune cell or immune cell composition of the present invention.

The isolated nucleic acid molecule, nucleic acid construct, vector, host cell, engineered immune cell or immune cell composition of the present invention can be formulated into any dosage form known in the medical art, for example, tablet, pill, suspension, emulsion, solution, gel, capsule, powder, granule, elixir, lozenge, suppository, injection (including injection, sterile powder for injection, and concentrated solution for injection), inhalant, spray, etc. The preferred dosage form depends on the intended mode of administration and therapeutic use. The pharmaceutical composition of the present invention should be sterile and stable under the conditions of manufacture and storage. A preferred dosage form is an injection. Such injection can be a sterile injection solution. In addition, the sterile injection solution can be prepared as a sterile lyophilized powder (e.g., by vacuum drying or freeze-drying) for ease of storage and use. Such sterile lyophilized powder can be dispersed in a suitable vehicle such as water for injection (WFI), bacteriostatic water for injection (BWFI), sodium chloride solution (e.g., 0.9% (w/v) NaCl), glucose solution (e.g., 5% glucose), surfactant-containing solution (e.g., 0.01% polysorbate-20), pH buffer solution (e.g., phosphate buffered solution), Ringer's solution, and any combination thereof.

The isolated nucleic acid molecule, nucleic acid construct, vector, host cell, engineered immune cell or immune cell composition of the present invention can be administered by any suitable method known in the art, including, but not limited to, oral, buccal, sublingual, ocular, topical, parenteral, rectal, intrathecal, intracytoplasmic reticulum, inguinal, intravesical, topical (e.g., powder, ointment, or drop), or nasal administration route. However, for many therapeutic uses, the preferred route/mode of administration is parenteral administration (e.g., intravenous or bolus injection, subcutaneous injection, intraperitoneal injection, intramuscular injection). It will be understood by those skilled in the art that the route and/or mode of administration will vary depending on the intended purpose. In certain embodiments, the isolated nucleic acid molecule, nucleic acid construct, vector, host cell, engineered immune cell or immune cell composition of the present invention is administered by intravenous injection or bolus injection.

The pharmaceutical composition of the present invention may comprise a “therapeutically effective amount” or “prophylactically effective amount” of the isolated nucleic acid molecule, nucleic acid construct, vector, host cell, engineered immune cell or immune cell composition of the present invention. The “prophylactically effective amount” refers to an amount sufficient to prevent, stop or delay the development of a disease. The “therapeutically effective amount” refers to an amount sufficient to cure or at least partially arrest a disease and its complication in a patient already suffering from the disease. The therapeutically effective amount of the isolated nucleic acid molecule, nucleic acid construct, vector, host cell, engineered immune cell or immune cell composition of the present invention may vary depending on factors such as the severity of the disease to be treated, the patient's own general state of immune system, the patient's general conditions such as age, weight and sex, mode of administration, and additional therapy applied simultaneously, etc.

In the present invention, the dosing regimen can be adjusted to obtain the optimal response of interest (e.g., a therapeutic or prophylactic response). For example, it can be administered in a single dose, can be administered multiple times over a period of time, or the dose can be reduced or increased proportionally according to the urgency of the treatment situation.

Therapeutic Method and Use

In another aspect, the present invention provides a method for preventing and/or treating a B cell-related disease or condition in a subject (e.g., a human), the method comprising administering to a subject in need thereof an effective amount of the isolated nucleic acid molecule, nucleic acid construct, vector, host cell, engineered immune cell or immune cell composition or pharmaceutical composition of the present invention.

In certain embodiments, the method comprises administering to the subject an effective amount of the isolated nucleic acid molecule, nucleic acid construct, vector, host cell, engineered immune cell or immune cell composition of the present invention. In certain embodiments, the host cell is an immune cell (e.g., human immune cell).

In certain embodiments, the method for preventing and/or treating a B cell-related disease or condition in a subject (e.g., a human) comprises the following steps: (1) providing an immune cell required by the subject (e.g., T lymphocyte, NK cell, monocyte, macrophage, dendritic cell, or any combination thereof); (2) introducing a polynucleotide encoding the chimeric antigen receptor CAR or CAR construct of the present invention into the immune cell described in step (1) to obtain an immune cell expressing the chimeric antigen receptor CAR or CAR construct; (3) administering the immune cell obtained in step (2) to the subject for treatment.

In certain embodiments, the method comprises administering to the subject the immune cell expressing the CAR or CAR construct of the present invention in divided doses, for example, in one, two, three or more divided doses; for example, administering a first percentage of the total dose on the first treatment day, and administering a second percentage of the total dose on a subsequent (e.g., second, third, fourth, fifth, sixth, or seventh or later) treatment day; for example, administering a third percentage of the total dose (e.g., the remaining percentage) on a subsequent (e.g., third, fourth, fifth, sixth, seventh, eighth, ninth, tenth, or later) treatment day.

In certain embodiments, 10% of the total dose of cells is administered on the first treatment day, 30% of the total dose of cells is administered on the second day, and the remaining 60% of the total dose of cells is administered on the third day.

In certain embodiments, 50% of the total dose of cells is administered on the first treatment day, and 50% of the total dose of cells is administered on a subsequent (e.g., second, third, fourth, fifth, sixth, or seventh or later) treatment day. In certain embodiments, 1/3 of the total dose of cells is administered on the first treatment day, 1/3 of the total dose of cells is administered on a subsequent (e.g., second, third, fourth, fifth, sixth, or seventh or later) treatment day, and 1/3 of the total dose is administered on a subsequent (e.g., third, fourth, fifth, sixth, seventh, eighth, ninth, tenth, or later) treatment day.

In certain embodiments, the total dose of cells comprises 1 to 5×10⁷ or 1 to 5×10⁸ cells.

In certain embodiments, the physician may adjust the dosage or treatment regimen based on the patient's state, the size and stage of tumor, or clinical circumstances such as the drugs being used in combination therapy.

In certain embodiments, the B cell-related disease or condition is selected from the group consisting of multiple myeloma, non-Hodgkin's lymphoma, B cell proliferation of uncertain malignant potential, lymphomatoid granulomatosis, post-transplant lymphoproliferative disorder, immunomodulatory disorder, rheumatic arthritis, myasthenia gravis, idiopathic thrombocytopenic purpura, antiphospholipid syndrome, Chagas' disease, Graves' disease, Wegener's granulomatosis, polyarteritis nodosa, Sjogren's syndrome, pemphigus vulgaris, scleroderma, multiple sclerosis, antiphospholipid syndrome, ANCA-associated vasculitis, Goodpasture's disease, Kawasaki disease, autoimmune hemolytic anemia and rapidly progressive glomerulonephritis, heavy chain disease, primary or immunocyte-associated amyloidosis or monoclonal gammopathy of undetermined significance, systemic lupus erythematosus.

In certain embodiments, the B cell-related disease or condition is B cell- and plasma cell-related malignancy, B cell- and plasma cell-related autoimmune disease, such as multiple myeloma (MM) or non-Hodgkin's lymphoma (NHL). In certain embodiments, the B cell-related disease or condition is plasma cell malignancy.

In certain embodiments, the B cell-related disease or condition is an autoimmune disease associated with B cell and plasma cell, such as systemic lupus erythematosus.

In certain embodiments, the isolated nucleic acid molecule, nucleic acid construct, vector, host cell, engineered immune cell or immune cell composition of the present invention is administered in combination with an additional agent. In certain embodiments, the additional agent comprises: (i) an agent capable of increasing a function of a cell comprising the CAR or CAR construct nucleic acid or the CAR or CAR construct polypeptide (e.g., an immune cell expressing the CAR or CAR construct of the present invention, the engineered immune cell or immune cell composition of the present invention); (ii) an agent capable of ameliorating one or more side-effects associated with the administration of a cell comprising the CAR or CAR construct nucleic acid or the CAR or CAR construct polypeptide (e.g., an immune cell expressing the CAR or CAR construct of the present invention, the engineered immune cell or immune cell composition of the present invention); (iii) an additional agent for the treatment of a disease associated with BCMA and/or CD19. These agents can be administered before, concurrently with, or after the administration of the isolated nucleic acid molecule, vector, host cell, engineered immune cell or immune cell composition of the present invention.

In certain embodiments, the isolated nucleic acid molecule, nucleic acid construct, vector, host cell, engineered immune cell or immune cell composition of the present invention is administered in combination with an additional therapy. This additional therapy can be any therapy known for tumors, such as surgery, chemotherapy, radiation therapy, targeted therapy, immunotherapy, hormone therapy, gene therapy, or palliative care. Such additional therapy can be administered before, concurrently with, or after the administration of the isolated nucleic acid molecule, nucleic acid construct, vector, host cell, engineered immune cell or immune cell composition of the present invention.

In certain embodiments, the subject may be a mammal, such as a human.

In another aspect, there is provided use of the isolated nucleic acid molecule, nucleic acid construct, vector, host cell, engineered immune cell or immune cell composition of the present invention in the manufacture of a medicament, in which the medicament is used for preventing and/or treating a B cell-related disease or condition in a subject (e.g., a human). The dosage, dosage form, administration route, indication, combination therapy and other aspects of the aforementioned treatment methods can be applied to the use of the medicament.

Kit

In another aspect, the present invention provides a kit, which comprises the antibody or antigen-binding fragment thereof, chimeric antigen receptor CAR or CAR construct, nucleic acid molecule, nucleic acid construct, vector or host cell of the present invention.

In certain embodiments, the kit is used to prepare a chimeric antigen receptor CAR or CAR construct targeting BCMA and CD19, or to prepare a cell expressing the chimeric antigen receptor CAR or CAR construct.

### Definition of Terms

In the present invention, unless otherwise specified, scientific and technical terms used herein have the meanings commonly understood by those skilled in the art. In addition, the procedures of molecular biology, microbiology, cell biology, biochemistry, immunology and the like used herein are all routine steps widely used in the corresponding fields. Meanwhile, for a better understanding of the present invention, definitions and explanations of related terms are provided below.

As used herein, the term “BCMA” refers to a B cell maturation antigen. BCMA (also known as TNFRF17, BCM or CD269) is a member of the tumor necrosis factor receptor (TNFR) family and is predominantly expressed on terminally differentiated B cells, such as memory B cells and plasma cells. Its ligand is called B cell-activating factor (BAFF) and proliferation-inducing ligand (APRIL) of the TNF family. BCMA is involved in mediating plasma cell survival to maintain long-term humoral immunity. The gene for BCMA is encoded on chromosome 16 and produces a primary mRNA transcript of 994 nucleotides in length (NCBI accession number NM_001192.2), which encodes a protein of 184 amino acids (NP_001183.2). A second antisense transcript from the BCMA locus has been described that may play a role in regulating BCMA expression (Laabi et al. Y. et al, Nucleic Acids Res., 1994, 22:1147-1154). Additional transcript variants of unknown importance have been described (Smirnova A S et al. Mol Immunol., 2008, 45(4):1179-1183). A second isoform (also known as TV4) has been identified (Uniprot identifier Q02223-2). As used herein, “BCMA” comprises a protein comprising a mutation, such as comprising point mutation, fragment, insertion, deletion compared to a full-length wild-type BCMA and splice variant thereof.

As used herein, the term “CD19” refers to a B lymphocyte antigen CD19, also known as B lymphocyte surface antigen B4 or T cell surface antigen Leu-12, and includes any native CD19 of any vertebrate origin, including mammals, such as primate (e.g., human), non-human primate (e.g., cynomolgus monkey), and rodent (e.g., mouse and rat), unless otherwise specified. The amino acid sequence of human CD19 is at NCBI Accession No. NP_001171569. The term encompasses “full-length”, unprocessed human CD19 as well as any form of human CD19 derived from processing in cells as long as to which the antibody reported herein binds. CD19 is a structurally unique cell surface receptor expressed on the surface of human B cells, the B cells include but are not limited to pre-B cells, B cells in early development (i.e., immature B cells), mature B cells that differentiate terminally into plasma cells, and malignant B cells. CD19 is expressed by most of pre-B cell acute lymphoblastic leukemia (ALL), non-Hodgkin's lymphoma, B-cell chronic lymphocytic leukemia (CLL), prolymphocytic leukemia, hairy cell leukemia, common acute lymphocytic leukemia and some Null-acute lymphoblastic leukemias. The expression of CD19 on plasma cells further suggests that it may be expressed on tumors of differentiated B cell such as multiple myeloma. Therefore, an CD19 antigen is a target for immunotherapy for the treatment of non-Hodgkin's lymphoma, chronic lymphocytic leukemia and/or acute lymphoblastic leukemia.

As used herein, the term “antibody” refers to an immunoglobulin molecule capable of targeting a target (e.g., carbohydrate, polynucleotide, lipid, polypeptide, etc.) of BCMA and CD19 through at least one antigen recognition site located in the variable region of the immunoglobulin molecule. As used herein, the term comprises not only intact polyclonal or monoclonal antibody, but also fragment thereof (e.g., Fab, Fab′, F(ab′)₂, Fv), single chain antibody (e.g., scFv, di-scFv, (scFv)₂) and domain antibody (including, for example, shark and camel antibodies), as well as fusion protein including antibody, and immunoglobulin molecule in any other modified form comprising antigen recognition site. The antibody of the present invention is not limited by any particular method for producing the antibody. The antibody includes any type of antibody, such as IgG, IgA, or IgM (or a subclass thereof), and the antibody needs not be of any particular type. Depending on the amino acid sequence of the antibody heavy chain constant region, immunoglobulins can be assigned to different types. There are five main types of immunoglobulins: IgA, IgD, IgE, IgG, and IgM, several of which can be further divided into subclasses (isotypes), such as IgG1, IgG2, IgG3, IgG4, IgA1, and IgA2. The heavy chain constant regions corresponding to the different types of immunoglobulins are called α, δ, ε, γ, and μ, respectively. The antibody light chains can be classified as κ (kappa) and λ (lambda) light chains. The subunit structures and three-dimensional configurations of different types of immunoglobulins are well known. The heavy chain constant region consists of 4 domains (CH1, hinge region, CH2 and CH3). Each light chain consists of a light chain variable region (VL) and a light chain constant region (CL). The light chain constant region consists of one domain, CL. The constant domains are not directly involved in the binding of antibody to antigen, but exhibit a variety of effector functions, such as mediating the binding of immunoglobulin to a host tissue or factor, including various cells of immune system (e.g., effector cells) and the first component (C1q) of classical complement system.

The VH and VL regions of antibody can also be subdivided into regions of high variability (called as complementarity determining regions (CDRs)), interspersed with more conserved regions called as framework regions (FRs). Each V_{H} and V_{L} consists of 3 CDRs and 4 FRs arranged from amino-terminal to carboxy-terminal in the following order: FR1, CDR1, FR2, CDR2, FR3, CDR3, FR4. The variable regions (VH and VL) of each heavy chain/light chain pair respectively form the antigen binding site. The assignment of amino acids to regions or domains can follow the definitions of Kabat, Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883.

As used herein, the term “complementarity determining region” or “CDR” refers to the amino acid residues in the variable region of an antibody that are responsible for antigen binding. The variable regions of the heavy chain and light chain each contain three CDRs, designated CDR1, CDR2 and CDR3. The precise boundaries of these CDRs can be defined according to various numbering systems known in the art, for example according to the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883) or the IMGT numbering system (Lefranc et al., Dev. Comparat. Immunol. 27:55-77, 2003). For a given antibody, those skilled in the art will readily identify the CDRs defined by each numbering system. Also, correspondence between different numbering systems is well known to those skilled in the art (see, for example, Lefranc et al., Dev. Comparat. Immunol. 27:55-77, 2003).

In the present invention, the CDRs contained in the antibody or antigen-binding fragment thereof can be determined according to various numbering systems known in the art. In certain embodiments, the CDRs contained in the antibody or antigen-binding fragment thereof of the present invention are preferably determined by the Kabat, Chothia or IMGT numbering system. In certain embodiments, the CDRs contained in the antibody or antigen-binding fragment thereof of the present invention are identified by the Chothia numbering system. It is understood that in some embodiments, the CDRs may be a combination of Kabat and Chothia CDRs (also referred to as “combined CDRs” or “extended CDRs”). In some embodiments, the CDRs are Kabat CDRs. In other embodiments, the CDRs are Chothia CDRs. In other words, in embodiments with more than one CDR, the CDRs may be any of Kabat, Chothia, combined CDRs, or combinations thereof. In the present invention, the CDRs are all defined by the Chothia numbering system, unless the CDR is specifically defined by a certain numbering system.

As used herein, the term “framework region” or “FR” residues refers to those amino acid residues in the variable region of an antibody other than the CDR residues as defined above.

As used herein, the term “germline antibody gene” is an immunoglobulin sequence encoded by a non-lymphocyte, which has not undergone the processes of genetic rearrangement and maturation that lead to the expression of a specific immunoglobulin. One advantage provided by various embodiments of the present invention arises from the recognition that germline antibody genes retain more of the important amino acid sequence structures characterizing the individual of an animal species than mature antibody genes. Thus, when applied therapeutically to that species, it is less likely to be recognized as a foreign substance by that species.

As used herein, the term “antigen-binding fragment” of antibody refers to a polypeptide of an antibody fragment, such as a polypeptide of a fragment of a full-length antibody, that retains the ability to specifically bind the same antigen to which the full-length antibody binds, and/or compete with the full-length antibody for specific binding to the antigen, which is also referred to as an “antigen-binding portion.” See generally, Fundamental Immunology, Ch. 7 (Paul, W., ed., 2nd ed., Raven Press, N.Y. (1989), which is hereby incorporated by reference in its entirety for all purposes. The antigen-binding fragment of antibody can be produced by recombinant DNA techniques or by enzymatic or chemical cleavage of an intact antibody. Non-limiting examples of antigen-binding fragments include camelid Ig, Ig NAR, Fab fragment, Fab′ fragment, F(ab′)₂ fragment, F(ab′)₃ fragment, Fd, Fv, scFv, di-scFv, (scFv)₂, minibody, bifunctional antibody, trifunctional antibody, tetrafunctional antibody, disulfide stabilized Fv protein (“dsFv”) and single domain antibody (sdAbs, nanobody), and such polypeptides comprising at least a portion of an antibody sufficient to confer specific antigen-binding ability to the polypeptide. The engineered antibody variants are reviewed by Holliger et al., 2005; Nat Biotechnol, 23: 1126-1136.

As used herein, the term “camelid Ig” or “camel VHH” refers to the smallest known antigen-binding unit of a heavy chain antibody (Koch-Nolte et al., FASEB J., 21:3490-3498 (2007)). “Heavy chain antibody” or “camel antibody” refers to an antibody containing two VH domains and no light chain (Riechmann L. et al., J. Immunol. Methods, 231:25-38 (1999); WO94/04678; WO94/25591; U.S. Pat. No. 6,005,079).

As used herein, the term “IgNAR” or “immunoglobulin novel antigen receptor” refers to a class of antibodies from the shark immune repertoire and consisting of homodimer of one variable new antigen receptor (VNAR) domain and five constant neoantigen receptor (CNAR) domains.

As used herein, the term “Fd” refers to an antibody fragment consisting of VH and CH1 domains; the term “dAb fragment” refers to an antibody fragment consisting of VH domain (Ward et al., Nature, 341:544546 (1989)); the term “Fab fragment” refers to an antibody fragment consisting of VL, VH, CL and CH1 domains; the term “F(ab′)₂ fragment” refers to an antibody fragment comprising an antibody fragment of two Fab fragments that are linked by disulfide bridge on the hinging region; the term “Fab′ fragment” refers to a fragment that is obtained by reducing the disulfide bond linking two heavy chain fragments in an F(ab′)₂ fragment, and consists of one intact light chain and a heavy chain Fd fragment (consisting of VH and CH1 domains).

As used herein, the term “Fv” refers to an antibody fragment consisting of the VL and VH domains of one-arm of an antibody. Fv fragments are generally considered to be the smallest antibody fragments capable of forming a complete antigen-binding site. It is generally believed that the six CDRs confer antigen-binding specificity to an antibody. However, even a variable region (e.g., an Fd fragment, which contains only three antigen-specific CDRs) is able to recognize and bind an antigen, albeit with possibly lower affinity than the intact binding site.

As used herein, the term “Fc” refers to an antibody fragment formed by linking the second and third constant regions of the first heavy chain of antibody with the second and third constant regions of the second heavy chain through disulfide bonds. The Fc fragment of an antibody has many different functions, but is not involved in antigen binding.

As used herein, the term “scFv” refers to a single polypeptide chain comprising VL and VH domains, wherein the VL and VH are connected by a linker (see, for example, Bird et al., Science, 242:423-426 (1988); Huston et al., Proc. Natl. Acad. Sci. USA 85:5879-5883 (1988); and Pluckthun, The Pharmacology of Monoclonal Antibodies, Vol. 113, Eds. Roseburg and Moore, Springer-Verlag, New York, pp. 269-315 (1994)). Such scFv molecules can have the general structure: NH₂-VL-linker-VH—COOH or NH₂-VH-linker-VL-COOH. Suitable prior art linkers consist of repeated GGGGS amino acid sequences or variants thereof. For example, a linker with the amino acid sequence (GGGGS)₄ can be used, while variants thereof are also usable (Holliger et al. (1993), Proc. Natl. Acad. Sci. USA 90: 6444-6448). Other linkers useful in the present invention are described by Alfthan et al. (1995), Protein Eng. 8:725-731; Choi et al. (2001), Eur. J. Immunol. 31:94-106; Hu et al. (1996), Cancer Res. 56:3055-3061; Kipriyanov et al. (1999), J. Mol. Biol. 293:41-56; and Roovers et al. (2001), Cancer Immunol. In some cases, a disulfide bond may also exist between the VH and VL of scFv. In certain embodiments, the VH and VL domains can be positioned relative to each other in any suitable arrangement, for example, a scFv comprising NH₂-VH—VH—COOH, NH₂-VL-VL-COOH. The scFv can form any engineering possible structure: single chain antibody (scFv), tandem single chain antibody (tandem di-scFv), bifunctional antibody, trifunctional antibody, tetrafunctional antibody, disulfide-stabilized Fv protein, camel Ig, IgNAR, etc. In certain embodiments of the present invention, the scFv can form a di-scFv, which refers to an antibody formed by two or more individual scFvs in tandem connection. In certain embodiments of the present invention, the scFv can form (scFv)₂, which refers to an antibody formed by two or more individual scFvs in parallel connection.

As used herein, the term “bifunctional antibody” refers to an antibody fragment having two antigen-binding sites, in which the fragment comprises a heavy chain variable domain (VH) that is linked to a light chain variable domain (VL) in the same polypeptide chain (VH-VL). By using a linker that is too short to allow pairing between the two domains on the same chain, the domains are forced to pair with the complementary domains of the other chain, and two antigen binding sites are generated. The bifunctional antibody can be bivalent or bispecific. Bifunctional antibodies are more fully described, for example, in EP 404,097; WO 1993/01161; Hudson et al, Nat. Med., 9:129-134 (2003); and Hollinger et al, PNAS USA 90:6444-6448 (1993). Trifunctional antibodies and tetrafunctional antibodies are also described in Hudson et al., Nat. Med., 9:129-134 (2003).

As used herein, the term “single-domain antibody (sdAb)” has the meaning commonly understood by those of skill in the art, which refers to an antibody fragment composed of a single monomeric variable antibody domain (e.g., a single heavy chain variable region), which retains the ability to specifically bind to the same antigen bound by the full-length antibody (Holt, L. et al., Trends in Biotechnology, 21(11):484-490). Single-domain antibody is also known as nanobody.

Each of the aforementioned antibody fragments retains the ability to specifically bind to the same antigen bound by the full-length antibody, and/or compete with the full-length antibody for specific binding to the antigen.

An antigen-binding fragment of antibody (e.g., an antibody fragment described above) can be obtained from a given antibody (e.g., the antibody provided by the present invention) by using conventional techniques known to those of skill in the art (e.g., recombinant DNA techniques or enzymatic or chemical fragmentation methods), and the antigen-binding fragment of antibody is screened for specificity in the same manner as being used for the intact antibody.

Herein, unless the context clearly dictates otherwise, when the term “antibody” is referred to, it includes not only the intact antibody but also the antigen-binding fragments of the antibody.

As used herein, the terms “monoclonal antibody”, “McAb”, “mAb” have the same meaning and are used interchangeably, and refer to an antibody from a population of highly homogeneous antibody molecules or a fragment of the antibody, that is, a population of identical antibody molecules, except for natural mutations that may occur spontaneously. Monoclonal antibody is highly specific for a single epitope on an antigen. Polyclonal antibody is relative to monoclonal antibody, and generally comprises at least two or more different antibodies that generally recognize different epitopes on an antigen. Furthermore, the modifier “monoclonal” only indicates that the antibody is characterized as being obtained from a population of highly homologous antibodies and should not be construed as requiring any particular method to prepare the antibody.

The monoclonal antibody of the present invention can be prepared by a variety of techniques, such as hybridoma technology (see, for example, Kohler et al. Nature, 256:495, 1975), recombinant DNA technology (see, for example, U.S. Pat. No. 4,816,567), or phage display antibody library technology (see, for example, Clackson et al. Nature 352:624-628, 1991, or Marks et al. J. Mol. Biol. 222:581-597, 1991).

For example, the monoclonal antibody can be prepared as follows. Firstly, mice or other suitable host animals are immunized with an immunogen (added with adjuvant if necessary). The immunogen or adjuvant is usually injected subcutaneously at multiple points or intraperitoneally. The immunogen can be pre-conjugated to certain known proteins, such as serum albumin or soybean trypsin inhibitor, to enhance the immunogenicity of the antigen in the host. The adjuvant may be Freund's adjuvant or MPL-TDM or the like. After the animal is immunized, lymphocytes that secrete antibodies that specifically bind to the immunogen will be produced. In addition, lymphocytes can also be obtained by in vitro immunization. Lymphocytes of interest are collected and fused with myeloma cells using a suitable fusion agent, such as PEG, to obtain hybridoma cells (Goding, Monoclonal Antibodies: Principles and Practice, pp. 59-103, Academic Press, 1996). The hybridoma cells prepared above can be inoculated into a suitable culture medium for growth, and the culture medium preferably contains one or more substances capable of inhibiting the growth of unfused, parental myeloma cells. For example, in parental myeloma cells lacking hypoxanthine guanine phosphotransferase (HGPRT or HPRT), the addition of substances such as hypoxanthine, aminopterin, and thymine (HAT medium) to the culture medium will inhibit the growth of HGPRT-defective cells. The preferred myeloma cells should have the characteristics of high fusion rate, stable antibody secretion ability, and sensitivity to HAT medium. Among them, preferred myeloma cells are murine myeloma cells, such as MOP-21 or MC-11 mouse tumor-derived cell strains (THE Salk Institute Cell Distribution Center, San Diego, Calif. USA), and SP-2/0 or X63-Ag8-653 cell strain (American Type Culture Collection, Rockville, Md. USA). In addition, there are also studies reporting that human monoclonal antibodies were prepared using human myeloma and human-murine heteromyeloma cell strains (Kozbor, J. Immunol., 133: 3001 (1984); Brodeur et al., Monoclonal Antibody Production Techniques and Applications, pp. 51-63, Marcel Dekker, Inc., New York, 1987). The culture medium of growing hybridoma cells is used to detect the production of monoclonal antibodies against specific antigens. Methods for determining the binding specificity of monoclonal antibodies produced by hybridoma cells include, for example, immunoprecipitation or in vitro binding assays, such as radioimmunoassay (RIA), enzyme-linked immunosorbent assay (ELISA). For example, the affinity of mAbs can be determined using the Scatchard assay described by Munson et al., Anal. Biochem. 107: 220 (1980). After the specificity, affinity, and reactivity of the antibody produced by the hybridoma have been determined, the cell strain of interest can undergo subcloning by the standard limited dilution method described in (Goding, Monoclonal Antibodies: Principles and Practice, pp. 59-103, Academic Press, 1996). The suitable medium can be DMEM or RPMI-1640 and the like. In addition, hybridoma cells can also grow in animals in the form of ascites tumors. Using traditional immunoglobulin purification methods, such as protein A agarose gel, hydroxyapatite chromatography, gel electrophoresis, dialysis or affinity chromatography, the monoclonal antibody secreted by subcloned cells can be purified from cell culture medium, ascites or serum.

The monoclonal antibody can also be obtained by genetic engineering recombinant technology. Using nucleic acid primers that specifically bind to the heavy chain and light chain genes of the monoclonal antibody to perform PCR amplification, the DNA molecules encoding the heavy chain and light chain genes of the monoclonal antibody can be isolated from the hybridoma cells. The obtained DNA molecule is inserted into an expression vector, then used to transfect a host cell (e.g., E. coli cell, COS cell, CHO cell, or other myeloma cell that does not produce immunoglobulin), and after being cultured under suitable conditions, the recombinantly expressed antibody of interest can be obtained.

The antibody can be purified by well-known techniques, such as affinity chromatography using protein A or protein G. Subsequently or alternatively, the specific antigen (the target molecule recognized by the antibody) or its epitope can be immobilized on a column, and the immunospecific antibody can be purified by immunoaffinity chromatography. The purification of immunoglobulin can be referred to, for example, D. Wilkinson (The Scientist, published by The Scientist, Inc., Philadelphia Pa., Vol. 14, No. 8 (Apr. 17, 2000), pp. 25-28).

As used herein, the term “chimeric antibody” refers to an antibody in which a part of light chain or/and heavy chain is derived from an antibody (which may be derived from a particular species or belong to a specific antibody class or subclass), and another part of the light chain or/and heavy chain is derived from another antibody (which may be derived from the same or different species or belong to the same or different antibody class or subclass), but in any event, it still retains binding activity to the target antigen (U.S. Pat. No. 4,816,567 to Cabilly et al; Morrison et al, Proc. Natl. Acad. Sci. USA, 81:6851-6855 (1984)). For example, the term “chimeric antibody” can include such an antibody (e.g., human-murine chimeric antibody) in which the heavy chain and light chain variable regions of the antibody are derived from a first antibody (e.g., a murine antibody) and the heavy chain and light chain variable regions are derived from a second antibody (e.g., a human antibody).

As used herein, the term “humanized antibody” refers to a genetically engineered non-human antibody, of which the amino acid sequence has been modified to increase homology to the sequence of a human antibody. Typically, all or part of the CDRs of a humanized antibody are derived from a non-human antibody (a donor antibody), and all or part of the non-CDR regions (e.g., variable FR and/or constant regions) are derived from a human immunoglobulin (a receptor antibody). Humanized antibodies generally retain the expected properties of the donor antibody, including, but not limited to, antigen specificity, affinity, reactivity, and the like. The donor antibody can be a mouse, rat, rabbit, or non-human primate (e.g., cynomolgus monkey) antibody with the expected properties (e.g., antigen specificity, affinity, reactivity, etc.). In the present application, the expected properties of the antibody of the present invention include the ability to specifically recognize/bind to BCMA, in particular human BCMA.

Humanized antibody can retain the expected properties of a non-human donor antibody (e.g., murine antibody), and can effectively reduce the immunogenicity of the non-human donor antibody (e.g., murine antibody) in a human subject, so that it is particularly advantageous. However, due to matching issues between the CDRs of the donor antibody and the FRs of the receptor antibody, the expected properties (e.g., antigen specificity, affinity, reactivity, ability to increase immune cell activity, and/or ability to enhance the immune response) of the humanized antibody are generally lower than those of the non-human donor antibody (e.g., murine antibody).

Thus, although the humanization of antibodies has been intensively studied and some progress has been made by researchers in the art (see, for example, Jones et al., Nature, 321:522-525 (1986); Reichmann et al., Nature, 332:323-329 (1988); Presta, Curr. Op. Struct. Biol., 2:593-596 (1992); and Clark, Immunol. Today 21: 397-402 (2000)), there is still lack of a detailed guidance in the art about how to fully humanize a certain donor antibody, so that the humanized antibody produced has the highest possible degree of humanization, and can retain the expected properties of the donor antibody as much as possible. Technicians in the art have to explore, study and modify a specific donor antibody, and make a lot of creative efforts to obtain a humanized antibody that has a high degree of humanization (e.g., a humanization degree of at least 75%, at least 80%, at least 85%, at least 90%, at least 91%, at least 92%, at least 93%, at least 94%, at least 95%, at least 96%, at least 97%, at least 98%, at least 99%), and retains the expected properties of the specific donor antibody.

In the present invention, in order for the humanized antibody to retain as much as possible the properties of the donor antibody (including, for example, antigen specificity, affinity, reactivity, ability to enhance immune cell activity and/or ability to enhance immune response), the framework regions (FRs) of the humanized antibody of the present invention may contain both the amino acid residues of the human receptor antibody and the amino acid residues of the corresponding non-human donor antibody.

The chimeric antibody or humanized antibody of the present invention can be prepared according to the sequence of the murine monoclonal antibody prepared above. The DNA encoding the heavy and light chains can be obtained from the target murine hybridoma and engineered to contain non-murine (e.g., human) immunoglobulin sequences using standard molecular biology techniques.

In order to prepare the chimeric antibody, murine immunoglobulin variable regions can be linked to human immunoglobulin constant regions using methods known in the art (see, for example, U.S. Pat. No. 4,816,567 to Cabilly et al.). For example, the DNA encoding VH is operably linked to another DNA molecule encoding the heavy chain constant region to obtain a full-length heavy chain gene. The sequences of human heavy chain constant region genes are known in the art (see, for example, Kabat, E. A. et al. (1991), Sequences of Proteins of Immunological Interest, Fifth Edition, U.S. Department of Health and Human Services, NIH Publication No. 91-3242), and the DNA fragments containing these regions can be obtained by standard PCR amplification. The heavy chain constant region may be IgG1, IgG2, IgG3, IgG4, IgA, IgE, IgM or IgD constant region, but is generally preferably IgG1 or IgG4 constant region. For example, the DNA encoding VL is operably linked to another DNA molecule encoding the light chain constant region CL to obtain a full-length light chain gene (as well as a Fab light chain gene). The sequences of human light chain constant region genes are known in the art (see, for example, Kabat, E. A. et al. (1991), Sequences of Proteins of Immunological Interest, Fifth Edition, U.S. Department of Health and Human Services, NIH Publication No. 91-3242), and the DNA fragments containing these regions can be obtained by standard PCR amplification. The light chain constant region may be κ or λ constant region, but generally and preferably κ constant region.

In order to prepare the humanized antibody, murine CDRs can be grafted into human framework sequences using methods known in the art (see U.S. Pat. No. 5,225,539 to Winter; U.S. Pat. Nos. 5,530,101, 5,585,089, 5,693,762 and 6,180,370 to Queen et al; and Lo, Benny, K. C., editor, in Antibody Engineering: Methods and Protocols, volume 248, Humana Press, New Jersey, 2004). Alternatively, transgenic animals that are capable of producing complete human antibody repertoire without producing endogenous immunoglobulins after immunization, can also be utilized. For example, it has been reported that the homozygous deletion of the antibody heavy chain joining region (JH) gene in chimeric and germ-line mutant mice results in complete inhibition of endogenous antibody production, and transfer of the human germ-line immunoglobulin gene array in such germ-line mutant mice will result in the production of human antibodies upon antigen challenge (see, for example, Jakobovits et al., 1993, Proc. Natl. Acad. Sci. USA 90: 2551; Jakobovits et al., 1993, Nature 362: 255-258; Bruggermann et al., 1993, Year in Immunology 7: 33; and Duchosal et al., 1992, Nature 355: 258). Non-limiting examples of the above-mentioned transgenic animal include HuMAb mice (Medarex, Inc.) which comprises human immunoglobulin gene miniloci encoding unrearranged human heavy chains (μ and γ) and κ light chain immunoglobulin sequences, and a targeted mutation that inactivates endogenous μ and κ chain loci (see, for example, Lonberg et al. (1994) Nature 368 (6474): 856-859); or “KM Mouse™” which carries a human heavy chain transgene and human light chain transchromosome (see: patent application WO02/43478). Other methods of humanizing antibodies include phage display technology (Hoogenboom et al., 1991, J. Mol. Biol. 227: 381; Marks et al., J. Mol. Biol. 1991, 222: 581-597; Vaughan et al., 1996, Nature Biotech 14: 309).

As used herein, the term “degree of humanization” is an indicator used to evaluate the number of non-human amino acid residues in a humanized antibody. The degree of humanization of the humanized antibody can be assessed, for example, by predicting the homology of the variable region sequence to the human V domain with the Domain Gap Align of IMGT website.

As used herein, the expression “specifically binding” or “specifically targeting” refers to a non-random binding reaction between two molecules, such as between an antibody and an antigen to which it targets. The strength or affinity of a specific binding interaction can be expressed in terms of the equilibrium dissociation constant (K_{D}) for that interaction. In the present invention, the term “K_{D}” refers to the dissociation equilibrium constant of a specific antibody-antigen interaction, which is used to describe the binding affinity between an antibody and an antigen. The smaller the equilibrium dissociation constant, the tighter the antibody-antigen binding and the higher the affinity between the antibody and the antigen.

The specific binding property between two molecules can be determined by using methods well known in the art. One of the methods involves measuring the rate of formation and dissociation of antigen binding site/antigen complex. Both the “association rate constant” (ka or kon) and the “dissociation rate constant” (kdis or koff) can be calculated from the concentrations and the actual rates of association and dissociation (see, Malmqvist M, Nature, 1993, 361:186-187). The ratio of kdis/kon is equal to the dissociation constant K_{D} (see, Davies et al., Annual Rev Biochem, 1990; 59:439-473). The values of K_{D}, kon and kdis can be measured by any valid methods. In certain embodiments, the dissociation constant can be measured in Biacore using surface plasmon resonance (SPR). In addition, bioluminescence interferometry or Kinexa can be used to measure the dissociation constant.

As used herein, the term “identity” refers to the match degree between two polypeptides or between two nucleic acids. When two sequences for comparison have the same monomer sub-unit of base or amino acid at a certain site (e.g., each of two DNA molecules has an adenine at a certain site, or each of two polypeptides has a lysine at a certain site), the two molecules are identical at the site. The percent identity between two sequences is a function of the number of identical sites shared by the two sequences over the total number of sites for comparison×100. For example, if 6 of 10 sites of two sequences are matched, these two sequences have an identity of 60%. For example, DNA sequences: CTGACT and CAGGTT share an identity of 50% (3 of 6 sites are matched). Generally, the comparison of two sequences is conducted in a manner to produce maximum identity. Such alignment can be conducted by using a computer program such as Align program (DNAstar, Inc.) which is based on the method of Needleman, et al. (J. Mol. Biol. 48:443-453, 1970). The percent identity between two amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “conservative substitution” refers to an amino acid substitution that does not adversely affect or alter the intended properties of the protein/polypeptide comprising the amino acid sequence. For example, a conservative substitution can be introduced by standard techniques known in the art such as site-directed mutagenesis and PCR-mediated mutagenesis. Conservative amino acid substitutions include substitutions of amino acid residues with amino acid residues that have similar side chains, for example, substitutions with residues that are physically or functionally similar to the corresponding amino acid residues (e.g., have similar size, shape, charge, chemical properties, including the ability to form covalent bond or hydrogen bond, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β branched side chains (e.g., threonine, valine, isoleucine), and aromatic side chains (e.g., tyrosine, phenylalanine, tryptophan, histidine). Therefore, it is preferred to substitute the corresponding amino acid residue with another amino acid residue from the same side chain family. Methods for identifying conservative substitutions of amino acids are well known in the art (see, for example, Brummell et al., Biochem. 32:1180-1187 (1993); Kobayashi et al. Protein Eng. 12(10):879-884 (1999) and Burks et al. Proc. Natl Acad. Set USA 94:412-417 (1997), which is incorporated herein by reference).

The twenty conventional amino acids referred to herein have been complied for conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, the terms “polypeptide” and “protein” have the same meaning and are used interchangeably. And in the present invention, amino acids are generally represented by one-letter or three-letter abbreviations well known in the art. For example, alanine can be represented by A or Ala.

As used herein, the term “cytotoxic agent” includes any agent that is detrimental to a cell (e.g., killing cell), and its examples include chemotherapeutic drug, bacterial toxin, plant toxin, or radioisotope, and the like.

As used herein, the terms “nucleic acid molecule”, “nucleotide sequence”, “polynucleotide” refer to messenger RNA (mRNA), RNA, genomic RNA (gRNA), positive-strand RNA (RNA(+)), negative-strand RNA (RNA(−)), genomic DNA (gDNA), complementary DNA (cDNA), or recombinant DNA. The polynucleotides include single-stranded and double-stranded polynucleotides.

As used herein, the term “vector” refers to a nucleic acid delivery vehicle into which a polynucleotide can be inserted. The vector may include sequences that replicate directly and autonomously in the cell, or may include sequences sufficient to allow integration into the DNA of the host cell. When the vector can express the protein encoded by the inserted polynucleotide, the vector is called an expression vector. The vector can be introduced into a host cell by transformation, transduction or transfection, so that the genetic material elements carried by it can be expressed in the host cell. The vector is well known to those skilled in the art and includes, but is not limited to: plasmid; phagemid; cosmid; artificial chromosome such as yeast artificial chromosome (YAC), bacterial artificial chromosome (BAC) or P1 derived artificial chromosome (PAC); phage such as λ phage or M13 phage and viral vector. Non-limiting examples of viral vector include, retrovirus (including lentivirus), adenovirus, adeno-associated virus, herpesvirus (e.g., herpes simplex virus), poxvirus, baculovirus, papillomavirus, papovavirus (e.g., SV40). A vector may contain a variety of elements that control expression, including, but not limited to, promoter sequence, transcription initiation sequence, enhancer sequence, selection element, and reporter gene. Additionally, the vector may also contain an origin of replication site.

As used herein, “episomal” in the term “episomal vector” means that the vector is capable of replicating without integrating into the chromosomal DNA of the host and will not be gradually lost in dividing host cells, and also means that the vector is extrachromosomally or episomally replicated.

As used herein, the term “viral vector” is used broadly to refer to a nucleic acid molecule (e.g., a transfer plasmid) that includes a virus-derived nucleic acid element that typically facilitates transfer or integration of the nucleic acid molecule into the genome of a cell, or a virus particle that mediates nucleic acid transfer. In addition to nucleic acids, viral particles will typically include various viral components, and sometimes host cell components.

The term “viral vector” can refer to a virus or viral particle capable of transferring a nucleic acid into a cell, or to the transferred nucleic acid itself. The viral vector and transfer plasmid contain structural and/or functional genetic elements derived primarily from the virus.

As used herein, the term “retroviral vector” refers to a viral vector or plasmid containing structural and functional genetic elements or portions thereof derived primarily from a retrovirus.

As used herein, the term “lentiviral vector” refers to a viral vector or plasmid containing structural and functional genetic elements or portions thereof (including LTR) derived primarily from a lentivirus. In certain embodiments, the terms “lentiviral vector”, “lentiviral expression vector” may be used to refer to a lentiviral transfer plasmid and/or infectious lentiviral particle. Where elements (e.g., cloning site, promoter, regulatory element, heterologous nucleic acid, etc.) are referred to herein, it should be understood that the sequences of these elements are present in the lentiviral particle of the present invention in RNA form, and in the DNA plasmid of the present invention in DNA form.

As used herein, an “integration-deficient” retrovirus or lentivirus refers to a retrovirus or lentivirus that has an integrase that is unable to integrate the viral genome into the genome of a host cell. In certain embodiments, the integrase protein is mutated to specifically reduce its integrase activity. The integration-deficient lentiviral vector can be obtained by modifying the pol gene encoding an integrase protein to generate a mutant pol gene encoding an integration-deficient integrase. The integration-deficient viral vectors have been described in patent application WO 2006/010834, which is incorporated herein by reference in its entirety.

As used herein, the term “host cell” refers to a cell that can be used for introduction of a vector, including, but not limited to, prokaryotic cell such as Escherichia coli or Bacillus subtilis, fungal cell such as yeast cell or Aspergillus, insect cell such as S2 fruit fly cell or Sf9, or animal cell such as fibroblast, CHO cell, COS cell, NSO cell, HeLa cell, BHK cell, HEK 293 cell or human cell, immune cell (e.g., T lymphocyte, NK cell, monocyte, macrophage or dendritic cell, etc.). The host cell can include a single cell or a population of cells.

In certain embodiments, the host cell may comprise a cell that undergoes electroporation, transfection, or transduction in vivo, ex vivo or in vitro, with the isolated nucleic acid molecule of the present invention or a vector comprising the nucleic acid molecule (e.g., the vector of the present invention). In such embodiments, the host cell is preferably an immune cell.

In certain embodiments, the host cell may comprise a cell that undergoes electroporation, transfection, or transduction in vivo, ex vivo or in vitro, with the isolated nucleic acid molecule of the present invention or a vector comprising the nucleic acid molecule (e.g., the vector of the present invention).

As used herein, the term “chimeric antigen receptor” or “CAR” refers to a recombinant polypeptide construct comprising at least one antigen-binding domain, a spacer domain, a transmembrane domain, and a cytoplasmic signaling domain (also referred to herein as a “intracellular signaling domain”), which combine the antibody-based specificity for an antigen of interest (e.g., BCMA or CD19) with an activating intracellular domain of immune effector cell to exhibit a specific immune activity targeting the cell expressing the antigen of interest (e.g., BCMA or CD19). In the present invention, the expression “CAR-expressing immune effector cell” refers to an immune effector cell that expresses CAR and have an antigen specificity determined by the targeting domain of the CAR. Methods for preparing CARs (e.g., for cancer treatment) are known in the art; see, for example, Park et al, Trends Biotechnol., 29: 550-557, 2011; Grupp et al, N Engl J Med., 368: 1509-1518, 2013; Han et al., J. Hematol. Oncol., 6: 47, 2013; PCT Patent Publications WO2012/079000, WO2013/059593; and U.S. Patent Publication 2012/0213783, all of which are incorporated herein by reference in their entirety.

As used herein, the term “CAR construct” means that it comprises independent multiple (e.g., two, three, four, or more) CARs, each CAR binds to a single antigen and individually existing on the cell surface, each CAR has antigen specificity for its respective target, and each CAR can induce an antigen-specific response. Without being bound by any theory or mechanism, the nucleotide sequences encoding these CARs are linked by the sequences encoding self-cleaving peptides, so that after the full sequence of the CAR construct is completely translated, these CARs are separately/simultaneously cleaved and released; or that one CAR is cleaved before the next CAR is translated, thereby releasing each CAR (e.g., a first CAR and a second CAR). In embodiments, such CAR construct may have two independent CARs, e.g., bicistronic CAR (BiCAR).

As used herein, the terms “extracellular antigen-binding domain”, “extracellular ligand-binding domain”, “antigen-binding fragment” and “antigen-binding domain” are used interchangeably and refer to a polypeptide capable of specifically binding to the antigen or receptor of interest. This domain will be able to interact with cell surface molecules. For example, extracellular antigen-binding domains can be selected to recognize antigens that are used as cell surface markers of target cells associated with a particular disease state.

As used herein, the term “intracellular signaling domain” refers to a protein portion that transmits effector signal/function signal and directs a cell to perform a specialized function. Thus, the intracellular signaling domain has the ability to activate at least one normal effector function of CAR-expressing immune effector cells. For example, the effector function of T cells can be cytolytic activity or helper activity, including secretion of cytokines.

As used herein, the term “primary signaling domain” refers to a protein portion that is capable of modulating primary activation of the TCR complex in a stimulatory manner or in an inhibitory manner. Primary signaling domains that act in a stimulatory manner typically contain a signaling motif known as immunoreceptor tyrosine-based activation motif (ITAM). Non-limiting examples of ITAM containing primary signaling domain particularly useful in the present invention include those derived from TCRζ, FcRγ, FcRβ, CD3γ, CD3δ, CD3ε, CD3ζ, CD22, CD79a, CD79b, and CD66d.

As used herein, the term “costimulatory signaling domain” refers to an intracellular signaling domain of a costimulatory molecule. The costimulatory molecule is a cell surface molecule other than antigen receptor or Fc receptor, which, upon binding to an antigen, provides a secondary signal required for efficient activation and function of T lymphocyte. Non-limiting examples of such costimulatory molecule include CARD11, CD2, CD7, CD27, CD28, CD30, CD40, CD54 (ICAM), CD83, CD134 (OX40), CD137 (4-1BB), CD150 (SLAMF1), CD270 (HVEM), CD278 (ICOS), DAP10.

As used herein, the term “self-cleaving peptide” refers to a class of peptides that can induce cleavage of recombinant proteins in cells, for example 2A self-cleaving peptide that is a family of peptides in length of 18 to 22 aa. The members of the 2A peptide family are frequently used in life science research. The 2A peptide family includes P2A, E2A, F2A and T2A. F2A is from foot-and-mouth disease virus 18. In some exemplary embodiments, the sequence of T2A is E G R G S L L T C G D V E E N P G P, the sequence of P2A is R A K R G S G A T N F S L L K Q A G D V E E N P G P, the sequence of E2A is Q C T N Y A L L K L A G D V E S N P G P, and the sequence of F2A is V K Q T L N F D L L K L A G D V E S N P G P.

As used herein, the term “pharmaceutically acceptable carrier and/or excipient” refers to a carrier and/or excipient that is pharmacologically and/or physiologically compatible with the subject and the active ingredient, which is well known in the art (see, for example, Remington's Pharmaceutical Sciences. Edited by Gennaro A R, 19th ed. Pennsylvania: Mack Publishing Company, 1995), and includes, but is not limited to: sterile water, physiological saline, pH adjusting agent, surfactant, adjuvant, ionic strength enhancer, diluent, agent for maintaining osmotic pressure, agent for delaying absorption, preservative. For example, the pH adjusting agent includes, but is not limited to, phosphate buffer. The surfactant includes, but is not limited to, cationic, anionic or nonionic surfactant, such as Tween-80. The ionic strength enhancer includes, but is not limited to, sodium chloride. The preservative includes, but is not limited to, various antibacterial and antifungal agents, such as p-hydroxy-benzoate ester, chlorobutanol, phenol, sorbic acid, etc. The agent for maintaining osmotic pressure includes, but is not limited to, sugar, NaCl, and their analogues. The agent for delaying absorption includes, but is not limited to, monostearate salt and gelatin. The diluent includes, but is not limited to, water, aqueous buffer (e.g., buffered saline), alcohol and polyol (e.g., glycerol), etc. The preservative includes, but is not limited to, various antibacterial and antifungal agents such as thimerosal, 2-phenoxyethanol, p-hydroxy-benzoate ester, chlorobutanol, phenol, sorbic acid, etc. The term “stabilizer” has the meaning commonly understood by those skilled in the art, and it can stabilize a desired activity of an active ingredient in a drug, includes but is not limited to sodium glutamate, gelatin, SPGA, sugar (e.g., sorbitol, mannitol, starch, sucrose, lactose, glucan, or glucose), amino acid (e.g., glutamic acid, glycine), protein (e.g., dry whey, albumin or casein) or degradation product thereof (e.g., lactalbumin hydrolyzate), etc. In certain exemplary embodiments, the pharmaceutically acceptable carrier or excipient comprises a sterile injectable liquid (e.g., aqueous or non-aqueous suspension or solution). In certain exemplary embodiments, the sterile injectable liquid is selected from the group consisting of water for injection (WFI), bacteriostatic water for injection (BWFI), sodium chloride solution (e.g., 0.9% (w/v) NaCl), dextrose solution (e.g., 5% dextrose), surfactant-containing solution (e.g., 0.01% polysorbate-20), pH buffered solution (e.g., phosphate-buffered solution), Ringer's solution, and any combination thereof.

As used herein, the term “prevention” refers to a method that is performed to prevent or delay the occurrence of a disease or disorder or symptom (e.g., tumor) in a subject. As used herein, the term “treatment” refers to a method that is performed to obtain a beneficial or desired clinical outcome. For the purposes of the present invention, the beneficial or desired clinical outcome includes, but is not limited to, alleviation of symptom, reduction in the extent of a disease, stabilization (i.e., not worsening) of a disease state, delaying or slowing the progression of a disease, amelioration or remission of a disease state, and relief of symptom (whether in part or in whole), whether detectable or undetectable. In addition, “treatment” can also refer to prolonging survival as compared to an expected survival (if not receiving treatment).

As used herein, the term “subject” refers to a mammal, such as a primate, such as a human. In certain embodiments, the term “subject” refers to a living organism in which an immune response can be elicited. In certain embodiments, the subject (e.g., human) has, or is at risk for, a B-cell related disease or condition (e.g., B-cell malignancy).

As used herein, the term “effective amount” refers to an amount sufficient to obtain, or at least partially obtain, the desired effect. For example, a prophylactically effective amount of a disease (e.g., B-cell related disease or condition) refers to an amount sufficient to prevent, arrest, or delay the onset of the disease (e.g., B-cell related disease or condition); a therapeutically effective amount of a disease refers to an amount sufficient to cure or at least partially prevent the disease and complication thereof in a patient with the disease. Determining such effective amounts is well within the ability of those skilled in the art. For example, an amount effective for therapeutic use will depend on the severity of the disease to be treated, the general state of the patient's own immune system, the patient's general conditions such as age, weight and sex, the mode of administration of drug, and additional therapy applied simultaneously, and so on.

As used herein, the term “immune cell” refers to a cell involved in an immune response, for example, involved in promoting immune effector function. Examples of immune cell include T cell (e.g., α/β T cells and γ/δ T cells), B cell, natural killer (NK) cell, natural killer T (NKT) cell, mast cells, and bone marrow-derived macrophage.

The immune cell of the present invention can be self/autologous (“self”) or non-self (“non-autologous”, such as allogeneic, syngeneic or xenogeneic). As used herein, “self” refers to cells from the same subject; “allogeneic” refers to cells of the same species that are genetically different from the cell for comparison; “syngeneic” refers to cells from different subjects that genetically same with the cell for comparison; “xenogeneic” refers to cells from species different from the cell for comparison. In a preferred embodiment, the cells of the present invention are allogeneic.

Exemplary immune cells that can be used in the CAR or CAR construct described herein include T lymphocyte. The term “T cell” or “T lymphocyte” is well known in the art and is intended to include thymocyte, immature T lymphocyte, mature T lymphocyte, resting T lymphocyte or activated T lymphocyte. The T cell may be T helper (Th) cell, such as T helper 1 (Th1) or T helper 2 (Th2) cell. The T cell can be helper T cell (HTL; CD4 T cell), CD4 T cell, cytotoxic T cell (CTL; CD8 T cell), CD4CD8 T cell, CD4CD8 T cell or any other subset of T cell. In certain embodiments, the T cell can include naive T cell and memory T cell.

Those of skill in the art will appreciate that other cells can also be used as immune cells with the CAR or CAR construct as described herein. Specifically, the immune cells also include NK cell, monocyte, macrophage or dendritic cell, NKT cell, neutrophil, and macrophage. The immune cells also include progenitor cells of immune cells, wherein the progenitor cells can be induced in vivo or in vitro to differentiate into immune cells. Thus, in certain embodiments, the immune cells include progenitor cells of immune cells, such as hematopoietic stem cells (HSCs) within a population of CD34+ cells derived from umbilical cord blood, bone marrow, or peripheral blood, which will be differentiated into mature immune cells in a subject after administration, or which can be induced in vitro to differentiate into mature immune cells.

As used herein, the term “engineered immune cell” refers to an immune cell capable of expressing any one of the antibodies or antigen-binding fragments described herein, any one of the CARs or CAR constructs described herein, or introduced therein any one of the isolated nucleic acid or vector as described herein. The CAR or CAR construct polypeptide can be synthesized in situ in the cell after the polynucleotide encoding the CAR or CAR construct polypeptide has been introduced into the cell by a variety of methods. Alternatively, the CAR or CAR construct polypeptide can be produced extracellularly and then introduced into the cell. Methods of introducing polynucleotide constructs into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct is not integrated into the genome of the cell. In other embodiments, virus-mediated methods can be used. Polynucleotides can be introduced into cells by any suitable method, such as recombinant viral vectors (e.g., retroviruses, adenoviruses), liposomes, and the like. Transient transformation methods include, for example, but are not limited to, microinjection, electroporation, or particle bombardment. The polynucleotide can be contained in a vector, such as a plasmid vector or a viral vector.

As used herein, the term “immune effector function” refers to the function or response of an immune effector cell that enhances or facilitates an immune attack on a target cell (e.g., kills the target cell, or inhibits its growth or proliferation). For example, the effector function of T cells can be cytolytic activity or helper activity, including secretion of cytokines.

As used herein, the term “B cell-related disease or condition” refers to a disease or condition involving inappropriate activity of B cell and plasma cell as well as B cell and plasma cell malignancies, including but not limited to, B cell and plasma cell malignancies or B cell- and plasma cell-associated autoimmune diseases.

As used herein, the term “B cell malignancy” includes a cancer type formed in B cells (a type of immune system cells), for example, multiple myeloma (MM) and non-Hodgkin's lymphoma (NHL). Multiple myeloma is a B-cell malignancy of mature plasma cell morphology characterized by neoplastic transformation of single clones of these types of cells. These plasma cells proliferate in the BM and can invade adjacent bones and sometimes the blood. Variants of multiple myeloma include overt multiple myeloma, smoldering multiple myeloma, plasma cell leukemia, nonsecretory myeloma, IgD myeloma, sclerosing myeloma, solitary skeletal plasmacytoma and extramedullary plasmacytoma (see, for example, Braunwald et al. (eds), Harrison's Principles of Internal Medicine, 15th ed. (McGraw-Hill 2001)). Non-Hodgkin's lymphomas cover a large group of cancers of lymphocytes (white blood cells). Non-Hodgkin's lymphomas can appear at any age and are usually characterized by larger-than-normal lymph nodes, fever, and weight loss. There are many different types of non-Hodgkin's lymphomas. For example, non-Hodgkin's lymphomas can be divided into aggressive (fast growing) and indolent (slow growing) types. Although non-Hodgkin's lymphomas can be derived from B cells and T cells, as used herein, the terms “non-Hodgkin's lymphoma” and “B-cell non-Hodgkin's lymphoma” are used interchangeably. B-cell non-Hodgkin's lymphoma (NHL) includes Burkitt lymphoma, chronic lymphocytic leukemia/small lymphocytic lymphoma (CLL/SLL), diffuse large B-cell lymphoma, follicular lymphoma, immunoblastic large cell lymphoma, precursor B lymphoblastic lymphoma, and mantle cell lymphoma. The lymphomas that develop after bone marrow or stem cell transplant are usually B-cell non-Hodgkin's lymphomas. Chronic lymphocytic leukemia (CLL) is an indolent (slow-growing) cancer that causes a slow increase in immature white blood cells (called as B lymphocytes or B cells). Cancer cells spread through the blood and bone marrow, and may also affect lymph nodes or other organs, such as the liver and spleen. CLL eventually leads to bone marrow failure. Sometimes, later in the disease, the disease is called small lymphocytic lymphoma.

The following abbreviations are used herein:

| | |
|---|---|
| CDR | Complementarity determining region in immunoglobulin variable region |
| FR | Antibody framework region: amino acid residues other than CDR residues in |
| | antibody variable regions |
| VH | Antibody heavy chain variable region |
| VL | Antibody light chain variable region |
| VK | antibody κ light chain variable region |
| IgG | Immunoglobulin G |
| Kabat | Immunoglobulin alignment and numbering system proposed by Elvin A. Kabat |
| | (see, for example, Kabat et al., Sequences of Proteins of Immunological Interest, |
| | 5th Ed. Public Health Service, National Institutes of Health, Bethesda, Md., |
| | 1991). |
| Chothia | Immunoglobulin numbering system proposed by Chothia et al., which is a |
| | classical rule for identifying CDR region boundaries based on the position of |
| | structural loop regions (see, for example, Chothia & Lesk (1987) J. Mol. Biol. |
| | 196: 901-917; Chothia et al. (1989) Nature 342: 878-883). |
| IMGT | Numbering system based on the International ImMunoGeneTics information |
| | system ® (IMGT) initiated by Lefranc et al., see: Lefranc et al, Dev. Comparat. |
| | Immunol. 27: 55-77, 2003. |
| mAb | Monoclonal antibody |
| EC₅₀ | Concentration producing 50% efficacy or binding |
| IC₅₀ | Concentration producing 50% inhibition |
| ELISA | Enzyme-linked immunosorbent assay |
| PCR | Polymerase chain reaction |
| HRP | Horseradish peroxidase |
| IL-2 | Interleukin 2 |
| IL-7 | Interleukin 7 |
| IL-15 | Interleukin 15 |
| IFN | Interferon |
| K_{D} | Equilibrium dissociation constant |
| ka or kon | Association rate constant |
| kdis or koff | Dissociation rate constant |

BENEFICIAL EFFECTS OF THE INVENTION

The present invention provides a BCMA- and CD19-targeting chimeric antigen receptor comprising the antibody or antigen-binding fragment thereof of the present invention. The immune effector cell expressing the chimeric antigen receptor of the present invention has an enhanced effector function (e.g., tumor-killing activity and cytokine-releasing activity) compared to the known BCMA- and CD19-targeting CAR-T. Therefore, the chimeric antigen receptor of the present invention is particularly suitable for preventing and/or treating a B cell-related disease or condition (e.g., B-cell malignancy and plasma cell-related malignancy, or autoimmune disease), and has great clinical value.

The embodiments of the present invention will be described in detail below with reference to the drawings and examples, but those skilled in the art will understand that the following drawings and examples are only used to illustrate the present invention, rather than limit the scope of the present invention. The various objects and advantageous aspects of the present invention will become apparent to those skilled in the art from the accompanying drawings and the following detailed description of the preferred embodiments.

### BRIEF DESCRIPTION OF THE DRAWINGS

FIG. 1 shows a schematic diagram of the binding of the constructed chimeric antigen receptor or CAR construct to tumor antigens. BCMA- and CD19-targeting chimeric antigen receptor is expressed on the surface of engineered immune cell; BCMA- and CD19-targeting CAR construct is presented on the surface of engineered immune cell in the form of co-expression of independent BCMA-targeting chimeric antigen receptor and CD19-targeting chimeric antigen receptor after cleavage via a cleavable linker.

FIG. 2 shows a schematic structural diagram of the constructed nucleic acid molecule encoding the chimeric antigen receptor or CAR construct.

FIG. 3A shows the results of IL-2 secretion levels of BCMA-positive target cells stimulated with TanCAR 01-06CAR-T, H-BCMA CAR-T and blank T.

FIG. 3B shows the results of IL-2 secretion levels of BCMA-positive target cells stimulated with TanCAR 08, 10 and blank T.

FIG. 4A shows the results of IL-2 secretion levels of CD19-positive target cells stimulated with TanCAR 01-06CAR-T, H-CD19 CAR-T and blank T.

FIG. 4B shows the results of IL-2 secretion levels of CD19-positive target cells stimulated with TanCAR 08, 10 and blank T.

FIG. 5A shows the results of IFN-γ secretion levels of BCMA-positive target cells stimulated with TanCAR 01˜06 CAR-T, H-BCMA CAR-T and blank T.

FIG. 5B shows the results of IFN-γ secretion levels of BCMA-positive target cells stimulated with TanCAR 08, 10 and blank T.

FIG. 6A shows the results of IFN-γ secretion levels of CD19-positive target cells stimulated with TanCAR 01˜06 CAR-T, H-CD19 CAR-T and blank T.

FIG. 6B shows the results of IFN-γ secretion levels of CD19-positive target cells stimulated with TanCAR 08, 10 and blank T.

FIG. 7A shows the results of in vivo inhibition of the growth of BCMA-positive target cells in solid tumor model mice by TanCAR 02/08/10 CAR-T, H-CD19 CAR-T, H-BCMA CAR-T, H-BCMA CAR-T, H-CD19 CAR-T and blank T.

FIG. 7B shows the results of in vivo inhibition of the growth of CD19-positive target cells in solid tumor model mice by TanCAR 02/08/10 CAR-T, H-CD19 CAR-T, H-BCMA CAR-T, H-BCMA CAR-T, H-CD19 CAR-T and blank T.

FIG. 8 shows the results of in vivo inhibition of the growth of BCMA- and CD19-positive target cells in hematological tumor model mice by TanCAR 02/08/10 CAR-T and blank T.

### SEQUENCE INFORMATION

The information of partial sequences involved in the present invention is provided in Table 1 below.

TABLE 1
Description of sequences

| SEQ ID NO | Description |
|---|---|
| 1 | H-BCMA VH Humanized antibody BCMA heavy chain |
| | variable region |
| 2 | H-BCMA VL Humanized antibody BCMA light chain |
| | variable region |
| 3 | H-CD19 VH Humanized antibody CD19 heavy chain |
| | variable region |
| 4 | H-CD19 VL Humanized antibody CD19 light chain |
| | variable region |
| 5 | H-BCMA VH CDR1 |
| 6 | H-BCMA VH CDR2 |
| 7 | H-BCMA VH CDR3 |
| 8 | H-BCMA VL CDR1 |
| 9 | H-BCMA VL CDR2 |
| 10 | H-BCMA VL CDR3 |
| 11 | H-CD19 VH CDR1 |
| 12 | H-CD19 VH CDR2 |
| 13 | H-CD19 VH CDR3 |
| 14 | H-CD19 VL CDR1 |
| 15 | H-CD19 VL CDR2 |
| 16 | H-CD19 VL CDR3 |
| 17 | Linker1 amino acid sequence |
| 18 | Linker2 amino acid sequence |
| 19 | Linker3 amino acid sequence |
| 20 | Linker4 amino acid sequence |
| 21 | CD8α hinge region amino acid sequence |
| 22 | CD8TM amino acid sequence |
| 23 | 4-1BB intracellular signaling domain amino acid sequence |
| 24 | CD3ζ intracellular signaling domain amino acid sequence-1 |
| 25 | Anti-murine-BCMA scFv amino acid sequence |
| 26 | FMC63 (murine-CD19 antibody) scFv amino acid sequence |
| 27 | H-BCMA scFv amino acid sequence |
| 28 | H-CD19 scFv amino acid sequence |
| 29 | H-BCMA CAR amino acid sequence |
| 30 | H-CD19 CAR amino acid sequence |
| 31 | Murine-BCMA scFv encoding nucleotide sequence |
| 32 | FMC63 (murine-CD19 antibody) scFv encoding nucleotide |
| | sequence |
| 33 | H-BCMA scFv encoding nucleotide sequence |
| 34 | H-CD19 scFv encoding nucleotide sequence |
| 35 | H-BCMA CAR encoding nucleic acid sequence |
| 36 | H-CD19 CAR encoding nucleic acid sequence |
| 37 | TanCAR 01 full-length amino acid sequence |
| 38 | TanCAR 02 full-length amino acid sequence |
| 39 | TanCAR 03 full-length amino acid sequence |
| 40 | TanCAR 04 full-length amino acid sequence |
| 41 | TanCAR 05 full-length amino acid sequence |
| 42 | TanCAR 06 full-length amino acid sequence |
| 43 | TanCAR 01 encding nucleotide sequence |
| 44 | TanCAR 02 encoding nucleotide sequence |
| 45 | TanCAR 03 encoding nucleotide sequence |
| 46 | TanCAR 04 encoding nucleotide sequence |
| 47 | TanCAR 05 encoding nucleotide sequence |
| 48 | TanCAR 06 encoding nucleotide sequence |
| 49 | N-signal peptide amino acid sequence |
| 50 | P2A amino acid sequence |
| 51 | BiCAR amino acid sequence |
| 52 | BiCAR encoding nucleotide sequence |
| 53 | Murine-CD19 VL CDR2 |
| 54 | Linker1 encoding nucleotide sequence |
| 55 | Linker2 encoding nucleotide sequence |
| 56 | Linker3 encoding nucleotide sequence |
| 57 | Linker4 encoding nucleotide sequence |
| 58 | N-signal peptide encoding nucleotide sequence |
| 59 | H-BCMA VH encoding nucleotide sequence |
| 60 | H-BCMA VL encoding nucleotide sequence |
| 61 | H-CD19 VH encoding nucleotide sequence |
| 62 | H-CD19 VL encoding nucleotide sequence |
| 63 | SFFV encoding nucleotide sequence |
| 64 | TanCAR08 amino acid sequence |
| 65 | TanCAR08 encoding nucleotide sequence |
| 66 | TanCAR10 amino acid sequence |
| 67 | TanCAR10 encoding nucleotide sequence |
| 68 | Linker 5 amino acid sequence |
| 69 | Linker5 encoding nucleotide sequence |
| 70 | IgG4Hinge amino acid sequence |
| 71 | IgG4Hinge encoding nucleotide sequence |
| 72 | CD28TM amino acid sequence |
| 73 | CD28TM encoding nucleotide sequence |
| 74 | CD3ζ intracellular signaling domain amino acid sequence-2 |
| 75 | CD3ζ intracellular signaling domain encoding nucleotide |
| | sequence-2 |
| 76 | Murine-CD19 VH |
| 77 | Murine-CD19 VL |
| 78 | Murine-CD19 VH encoding nucleotide sequence |
| 79 | Murine-CD19 VL encoding nucleotide sequence |
| 80 | FMC63 CAR full-length amino acid sequence |
| 81 | FMC63 CAR encoding nucleotide sequence |

### EXAMPLES

The present invention will now be further described with reference to the following examples, which are intended to illustrate the present invention rather than to limit it.

Unless otherwise specified, the molecular biology experimental methods and immunoassay methods used in the present invention basically refer to J. Sambrook et al., Molecular Cloning: A Laboratory Manual, 2nd Edition, Cold Spring Harbor Laboratory Press, 1989, and F. M. Ausubel et al., Refined Laboratory Guide for Molecular Biology, 3rd Edition, John Wiley & Sons, Inc., 1995; restriction enzymes were used according to the conditions recommended by the product manufacturers. Those skilled in the art appreciate that the examples describe the present invention by way of example only, and are not intended to limit the scope covered by the present invention.

### Example 1: Generation of Humanized BCMA or CD19 Monoclonal Antibody

1.1 Generation of Humanized BCMA Monoclonal Antibody

1.1.1 Generation of Anti-BCMA Murine Antibody

1) Antigen Preparation

The CDS sequences (NM_001192.2) of human BCMA were retrieved from the NCBI data, full gene synthesized, and cloned into vectors; CHO-K1 (ATCC) was resuscitated and cultured to logarithmic growth phase. The cells were infected with lentivirus to overexpress BCMA, and a CHO-K1-BCMA recombinant cell line with high expression of BCMA was constructed. The K562(ATCC)-BCMA cell line with high expression of BCMA was obtained by a similar method.

2) Immunization

The CHO-K1-BCMA cell line expressing the target protein was resuscitated, 5 to 8 Balb/C mice were taken, then the suspension of CHO-K1-BCMA cells expressing the target antigen was injected into the abdominal cavity of the mice with a single-use syringe, and the immunization was repeated for three times; booster immunization and hybridoma preparation were performed.

3) SP2/0-B Cell Fusion

Myeloma cells were resuscitated and passaged using 8-azaguanine medium, spleen cells were harvested from immunized mice, and then the SP2/0 cells and the spleen cells were fused by electrofusion. After further selection by HAT (hypoxanthine, aminopterin, and thymine), the hybridoma clones grown in the original 96-well plates were transferred to a new 96-well plate with replaced medium.

4) Hybridoma Flow Cytometry Screening

The medium supernatants were taken out from the wells, co-incubated with the CHO-K1-BCMA recombinant cell line expressing the target protein, respectively, and identified by FACS. The FACS-confirmed positive single clones of hybridoma were expanded into 24-well plates according to the cell growth density. In the 24-well plate culture stage, a part of the medium supernatant was collected and re-tested by FACS to determine that the selected hybridomas could be used for secretion of antibodies. Thereby the hybridoma cell line KLB15 was obtained, which was deposited in the China Center for Type Culture Collection (CCTCC) on Nov. 14, 2018, and had the deposit number CCTCC NO. C2018224. Then, the murine monoclonal antibody was isolated and purified from the culture supernatant of the hybridoma cell line.

1.1.2 Affinity Determination of Anti-BCMA Murine Antibody

Surface plasmon resonance (SPR) could dynamically reflect the association/dissociation rates and affinity constant of antibody-antigen interaction in real time. If the sample to be tested was an antibody, it could be performed directly using the supernatant of hybridoma cell culture. The operation process was completed on the surface plasmon resonance (SPR) biosensor (BIAcore® T200 (GE HealthCare)). In brief, the antigen (human BCMA) to be recognized by the antibody to be tested (the monoclonal antibody secreted by the hybridoma obtained in Example 1) was coupled to the surface of sensor chip CM5, then the antibody to be tested was injected, and the kinetic parameters of the antibody to be tested could be measured quickly and semi-quantitatively by directly monitoring the antibody-antigen binding process on the surface of the chip. The results showed that the murine monoclonal antibody had an association rate constant ka=4.177×10⁵ M⁻¹s⁻¹, a dissociation rate constant kdis=0.003709 s⁻¹, and a dissociation equilibrium constant K_{D}=8.881×10⁻⁹ M. The above results showed that the murine monoclonal antibody obtained in step 1.1.1 had good binding affinity to human BCMA.

1.1.3 Humanization and Affinity Determination of Anti-BCMA Murine Antibody

1) Determination of Murine Antibody Variable Region Sequence

1×10⁶ Hybridoma monoclonal cells obtained in step 1.1.1 were subjected to RNA extraction to prepare cDNA, and the VH and VL were cloned and sequenced to obtain the heavy chain variable region sequence and light chain variable region sequence of the murine BCMA antibody; the heavy chain CDRs (HCDR1, HCDR2 and HCDR3) of the antibody were as set forth in SEQ ID NOs: 5-7, respectively, and the light chain CDRs (LCDR1, LCDR2 and LCDR3) of the antibody were as set forth in SEQ ID NOs: 8-10. The above CDR sequences were defined using the Chothia numbering system, and any other CDR sequence determination method known in the art could also be used to identify the amino acid residues of the CDRs in the variable regions.

2) Design and Preparation of Humanized Antibody

According to the amino acid sequence of the heavy chain variable region and the light chain variable region of the murine BCMA monoclonal antibody, the humanization design was carried out, and the CDR sequence of the murine monoclonal antibody was retained. The involved CDR sequences were shown in Table 2.

TABLE 2
CDR sequences of humanized anti-BCMA antibodies

| Name | Sequence | SEQ ID NO: |
|---|---|---|
| H-BCMA VH CDR1 | GYTFTDY | 5 |
| H-BCMA VH CDR2 | NTETGE | 6 |
| H-BCMA VH CDR3 | SLYYGYSWFTY | 7 |
| H-BCMA VL CDR1 | RSSQSLVHSNGNTFLH | 8 |
| H-BCMA VL CDR2 | KVSNRFS | 9 |
| H-BCMA VL CDR3 | MQSTHVLT | 10 |

The above CDR sequences were defined using the Chothia numbering system, and any other CDR sequence determination method known in the art could also be used to identify the amino acid residues of the CDRs in the variable regions. According to the results of germline alignment and the results of antibody simulation, four different human antibody templates were selected for the heavy chain and light chain respectively, and back mutation could be introduced in the framework region after humanization. Thereby a heavy chain variable region (named as H-BCMA VH, of which amino acid sequence was as set forth in SEQ ID NO: 1, and of which nucleotide sequence was as set forth in SEQ ID NO: 59) and a light chain variable region sequence (named as H-BCMA VL, of which amino acid sequence was as set forth in SEQ ID NO: 2, and of which nucleotide sequence was as set forth in SEQ ID NO: 60) of humanized anti-BCMA antibody were designed and obtained. The humanized anti-BCMA antibody was prepared in the form of scFv and named as H-BCMA scFv, of which the amino acid sequence was as set forth in SEQ ID NO: 27, and the nucleotide sequence encoding H-BCMA scFv was as set forth in SEQ ID NO: 33.

1.2 Production of Murine Anti-CD19 Antibody and Humanized Anti-CD19 Antibody

The anti-CD19 antibody in this example was derived from the murine FMC63 antibody, wherein the heavy chain variable region of the murine anti-CD19 antibody (named as murine-CD19 VH, has an amino acid sequence as set forth in SEQ ID NO: 76, and an encoding nucleotide sequence as set forth in SEQ ID NO: 78); and the light chain variable region sequence (named as murine-CD19 VL, has an amino acid sequence as set forth in SEQ ID NO: 77, and an encoding nucleotide sequence as set forth in SEQ ID NO: 79).

Further, the humanization design was carried out based on the amino acid sequence of the murine antibody, wherein mutation was introduced in the murine CDR sequence. The affinity of the murine antibody and the humanized antibody was determined, and the results showed that the dissociation equilibrium constant of the murine monoclonal antibody had K_{D}=2.560×10⁻⁸ M, and the dissociation equilibrium constant of the humanized monoclonal antibody had K_{D}=2.840×10⁻⁸ M, indicating that the obtained humanized monoclonal antibody had good binding affinity to human CD19. The CDR sequences involved in the anti-CD19 antibody in this example were shown in Table 3.

TABLE 3
CDR sequences of murine and humanized anti-CD19
antibodies

| | | SEQ | | SEQ |
|---|---|---|---|---|
| | Murine | ID | Humanized | ID |
| Name | sequence | NO: | sequence | NO: |
| HCDR1 | GVSLPDYG | 11 | GVSLPDYG | 11 |
| HCDR2 | IWGSETTYYN | 12 | IWGSETTYYN | 12 |
| HCDR3 | AKHYYYGGSYAMDY | 13 | AKHYYYGGSYAMDY | 13 |
| LCDR1 | QDISKY | 14 | QDISKY | 14 |
| LCDR2 | HTSRLHSGVPS | 53 | HTSRLHSGIPD | 15 |
| LCDR3 | QQGNTLPYT | 16 | QQGNTLPYT | 16 |

The above CDR sequences were defined using the Chothia numbering system, and any other CDR sequence determination method known in the art could also be used to identify the amino acid residues of the CDRs in the variable regions. According to the results of germline alignment and the results of antibody simulation, four different human antibody templates were selected for the heavy chain and light chain respectively, and back mutation could be introduced in the framework region after humanization. Thereby a heavy chain variable region (named as H-CD19 VH, of which the amino acid sequence was as set forth in SEQ ID NO: 3, and the encoding nucleotide sequence was as set forth in SEQ ID NO: 61) and a light chain variable region sequence (named as H-CD19 VL, of which the amino acid sequence was as set forth in SEQ ID NO: 4, and the encoding nucleotide sequence was as set forth in SEQ ID NO: 62) of humanized anti-CD19 antibody were designed and obtained.

Both the murine and humanized antibodies were prepared in the form of scFv, named as murine-CD19 scFv (of which the amino acid sequence was as set forth in SEQ ID NO: 26, and the nucleotide sequence encoding the murine-CD19 scFv was as set forth in SEQ ID NO: 32) and H-CD19 scFv (of which the amino acid sequence was as set forth in SEQ ID NO: 28, and the nucleotide sequence encoding the CD19 antibody of H-CD19 scFv was as set forth in SEQ ID NO: 34) respectively.

1.3 Construction of BCMA- and CD19-Targeting Antibody or Antigen-Binding Fragment Thereof

A first antibody or antigen-binding fragment thereof (specifically binding to BCMA) and a second antigen-binding fragment (specifically binding to CD19), the first antibody has VH and/or VL and the second antibody has VH and/or VL, the VH and VL regions of the first antibody and second antibody could be positioned from N-terminal to C-terminal relative to each other in any suitable arrangement, for example, VH_{(first/second)}-VL (first/second)-VH_{(first/second)}-VL (first/second), VH_{(first/second)}-VL_{(first/second)}-VL_{(first/second)}-VH_{(first/second)}, VL_{(first/second)}-VH_{(first/second)}-VL_{(first/second)}-VH_{(first/second)} or VL (first/second)-VH_{(first/second)}-VH_{(first/second)}-VL (first/second), wherein “first/second” in parentheses indicated a choice from “first antigen-binding domain” or “second antigen-binding domain”, and the adjacent variable regions were connected by a linker.

The linker sequences of the present application were as follows:

Linker 1 has an amino acid sequence (SEQ ID NO: 17): GGGGS; its encoding nucleotide sequence (SEQ ID NO: 54): GGAGGAGGAGGAAGC.

Linker 2 has an amino acid sequence (SEQ ID NO: 18): GGGGS GGGGS GGGGS; its encoding nucleotide sequence (SEQ ID NO: 55): GGAGGAGGAGGAAGC GGAGGAGGAGGAAGC GGAGGAGGAGGAAGC.

Linker 3 has an amino acid sequence (SEQ ID NO: 19): GGGGS GGGGS GGGGS GGGGS; its encoding nucleotide sequence (SEQ ID NO: 56): GGAGGAGGAGGAAGT GGAGGAGGAGGATCCGGCGGC GGCGGCTCTGGCGGCGGCGGCAGC.

Linker 4 has an amino acid sequence (SEQ ID NO: 20): EAAAK EAAAK EAAAK; its encoding nucleotide sequence (SEQ ID NO: 57): GAGGCAGCAGCAAAGGAGGCA GCAGCCAAGGAGGCAGCAGCAAAG.

Linker 5 has an amino acid sequence (SEQ ID NO: 68): GSTSGSGKPGSGEGSTKG; its encoding nucleotide sequence (SEQ ID NO: 69): GGGTCTACTTCCGGATCAGGTAAG CCCGGCTCGGGTGAGGGCTCCACGAAGGGT.

### Example 2: Construction of BCMA- and CD19-Targeting Chimeric Antigen Receptor CAR or CAR Construct

In this example, anti-BCMA and anti-CD19 chimeric antigen receptor CAR (TanCAR) or CAR construct (BiCAR) was constructed, the CAR or CAR construct comprised a N-signal peptide, an anti-BCMA antigen-binding domain, an anti-CD19 antigen-binding domain, a spacer domain, a transmembrane domain, an intracellular signaling domain. The anti-CD19 or anti-BCMA antigen-binding domain in this example was a single-chain antibody, specifically scFv, and the antibody sequences were derived from the humanized anti-BCMA antibody and anti-CD19 antibody prepared in Example 1.

2.1 Construction of BCMA- and CD19-Targeting Chimeric Antigen Receptor CAR

The chimeric antigen receptor CAR comprised a first antigen-binding domain (specifically binding to BCMA) and a second antigen-binding domain (specifically binding to CD19), the first and the second antigen-binding domains have VH and/or VL, and the VH and VL regions of the first and second antigen-binding domains could be positioned from N-terminal to C-terminal relative to each other in any suitable arrangement, for example, VH_{(first/second)}-VL_{(first/second)}-VH_{(first/second)}-VL_{(first/second)}, VH_{(first/second)}-VL_{(first/second)}-VL_{(first/second)}-VH_{(first/second)}, VL_{(first/second)}-VH_{(first/second)}-VL_{(first/second)}-VH_{(first/second)} or VL_{(first/second)}-VH_{(first/second)}-VH_{(first/second)}-VL_{(first/second)}, wherein “first/second” in parentheses represented choice from “first antigen-binding domain” or “second antigen-binding domain”, and the adjacent variable regions were connected by a linker.

The linker sequences of the present application were as follows:

Linker 1 has an amino acid sequence (SEQ ID NO: 17): GGGGS; its encoding nucleotide sequence (SEQ ID NO: 54): GGAGGAGGAGGAAGC.

Linker 2 has an amino acid sequence (SEQ ID NO: 18): GGGGS GGGGS GGGGS; its encoding nucleotide sequence (SEQ ID NO: 55): GGAGGAGGAGGAAGC GGAGGAGGAGGAAGC GGAGGAGGAGGAAGC.

Linker 3 has an amino acid sequence (SEQ ID NO: 19): GGGGS GGGGS GGGGS GGGGS; its encoding nucleotide sequence (SEQ ID NO: 56): GGAGGAGGAGGAAGTGGA GGAGGAGGATCCGGCGGCGGCGGCTCTGGCGGCGGCGGCAGC.

Linker 4 has an amino acid sequence (SEQ ID NO: 20): EAAAK EAAAK EAAAK; its encoding nucleotide sequence (SEQ ID NO: 57): GAGGCAGCAGCAAAGGAGGCA GCAGCCAAGGAGGCAGCAGCAAAG.

Linker 5 has an amino acid sequence (SEQ ID NO: 68): GSTSGSGKPGSGEGSTKG; its encoding nucleotide sequence (SEQ ID NO: 69): GGGTCTACTTCCGGATCAGGTAAGCCC GGCTCGGGTGAGGGCTCCACGAAGGGT.

N-signal peptide of the present application has an amino acid sequence (SEQ ID NO: 49):

MALPVTALLLPLALLLHAARP;

Spacer domain CD8α has an amino acid sequence (SEQ ID NO: 21):

TTTPAPRPPTPAPTIASQPLSLRPEACRPAAGGAVHTRGLDFACD;

IgG4 hinge region (IgG4Hinge) has an amino acid sequence (SEQ ID NO: 70):

ESKYGPPCPPCP;

CD8 transmembrane domain (CD8TM) has an amino acid sequence (SEQ ID NO: 22):

IYIWAPLAGTCGVLLLSLVITLYC;

CD28 transmembrane domain (CD28TM) has an amino acid sequence (SEQ ID NO: 72):

FWVLVVVGGVLACYSLLVTVAFIIFWV;

4-1BB intracellular signaling domain has an amino acid sequence (SEQ ID NO: 23):

KRGRKKLLYIFKQPFMRPVQTTQEEDGCSCRFPEEEEGGCEL;

CD3ζ intracellular signaling domain-1 has an amino acid sequence (SEQ ID NO: 24):

RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;

CD3ζ intracellular signaling domain-2 has an amino acid sequence (SEQ ID NO: 74):

RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;

The amino acid sequence of the heavy chain variable region (H-CD19 VH) of the humanized anti-CD19 antigen-binding domain was as set forth in SEQ ID NO: 3, and the nucleotide sequence thereof was as set forth in SEQ ID NO: 61; the amino acid sequence of the light chain variable region (H-CD19 VL) was as set forth in SEQ ID NO: 4, and the nucleotide sequence thereof was as set forth in SEQ ID NO: 62;

The amino acid sequence of the heavy chain variable region (murine-CD19 VH) of the murine anti-CD19 antigen-binding domain was as set forth in SEQ ID NO: 76, and the nucleotide sequence thereof was as set forth in SEQ ID NO: 78; the amino acid sequence of the light chain variable region (murine-CD19 VL) was as set forth in SEQ ID NO: 77, and the nucleotide sequence thereof was as set forth in SEQ ID NO: 79;

The amino acid sequence of the heavy chain variable region (H-BCMA VH) of the anti-BCMA antigen-binding domain was as set forth in SEQ ID NO: 1, and the coding nucleotide sequence thereof was as set forth in SEQ ID NO: 59; the amino acid sequence of the light chain variable region (H-BCMA VL) was as set forth in SEQ ID NO: 2, and the coding nucleotide sequence thereof was as set forth in SEQ ID NO: 60;

The order of connection of the parts of chimeric antigen receptor was as follows:

H-BCMA CAR: N-signal peptide-scFv(H-BCMA)-CD8α-CD8TM-4-1BB-CD3zeta-1; the amino acid sequence of H-BCMA CAR was as set forth in SEQ ID NO: 29, and the coding nucleotide sequence thereof was as set forth in SEQ ID NO: 35;

H-CD19 CAR: N-signal peptide-scFv(H-CD19)-CD8α-CD8TM-4-1BB-CD3zeta-1; the amino acid sequence of H-CD19 CAR was as set forth in SEQ ID NO: 30, and the coding nucleotide sequence thereof was as set forth in SEQ ID NO: 36;

FMC63 CAR: N-signal peptide-scFv(murine-CD19)-CD8α-CD8TM-4-1BB-CD3zeta-1; the amino acid sequence of FMC63 CAR was as set forth in SEQ ID NO: 80, and the coding nucleotide sequence thereof was as set forth in SEQ ID NO: 81;

TanCAR 01: N-signal peptide-H-BCMA scFv(VL-linker2-VH)-linker3-H-CD19 scFv(VH-linker2-VL)-CD8α-CD8TM-4-1BB-CD3zeta-1; the amino acid sequence of TanCAR 01 was as set forth in SEQ ID NO: 37, and the coding nucleotide sequence thereof was as set forth in SEQ ID NO: 43;

TanCAR 02: N-signal peptide-H-BCMA scFv(VL-linker2-VH)-linker4-H-CD19 scFv(VH-linker2-VL)-CD8α-CD8TM-4-1BB-CD3zeta-1; the amino acid sequence of TanCAR 02 was as set forth in SEQ ID NO: 38, and the coding nucleotide sequence thereof was as set forth in SEQ ID NO: 44;

TanCAR 03: N-signal peptide-H-CD19 scFv(VH-linker2-VL)-linker3-H-BCMA scFv(VL-linker2-VH)-CD8α-CD8TM-4-1BB-CD3zeta-1; the amino acid sequence of TanCAR 03 was as set forth in SEQ ID NO: 39, and the coding nucleotide sequence thereof was as set forth in SEQ ID NO: 45;

TanCAR 04: N-signal peptide-H-CD19 scFv(VH-linker2-VL)-linker4-H-BCMA scFv(VL-linker2-VH)-CD8α-CD8TM-4-1BB-CD3zeta-1; the amino acid sequence of TanCAR 04 was as set forth in SEQ ID NO: 40, and the coding nucleotide sequence thereof was as set forth in SEQ ID NO: 46;

TanCAR 05: N-signal peptide-H-BCMA scFv(VL-linker1-VH)-linker3-H-CD19 scFv(VH-linker1-VL)-CD8α-CD8TM-4-1BB-CD3zeta-1; the amino acid sequence of TanCAR 05 was as set forth in SEQ ID NO: 41, and the coding nucleotide sequence thereof was as set forth in SEQ ID NO: 47;

TanCAR 06: N-signal peptide-H-CD19 VH-linker1-H-BCMA VL-linker3-H-BCMA VH-linker1-H-CD19 VL-CD8α-CD8TM-4-1BB-CD3zeta-1; the amino acid sequence of TanCAR 06 was as set forth in SEQ ID NO:42, and the coding nucleotide sequence thereof was as set forth in SEQ ID NO:48.

TanCAR 08: N-signal peptide-H-BCMA scFv(VL-linker2-VH)-linker3-H-CD19 scFv(VH-linker1-VL)-CD8α-CD8TM-4-1BB-CD3zeta-2; the amino acid sequence of TanCAR 08 was as set forth in SEQ ID NO: 64, and the coding nucleotide sequence thereof was as set forth in SEQ ID NO: 65;

TanCAR 10: N-signal peptide-H-BCMA scFv(VL-linker2-VH)-linker3-murine-CD19 scFv(VH-linker5-VL)-IgG4Hinge-CD28TM-4-1BB-CD3zeta-1; the amino acid sequence of TanCAR 10 was as set forth in SEQ ID NO: 66, and the coding nucleotide sequence thereof was as set forth in SEQ ID NO: 67;

The connection order of each part of each BCMA- and CD19-targeting chimeric antigen receptor CAR was shown below:

N-terminal → C-terminal, amino acid sequence of each element (SEQ ID NO:)

| | | Amino acid sequence of each element from N- | | | | | | | | | | | |
|---|---|---|---|---|---|---|---|---|---|---|---|---|---|
| | | terminal to C-terminal of the BCMA and CD19 | | | | | | | | | | | |
| | | binding domain | | | | | | | | Trans- | Intracellular | | |
| | Signal | Variable | | Variable | | Variable | | Variable | Hinge | membrane | singnaling | | |
| Nam | peptide | region | Linker | region | Linker | region | Linker | region | region | region | domain | Fu | Co |
| TanCAR 01 | 49 | 2 | 18 | 1 | 19 | 3 | 18 | 4 | 21 | 22 | 23, 24 | 37 | 43 |
| TanCAR 02 | 49 | 2 | 18 | 1 | 20 | 3 | 18 | 4 | 21 | 22 | 23, 24 | 38 | 44 |
| TanCAR 03 | 49 | 3 | 18 | 4 | 19 | 2 | 18 | 1 | 21 | 22 | 23, 24 | 39 | 45 |
| TanCAR 04 | 49 | 3 | 18 | 4 | 20 | 2 | 18 | 1 | 21 | 22 | 23, 24 | 40 | 46 |
| TanCAR 05 | 49 | 2 | 17 | 1 | 19 | 3 | 17 | 4 | 21 | 22 | 23, 24 | 41 | 47 |
| TanCAR 06 | 49 | 3 | 17 | 2 | 19 | 1 | 17 | 4 | 21 | 22 | 23, 24 | 42 | 48 |
| TanCAR 08 | 49 | 2 | 18 | 1 | 19 | 3 | 68 | 4 | 21 | 22 | 23, 74 | 64 | 65 |
| TanCAR 10 | 49 | 2 | 18 | 1 | 19 | 76 | 68 | 77 | 70 | 72 | 23, 24 | 66 | 67 |

2.2 Construction of BCMA- and CD19-Targeting Chimeric Antigen Receptor CAR Construct

The CAR construct comprised independently a first CAR (H-BCMA CAR) and a second CAR (H-CD19 CAR); the first CAR comprised a signal peptide, an anti-BCMA antibody or antigen-binding fragment thereof, a spacer domain, a transmembrane domain, and an intracellular signaling domain from the N-terminal to the C-terminal; the second CAR comprised a signal peptide, an anti-CD19 antibody or antigen-binding fragment thereof, a spacer domain, a transmembrane domain and an intracellular signaling domain from N-terminal to C-terminal. The nucleotide sequence encoding the first CAR and the nucleotide sequence encoding the second CAR were linked by the nucleotide sequence encoding the self-cleaving peptide P2A, so that when the above-mentioned nucleic acid molecule was expressed in a cell, the first CAR and the second CAR could be independently formed. The CAR construct was named as BiCAR.

The P2A amino acid sequence of the present application was SEQ ID NO: 50;

The nucleotide sequence of the promoter SFFV of the present application was SEQ ID NO: 63;

H-BCMA CAR: N-signal peptide-scFv(H-BCMA)-CD8α-CD8TM-4-1BB-CD3zeta-1; the amino acid sequence of H-BCMA CAR was as set forth in SEQ ID NO: 29, and the coding nucleotide sequence thereof was as set forth in SEQ ID NO: 35;

H-CD19 CAR: N-signal peptide-scFv(H-CD19)-CD8α-CD8TM-4-1BB-CD3zeta-1; the amino acid sequence of H-CD19 CAR was as set forth in SEQ ID NO: 30, and the coding nucleotide sequence thereof was as set forth in SEQ ID NO: 36;

The structure of the nucleic acid molecule encoding BiCAR: N-signal peptide-(H-BCMA CAR)-P2A-(H-CD19 CAR), and the coding nucleotide sequence thereof was as set forth in SEQ ID NO:52.

### Example 3: Construction and Preparation of Lentiviral Expression Vector of Chimeric Antigen Receptor (CAR)/CAR Construct

3.1 Construction of Lentiviral Plasmid

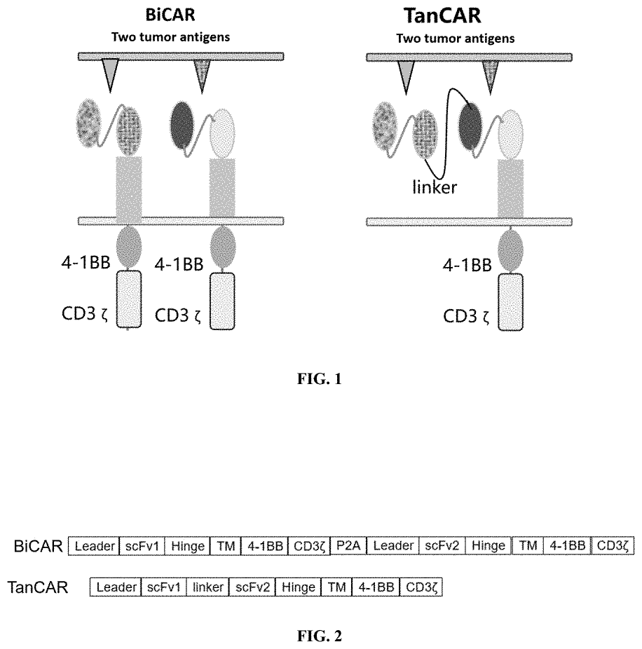

Based on the structure of the CAR or CAR construct described in Example 2, a lentiviral expression vector of CAR/CAR construct was further constructed, wherein the nucleic acid sequence encoding the CAR (TanCAR) or CAR construct (BiCAR) was subcloned into Lenti-EF1a-AT-Free vector (produced by Suzhou Aikangde Co., Ltd.); the single clones were picked for culturing and preservation, and the plasmids were finally extracted for sequencing, and the correctly sequenced bacterial liquid was used to prepare lentiviral plasmids. The structure of the chimeric antigen receptor/CAR construct constructed above was shown in FIG. 1, and the order of each element in the lentiviral expression vector encoding the CAR (TanCAR) or CAR construct (BiCAR) was shown in FIG. 2.

3.2 Virus Packaging

The mixture of the above-constructed CAR/CAR construct lentiviral plasmid and the transfection reagent was added dropwise to 293T (ATCC) cells, the culture dish was shaken gently to mix well. The culture dish was placed in a 37° C., 5% CO₂ incubator; after being cultured for 6-8 hours, the medium containing the transfection reagent was discarded and replaced with fresh complete medium. After 48 hours of continuous culture, the virus-containing medium supernatant in the culture dish was collected, filtered with a 0.45 μm filter, transferred to a centrifuge tube, and centrifuged after balancing at 20,000×g at 4° C. for 2 hours. After centrifugation, the liquid in the centrifuge tube was carefully removed by sucking in a biological safety cabinet, 500 μL of PBS buffer was added to resuspend the pellet, and the virus was stored at −80° C.

### Example 4: Preparation of CAR-T Cells

1) Isolation of Primary T Cells:

Human PBMC cells were isolated by lymphocyte separation medium (GE Healthcare), the PBMC cells were incubated with Dynabeads (Thermo) at room temperature, and subjected to enrichment by magnetic separation. T cells were resuspended in X-vivo 15 medium, and added with 10% FBS, 300 U/mL IL-2, 5 ng/mL IL-15 and 10 ng/mL IL-7 (the IL-2, IL-15, IL-7 were purchased from Novoprotein Technology Co., Ltd.), placed at 37° C., and stored in a 5% CO₂ incubator.

2) Activation of T Cells:

The cell density was adjusted to 1×10⁶ cells/mL, cytokine and antibody complex (in final concentrations of 300 U/mL IL-2, 10 ng/mL IL-7, 5 ng/mL IL-15, 500 ng/mL anti-CD3 antibody (OKT3), 2 μg/mL anti-CD28 antibody) were added to the 6-well plate, and cultured continuously for 48 hours.

3) Virus Infection:

(1) The required amount of virus was calculated according to MOI=20. The calculation formula was as follows: required virus amount (mL)=(MOI×cell number)/virus titer.

(2) The virus was rapidly rewarmed to 37° C. The calculated amount of virus was added to the 6-well plate, added with polybrene to reach a final concentration of 6 μg/mL, mixed well, and then centrifuged.

(3) After centrifugation, it was continuously cultured in a 37° C., 5% CO₂ incubator for later use.

(4) H-BCMA CAR-T, FMC63 CAR-T, H-CD19 CAR-T, TanCAR 01˜06, 08, 10 CAR-T, BiCAR-T cells were obtained.

### Example 5: Detection of Positive Rate of CAR-T Cells

The nucleic acid sequence encoding the CAR was expressed under the drive of a promoter, therefore the lentivirus-transfected T cells could be labeled with an antigen or anti-CD19 antibody and measured by flow cytometry, to reflect the expression level of the CAR on the surface of the T cells. The CAR positive rate of the CAR-T cells obtained in Example 4 was detected by the above method, and the FACS test results were shown in Table 4 below. The results showed that the CAR positive rate of all CAR-T cells was greater than 5% 48h after transduction, indicating that the CAR was successfully expressed after effector cells were transfected with lentivirus, and the chimeric antigen receptor T cells expressing BCMA-CAR and CD19-CAR were successfully constructed.

The expression of BCMA in CHO-K1-BCMA, RPMI8226 and MM.1S cells and the expression of CD19 in Nalm6 cells were detected by flow cytometry. The results were shown in Table 5, which showed that the CHO-K1-BCMA, RPMI8226 and MM.1S cells had high expression level of BCMA, the Nalm6 cells had high expression level of CD19, and thus they could be used for subsequent detection as target cells.

TABLE 4
Results of positive rate test of CAR

| Chimeric antigen | Positive rate of | Positive rate of |
|---|---|---|
| receptor | binding to BCMA | binding to CD19 |
| H-BCMA CAR | 26.94% | 0.31% |
| FMC63 CAR | 1.01% | 15.55% |
| H-CD19 CAR | 0.98% | 14.67% |
| TanCAR 01 | 11.34% | 10.6% |
| TanCAR 02 | 14.09% | 12.69% |
| TanCAR 03 | 6.07% | 8.99% |
| TanCAR 04 | 15.87% | 16.12% |
| TanCAR 05 | 14.24% | 11.78% |
| TanCAR 06 | 13.3% | 16.44% |
| TanCAR 08 | 28.54% | 24.40% |
| TanCAR 10 | 39.83% | 34.79% |

TABLE 5
BCMA and CD19 expression rates of target cells

| Cell line | BCMA expression rate | CD19 expression rate |
|---|---|---|
| CHO-K1-BCMA | 50% | / |
| MM.1S | 73.83% | / |
| RPMI8226 | 81.11% | / |
| Nalm6 | / | 88.24% |

### Example 6: Evaluation of Killing Activity of CAR-T Cells on Target Cells

The killing activity of CAR-T cells was evaluated by measuring the ability of CAR-T cells to lyse target cells and their ability to release cytokines. The specific steps were as follows: 1) Ability of CAR-T to Lyse BCMA Target Cells

The cell density of the target cell CHO-K1-BCMA-luc was adjusted to 1×10⁵/mL, and the target cell CHO-K1-BCMA-luc was inoculated in a 96-well plate according to the amount of 100 μL/well, and allowed to stand in a 5% CO₂, 37° C. incubator for 30 min. CAR-T was collected, wherein CAR-T cells of H-BCMA CAR, TanCAR 01˜06, TanCAR 08, TanCAR 10, and blank T cells without being transfected with CAR as effector cells were collected by centrifugation and resuspended with F-12K, 10% FBS medium; then they were added to 96-well plates containing CHO-K1-BCMA-luc at E/T (effector cells/target cells) ratios of 2:1, 1:1, 0.5:1, 0.25:1, 100 μL/well, and cultured in a 5% CO₂, 37° C. incubator for 18 to 24 hours. After the culturing, the plates were taken out of the incubator, added with 20 μL of fluorescence detection reagent, and detected by a microplate reader to obtain fluorescence readings.

2) Ability of CAR-T to Lyse CD19 Target Cells

The cell density of target cell Nalm6-luc was adjusted to 5×10⁴/mL, and the target cell Nalm6-luc was inoculated in a 96-well plate according to the amount of 100 μL/well, and allowed to stand for 30 min in a 5% CO₂, 37° C. incubator. CAR-T was collected, wherein CAR-T cells of H-CD19 CAR, TanCAR 01˜06, TanCAR 08, TanCAR 10, and blank T cells without being transfected with CAR as effector cells were collected by centrifugation and resuspended in RPMI 1640, 10% FBS medium; then they were added to the 96-well plates containing Nalm6-luc at E/T (effector cells/target cells) ratios of 2:1, 1:1, 0.5:1, 0.25:1, 100 μL/well, supplemented to reach a final volume of 200 μL/well, and cultured in a 5% CO₂, 37° C. incubator for 18 to 24 hours. After the culturing, the plates were taken out of the incubator, added with 20 μL of fluorescence detection reagent, and detected by a microplate reader to obtain fluorescence readings.

3) Release of Cytokines

According to the steps of 1) and 2), MM.1S-luc (or RPMI8226) and Nalm6-luc were prepared as target cells, H-BCMA CAR or H-CD19 CAR and TanCAR 01˜06, 08, 10 and blank T cells without being transfected with CAR were prepared as effector cells; then the effector cells were added to 96-well plates containing target cells at E/T (effector cells/target cells) ratio of 1:1, 100 μL/well, and supplemented to reach a final volume of 200 μL/well, and cultured in a 5% CO₂, 37° C. incubator overnight. After the culturing, the well plates were taken out of the incubator, centrifuged, and the supernatant was taken, and the release of cytokines (IL-2 and IFN-γ) was detected by ELISA kit.

The data obtained after the above processing were plotted using GraphPad 6.0.

4) Result Analysis:

The killing activity of CAR-T adopted the following formula:

Tumor cell lysis rate %=(1−(fluorescence reading with effector cells and target cells/fluorescence reading without effector cells and target cells)/(fluorescence reading with target cells only/fluorescence reading without target cells))×100%.

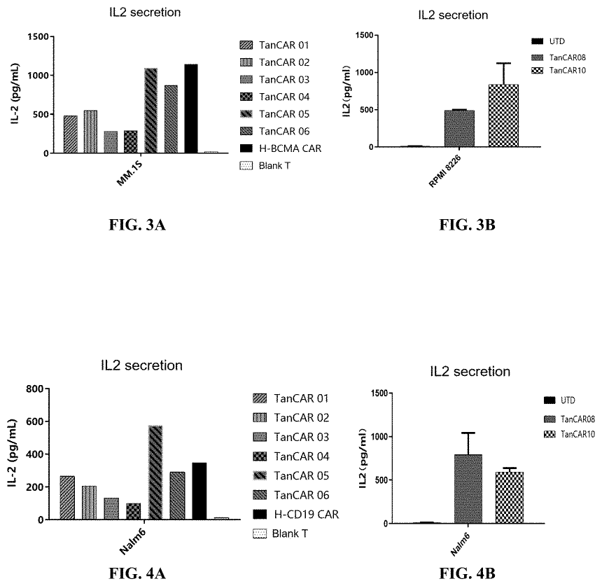

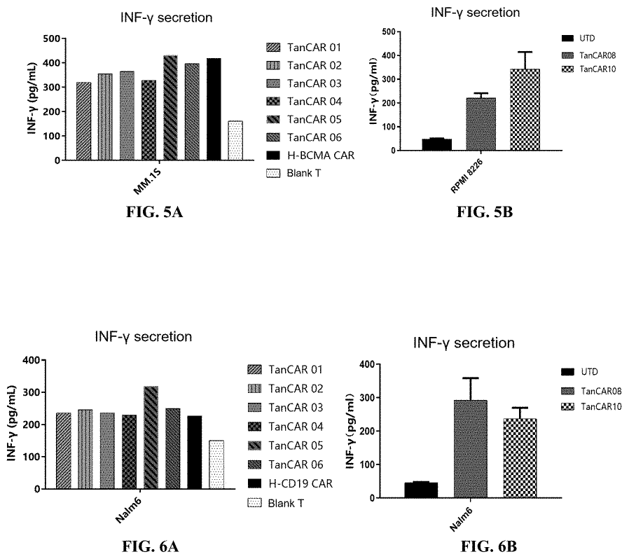

The killing test results were shown in Tables 6 and 7. The detection results of IL-2 secretion levels were shown in FIGS. 3A, 3B, 4A and 4B. The detection results of IFN-γ secretion levels were shown in FIGS. 5A, 5B, 6A and 6B.

The results showed that the constructed CAR-T could activate primary T cells and efficiently mediate the killing of targeted tumor cells by T cells (Tables 6 and 7), and caused a significant increase in cytokine secretion (FIGS. 3A, 3B, 4A, 4B, 5A, 5B, 6A, 6B). The above results showed that TanCAR 01˜06, 08, 10 all could specifically lyse tumor cells with targets of BCMA and CD19, and induce a significant increase in cytokine secretion, indicating that they could effectively kill cells expressing CD19 and/or BCMA.

TABLE 6
lysis rate of CHO-K1-BCMA-luc cells by CAR-T cells

| | Ratio of CAR-T cells to target cells (CHO-K1-BCMA) | | | |
|---|---|---|---|---|
| CAR-T cells | 0.25:1 | 0.5:1 | 1:1 | 2:1 |
| TanCAR 01 | 31.3% | 34.3% | 63.2% | 71% |
| TanCAR 02 | 24% | 35.9% | 60.7% | 59% |
| TanCAR 03 | 17.8% | 27.6% | 40.5% | 52.1% |
| TanCAR 04 | 36.4% | 52.6% | 68.9% | 84.1% |
| TanCAR 05 | 26.6% | 39.2% | 58.2% | 79.8% |
| TanCAR 06 | 31.9% | 48.3% | 78.4% | 92.9% |
| TanCAR 08 | 12.3% | 38.4% | 65.9% | / |
| TanCAR 10 | 17.1% | 36.0% | 57.9% | / |
| H-BCMA CAR | 0% | 27% | 40.7% | 77.9% |
| Blank T | 0% | 6% | 16.4% | 22% |

“/” indicates that no experiment was performed at the corresponding ratio of CAR-T cells to target cells, and there was no experimental data.

TABLE 7
lysis rate of Nalm6-luc cells by CAR-T cells

| | Ratio of CAR-T cells to target cells (Nalm6) | | | |
|---|---|---|---|---|
| CAR-T cells | 0.25:1 | 0.5:1 | 1:1 | 2:1 |
| TanCAR 01 | 46.1% | 40% | 56.8% | 80.6% |
| TanCAR 02 | 26.1% | 49.8% | 70.1% | 82.4% |
| TanCAR 03 | 54.3% | 57.7% | 72.1% | 89.1% |
| TanCAR 04 | 72.7% | 55.8% | 68.9% | 84.1% |
| TanCAR 05 | 25.8% | 42.4% | 67.7% | 91.2% |
| TanCAR 06 | 68.3% | 62.9% | 88.9% | 95.5% |
| TanCAR 08 | 65.9% | 83.8% | 92.2% | / |
| TanCAR 10 | 59.6% | 77.4% | 91.5% | / |
| H-CD19 CAR | 48% | 55% | 72.8% | 86.4% |
| Blank T | 2.6% | 0% | 0% | 0% |

“/” indicates that no experiment was performed at the corresponding ratio of CAR-T cells to target cells, and there was no experimental data.

### Example 7: In Vivo Efficacy Evaluation of CAR-T Cells

7.1 Solid Tumor Model

Animal grouping: 30 B-NDG mice were subcutaneously inoculated with the tumor cells of RPMI8226 and Nalm6 (1×10⁷/mouse) respectively, which were 6-8 weeks old female mice. The unsuccessfully modeled mice were excluded, and were randomly divided into 7 groups, wherein Group 1 (3 mice) were given H-BCMA CAR-T and H-CD19 CAR-T (BCMA+hCD19), Group 2 (3 mice) were given blank T cells (UTD), Group 3 (4 mice) were given H-BCMA CAR-T (BCMA), Group 4 (4 mice) were given H-CD19 CAR-T (hCD19), Group 5 (4 mice) were given TanCAR 02, Group 6 (4 mice) were given TanCAR 08, and Group 7 (4 mice) were given TanCAR 10. The day of reinfusion of CAR-T was recorded as P0.

Treatment:100 mg/kg cyclophosphamide was intraperitoneally injected 24 hours before administration, and CAR-T was re-infused at PO and P3 via tail vein injection at a dose of 3×10⁵/mouse. After administration, the tumor volume and body weight of mice were observed and measured regularly.

The tumor diameter was measured with a vernier caliper, and the tumor volume was calculated according to the following formula: V=0.5 a×b², wherein a and b represented the long diameter and short diameter of the tumor, respectively. The death of animals was observed and recorded every day, until P54.

The following formula was used to calculate the tumor growth inhibition rate TGI (%), which was used for evaluating the tumor suppressive efficacy of CAR-T:

TGI(%)=[1−(V_{T-end}−V_{T-start})/(V_{C-end}−V_{C-start})]×100%

wherein

V_{T-end}: mean tumor volume at the end of experiment in the treatment group

V_{T-start}: mean tumor volume at the start of administration in the treatment group

V_{C-end}: mean tumor volume at the end of experiment in the negative control group

V_{C-start}: mean tumor volume at the start of administration in the negative control group

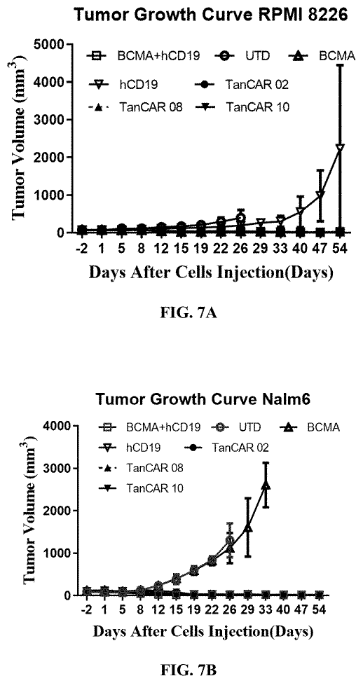

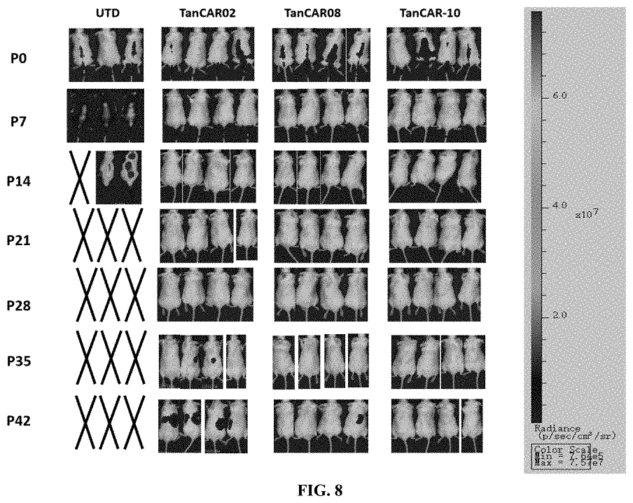

Conclusion: As shown in FIGS. 7A to 7B, from P12, TanCAR showed an inhibitory effect on the growth of BCMA and CD19 tumor cells in mice. By P54, TanCAR02, TanCAR08 and TanCAR10 completely eliminated tumor cells in vivo, and these different TanCARs showed no significant difference (the bispecific tumor inhibition rates of TanCAR02 were BCMA: 121%, CD19: 107%; the bispecific tumor inhibition rates of TanCAR08 were BCMA: 116%, CD19: 106%; the bispecific tumor inhibition rates of TanCAR10 were BCMA: 119%, CD19: 105%), and the in vivo efficacy was consistent with the efficacy of mixed refusion of BCMA CAR-T and hCD19 CAR-T (the bispecific tumor inhibition rates were BCMA: 115%, CD19: 106%, respectively).

7.2 Hematological Tumor Model

B-NDG mice (body weight 18-22 g) aged 6-8 weeks were taken and randomly divided into 4 groups; inoculation of 2×10⁶ Nalm6-BCMA-luc cells/mouse was performed through the tail vein; 7 days after inoculation, CAR-T and UTD were infused intravenously at 3×10⁶/mouse, which was recorded as P0; 1 day later (P2), refusion was performed for the second time; the weight of mice was measured by electronic balance twice a week, imaging was performed once a week, and the imaging observation was carried out until P45.

Conclusion: As shown in FIG. 8, the mice in the blank control group began to die at P14; TanCAR02, TanCAR08 and TanCAR10 could effectively kill tumor cells in mice and significantly reduce the mortality of mice. Observed until P45, the mice of the TanCAR02, TanCAR08 and TanCAR10 groups survived normally, and no tumor recurrence was found in the TanCAR10 group.

Although specific embodiments of the present invention have been described in detail, those skilled in the art will appreciate that various modifications and changes can be made to the details in light of all the teachings that have been published, and that these changes are all within the scope of the present invention. The whole of the present invention is given by the appended claims and any equivalents thereof.

1. A bispecific antibody or antigen-binding fragment thereof targeting BCMA and CD19, wherein the bispecific antibody or antigen-binding fragment thereof comprises a first antibody or antigen-binding fragment thereof targeting BCMA and a second antibody or antigen-binding fragment thereof targeting CD19, said first antibody or antigen-binding fragment thereof targeting BCMA comprises a first heavy chain variable region (VH) and/or a first light chain variable region (VL), the first VH and/or the first VL form a BCMA-binding site, and said second antibody or antigen-binding fragment thereof targeting CD19 comprises a second heavy chain variable region (VH) and/or a second light chain variable region (VL), the second VH and/or the second VL form a CD19-binding site, wherein,
the first VH comprises: a first VH CDR1 comprising an amino acid sequence as set forth in SEQ ID NO: 5 or a sequence having a substitution, deletion or addition of one or several amino acids (e.g., a substitution, deletion or addition of 1, 2 or 3 amino acids) as compared thereto; a first VH CDR2 comprising an amino acid sequence as set forth in SEQ ID NO: 6 or a sequence having a substitution, deletion or addition of one or several amino acids (e.g., a substitution, deletion or addition of 1, 2 or 3 amino acids) as compared thereto; and a first VH CDR3 comprising an amino acid sequence as set forth in SEQ ID NO: 7 or a sequence having a substitution, deletion or addition of one or several amino acids (e.g., a substitution, deletion or addition of 1, 2 or 3 amino acids) as compared thereto;
the first VL comprises: a first VL CDR1 comprising an amino acid sequence as set forth in SEQ ID NO: 8 or a sequence having a substitution, deletion or addition of one or several amino acids (e.g., a substitution, deletion or addition of 1, 2 or 3 amino acids) as compared thereto; a first VL CDR2 comprising an amino acid sequence as set forth in SEQ ID NO: 9 or a sequence having a substitution, deletion or addition of one or several amino acids (e.g., a substitution, deletion or addition of 1, 2 or 3 amino acids) as compared thereto; and a first VL CDR3 comprising an amino acid sequence as set forth in SEQ ID NO: 10 or a sequence having a substitution, deletion or addition of one or several amino acids (e.g., a substitution, deletion or addition of 1, 2 or 3 amino acids) as compared thereto;
preferably, the first VH comprises the sequence as set forth in SEQ ID NO: 1 or a variant thereof; the first VL comprises the sequence as set forth in SEQ ID NO: 2 or a variant thereof; wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived;
preferably, the substitution is a conservative substitution.

2. The bispecific antibody or antigen-binding fragment thereof according to claim 1, wherein the second VH of the second antibody or antigen-binding fragment thereof targeting CD19 comprises: a second VH CDR1 comprising an amino acid sequence as set forth in SEQ ID NO: 11 or a sequence having a substitution, deletion or addition of one or several amino acids (e.g., a substitution, deletion or addition of 1, 2 or 3 amino acids) as compared thereto; a second VH CDR2 comprising an amino acid sequence as set forth in SEQ ID NO: 12 or a sequence having a substitution, deletion or addition of one or several amino acids (e.g., a substitution, deletion or addition of 1, 2 or 3 amino acids) as compared thereto; and a second VH CDR3 comprising an amino acid sequence as set forth in SEQ ID NO: 13 or a sequence having a substitution, deletion or addition of one or several amino acids (e.g., a substitution, deletion or addition of 1, 2 or 3 amino acids) as compared thereto;
the second VL comprises: a second VL CDR1 comprising an amino acid sequence as set forth in SEQ ID NO: 14 or a sequence having a substitution, deletion or addition of one or several amino acids (e.g., a substitution, deletion or addition of 1, 2 or 3 amino acids) as compared thereto; a second VL CDR2 comprising an amino acid sequence as set forth in SEQ ID NO: 15 or 53 or a sequence having a substitution, deletion or addition of one or several amino acids (e.g., a substitution, deletion or addition of 1, 2 or 3 amino acids) as compared thereto; and a second VL CDR3 comprising an amino acid sequence as set forth in SEQ ID NO: 16 or a sequence having a substitution, deletion or addition of one or several amino acids (e.g., a substitution, deletion or addition of 1, 2 or 3 amino acids) as compared thereto;
preferably, the second VH comprises the sequence as set forth in SEQ ID NO: 3 or 76 or a variant thereof; the second VL comprises the sequence as set forth in SEQ ID NO: 4 or 77 or a variant thereof; wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived;
preferably, the substitution is a conservative substitution.

3. The bispecific antibody or antigen-binding fragment thereof according to claim 1, wherein the bispecific antibody or antigen-binding fragment thereof comprises the following domains in sequence from the N-terminal to the C-terminal:
(1) the first VH, the first VL, the second VH, the second VL;
(2) the second VH, the second VL, the first VH, the first VL;
(3) the first VL, the first VH, the second VL, the second VH;
(4) the second VL, the second VH, the first VL, the first VH;
(5) the first VH, the first VL, the second VL, the second VH;
(6) the second VH, the second VL, the first VL, the first VH;
(7) the first VL, the first VH, the second VH, the second VL;
(8) the second VL, the second VH, the first VH, the first VL;
(9) the first VL, the second VL, the second VH, the first VH;
(10) the second VL, the first VL, the first VH, the second VH;
(11) the first VH, the second VL, the second VH, the first VL;
(12) the second VH, the first VL, the first VH, the second VL;
(13) the first VL, the second VH, the second VL, the first VH;
(14) the second VL, the first VH, the first VL, the second VH;
(15) the first VH, the second VH, the second VL, the first VL; or
(16) the second VH, the first VH, the first VL, the second VL;
in any one of items (1) to (16), any adjacent variable regions are independently connected by a linker; preferably, in any one of items (1) to (16), the linker between the adjacent variable regions can be the same or different;
preferably, the first VH comprises the sequence as set forth in SEQ ID NO: 1 or a variant thereof; the first VL comprises the sequence as set forth in SEQ ID NO: 2 or a variant thereof;
wherein the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived;
preferably, the second VH comprises the sequence as set forth in SEQ ID NO: 3 or 76 or a variant thereof; the second VL comprises the sequence as set forth in SEQ ID NO: 4 or 77 or a variant thereof; wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or has a substitution, deletion or addition of one or several amino acids (e.g., a substitution, deletion or addition of 1, 2, 3, 4 or 5 amino acids) as compared to the sequence from which it is derived.

4. The bispecific antibody or antigen-binding fragment thereof according to claim 1, wherein the first antibody or antigen-binding fragment thereof targeting BCMA or the second antibody or antigen-binding fragment thereof targeting CD19 is each independently selected from the group consisting of camelid Ig, IgNAR, Fab fragment, Fab′ fragment, F(ab′)₂ fragment, F(ab′)₃ fragment, Fv, single chain antibody (e.g., scFv, di-scFv, (scFv)₂), minibody, bifunctional antibody, trifunctional antibody, tetrafunctional antibody, disulfide-stabilized Fv protein (“dsFv”) and single domain antibody (sdAb, nanobody), chimeric antibody, humanized antibody, single domain antibody, bispecific antibody or multi specific antibody;
preferably, the first antibody or antigen-binding fragment thereof targeting BCMA or the second antibody or antigen-binding fragment thereof targeting CD19 is an scFv; more preferably, the BCMA-targeting scFv comprises a first VL as set forth in SEQ ID NO: 2, a linker as set forth in SEQ ID NO: 17, 18, 19, 20 or 68, a first VH as set forth in SEQ ID NO: 1; the CD19-targeting scFv comprises a second VL as set forth in SEQ ID NO: 4 or 77, a linker as set forth in SEQ ID NO: 17, 18, 19, 20 or 68, and a second VH as set forth in SEQ ID NO: 3 or 76; more preferably, the BCMA-targeting scFv has a sequence as set forth in SEQ ID NO: 25 or 27, and the CD19-targeting scFv has a sequence as set forth in SEQ ID NO: 26 or 28.

5. The bispecific antibody or antigen-binding fragment thereof according to claim 1, which further comprises a heavy chain constant region (CH) and a light chain constant region (CL);
preferably, the heavy chain constant region is selected from the group consisting of IgG, IgM, IgE, IgD and IgA;
preferably, the light chain constant region is selected from κ or λ.

6. A chimeric antigen receptor (CAR) targeting BCMA and CD19, which comprises an antigen-binding domain, a spacer domain, a transmembrane domain and an intracellular signaling domain, wherein the antigen-binding domain comprises the bispecific antibody or antigen-binding fragment thereof according to claim 1.

7. The chimeric antigen receptor (CAR) according to claim 6, wherein the chimeric antigen receptor (CAR) comprises the following domains in sequence from the N-terminal to the C-terminal:
(1) the first VH, the first VL, the second VH, the second VL, the spacer domain, the transmembrane domain, and the intracellular signaling domain;
(2) the second VH, the second VL, the first VH, the first VL, the spacer domain, the transmembrane domain, and the intracellular signaling domain;
(3) the first VL, the first VH, the second VL, the second VH, the spacer domain, the transmembrane domain, and the intracellular signaling domain;
(4) the second VL, the second VH, the first VL, the first VH, the spacer domain, the transmembrane domain, and the intracellular signaling domain;
(5) the first VH, the first VL, the second VL, the second VH, the spacer domain, the transmembrane domain, and the intracellular signaling domain;
(6) the second VH, the second VL, the first VL, the first VH, the spacer domain, the transmembrane domain, and the intracellular signaling domain;
(7) the first VL, the first VH, the second VH, the second VL, the spacer domain, the transmembrane domain, and the intracellular signaling domain;
(8) the second VL, the second VH, the first VH, the first VL, the spacer domain, the transmembrane domain, and the intracellular signaling domain;
(9) the first VL, the second VL, the second VH, the first VH, the spacer domain, the transmembrane domain, and the intracellular signaling domain;
(10) the second VL, the first VL, the first VH, the second VH, the spacer domain, the transmembrane domain, and the intracellular signaling domain;
(11) the first VH, the second VL, the second VH, the first VL, the spacer domain, the transmembrane domain, and the intracellular signaling domain;
(12) the second VH, the first VL, the first VH, the second VL, the spacer domain, the transmembrane domain, and the intracellular signaling domain;
(13) the first VL, the second VH, the second VL, the first VH, the spacer domain, the transmembrane domain, and the intracellular signaling domain;
(14) the second VL, the first VH, the first VL, the second VH, the spacer domain, the transmembrane domain, and the intracellular signaling domain;
(15) the first VH, the second VH, the second VL, the first VL, the spacer domain, the transmembrane domain, and the intracellular signaling domain; or
(16) the second VH, the first VH, the first VL, the second VL, the spacer domain, the transmembrane domain, and the intracellular signaling domain;
optionally, in any one of items (1) to (16), any adjacent variable regions are independently connected by a linker; preferably, the linker between any adjacent variable regions is each independently selected from: a polypeptide having a sequence as shown in (GGGGS)x1 or (EAAAK)x2 (x1 and x2 are independently selected from integers from 1 to 6) or a polypeptide containing a sequence as set forth in SEQ ID NO: 68; preferably, in any one of items (1) to (16), the linker between the adjacent variable regions may be the same or different.

8. The chimeric antigen receptor (CAR) according to claim 6, wherein the chimeric antigen receptor (CAR) comprises the following in sequence from the N-terminal to the C-terminal:
an antigen-binding domain comprising the first VL, the first VH, the second VH and the second VL, the spacer domain, the transmembrane domain, and the intracellular signaling domain; or
an antigen-binding domain comprising the second VH, the second VL, the first VL and the first VH, the spacer domain, the transmembrane domain, and the intracellular signaling domain; or
an antigen-binding domain comprising the second VH, the first VL, the first VH and the second VL, the spacer domain, the transmembrane domain, and the intracellular signaling domain;
wherein, any adjacent variable regions are independently connected by a linker; preferably, the linker between any adjacent variable regions is independently selected from a polypeptide with a sequence as set forth in SEQ ID NO: 17, 18, 19, 20 or 68.

9. The chimeric antigen receptor (CAR) according to claim 6, wherein,
the first VH comprises: a first VH CDR1 comprising an amino acid sequence as set forth in SEQ ID NO: 5 or a sequence having a substitution, deletion or addition of one or several amino acids (e.g., a substitution, deletion or addition of 1, 2 or 3 amino acids) as compared thereto; a first VH CDR2 comprising an amino acid sequence as set forth in SEQ ID NO: 6 or a sequence having a substitution, deletion or addition of one or several amino acids (e.g., a substitution, deletion or addition of 1, 2 or 3 amino acids) as compared thereto; and a first VH CDR3 comprising an amino acid sequence as set forth in SEQ ID NO: 7 or a sequence having a substitution, deletion or addition of one or several amino acids (e.g., a substitution, deletion or addition of 1, 2 or 3 amino acids) as compared thereto;
the first VL comprises: a first VL CDR1 comprising an amino acid sequence as set forth in SEQ ID NO: 8 or a sequence having a substitution, deletion or addition of one or several amino acids (e.g., a substitution, deletion or addition of 1, 2 or 3 amino acids) as compared thereto; a first VL CDR2 comprising an amino acid sequence as set forth in SEQ ID NO: 9 or a sequence having a substitution, deletion or addition of one or several amino acids (e.g., a substitution, deletion or addition of 1, 2 or 3 amino acids) as compared thereto; and a first VL CDR3 comprising an amino acid sequence as set forth in SEQ ID NO: 10 or a sequence having a substitution, deletion or addition of one or several amino acids (e.g., a substitution, deletion or addition of 1, 2 or 3 amino acids) as compared thereto;
the second VH comprises: a second VH CDR1 comprising an amino acid sequence as set forth in SEQ ID NO: 11 or a sequence having a substitution, deletion or addition of one or several amino acids (e.g., a substitution, deletion or addition of 1, 2 or 3 amino acids) as compared thereto; a second VH CDR2 comprising an amino acid sequence as set forth in SEQ ID NO: 12 or a sequence having a substitution, deletion or addition of one or several amino acids (e.g., a substitution, deletion or addition of 1, 2 or 3 amino acids) as compared thereto; and a second VH CDR3 comprising an amino acid sequence as set forth in SEQ ID NO: 13 or a sequence having a substitution, deletion or addition of one or several amino acids (e.g., a substitution, deletion or addition of 1, 2 or 3 amino acids) as compared thereto; and,
the second VL comprises: a second VL CDR1 comprising an amino acid sequence as set forth in SEQ ID NO: 14 or a sequence having a substitution, deletion or addition of one or several amino acids (e.g., a substitution, deletion or addition of 1, 2 or 3 amino acids) as compared thereto; a second VL CDR2 comprising an amino acid sequence as set forth in SEQ ID NO: 15 or 53 or a sequence having a substitution, deletion or addition of one or several amino acids (e.g., a substitution, deletion or addition of 1, 2 or 3 amino acids) as compared thereto; and a second VL CDR3 comprising an amino acid sequence as set forth in SEQ ID NO: 16 or a sequence having a substitution, deletion or addition of one or several amino acids (e.g., a substitution, deletion or addition of 1, 2 or 3 amino acids) as compared thereto;
preferably, the substitution is a conservative substitution.

10. (canceled)

11. The chimeric antigen receptor (CAR) according to claim 6, wherein the antigen-binding domain comprises the following in sequence from the N-terminal to the C-terminal:
(1) the first VL as set forth in SEQ ID NO: 2, the linker as set forth in SEQ ID NO: 18, the first VH as set forth in SEQ ID NO: 1, the linker as set forth in SEQ ID NO: 19, the second VH as set forth in SEQ ID NO: 3, the linker as set forth in SEQ ID NO: 18 and the second VL as set forth in SEQ ID NO: 4;
(2) the first VL as set forth in SEQ ID NO: 2, the linker as set forth in SEQ ID NO: 18, the first VH as set forth in SEQ ID NO: 1, the linker as set forth in SEQ ID NO: 20, the second VH as set forth in SEQ ID NO: 3, the linker as set forth in SEQ ID NO: 18 and the second VL as set forth in SEQ ID NO: 4;
(3) the second VH as set forth in SEQ ID NO: 3, the linker as set forth in SEQ ID NO: 18, the second VL as set forth in SEQ ID NO: 4, the linker as set forth in SEQ ID NO: 19, the first VL as set forth in SEQ ID NO: 2, the linker as set forth in SEQ ID NO: 18 and the first VH as set forth in SEQ ID NO: 1;
(4) the second VH as set forth in SEQ ID NO: 3, the linker as set forth in SEQ ID NO: 18, the second VL as set forth in SEQ ID NO: 4, the linker as set forth in SEQ ID NO: 20, the first VL as set forth in SEQ ID NO: 2, the linker as set forth in SEQ ID NO: 18 and the first VH as set forth in SEQ ID NO: 1;
(5) the first VL as set forth in SEQ ID NO: 2, the linker as set forth in SEQ ID NO: 17, the first VH as set forth in SEQ ID NO: 1, the linker as set forth in SEQ ID NO: 19, the second VH as set forth in SEQ ID NO: 3, the linker as set forth in SEQ ID NO: 17 and the second VL as set forth in SEQ ID NO: 4;
(6) the second VH as set forth in SEQ ID NO: 3, the linker as set forth in SEQ ID NO: 17, the first VL as set forth in SEQ ID NO: 2, the linker as set forth in SEQ ID NO: 19, the first VH as set forth in SEQ ID NO: 1, the linker as set forth in SEQ ID NO: 17 and the second VL as set forth in SEQ ID NO: 4;
(7) the first VL as set forth in SEQ ID NO: 2, the linker as set forth in SEQ ID NO: 18, the first VH as set forth in SEQ ID NO: 1, the linker as set forth in SEQ ID NO: 19, the second VH as set forth in SEQ ID NO: 3, the linker as set forth in SEQ ID NO:68 and the second VL as set forth in SEQ ID NO:4; or
(8) the first VL as set forth in SEQ ID NO: 2, the linker as set forth in SEQ ID NO: 18, the first VH as set forth in SEQ ID NO: 1, the linker as set forth in SEQ ID NO: 19, the second VH as set forth in SEQ ID NO: 76, the linker as set forth in SEQ ID NO:68 and the second VL as set forth in SEQ ID NO:77.

12.-15. (canceled)

16. The chimeric antigen receptor (CAR) according to claim 6, wherein the chimeric antigen receptor comprises the signal peptide, the antigen-binding domain, the spacer domain, the transmembrane domain, the intracellular signaling domain in sequence from the N-terminal to the C-terminal;
preferably, the signal peptide comprises a heavy chain signal peptide of IgG1 or a CD8α signal peptide (e.g., a sequence as set forth in SEQ ID NO: 49);
preferably, the spacer domain comprises a hinge region of CD8 (e.g., CD8α) or IgG4 (e.g., a sequence as set forth in SEQ ID NO: 21 or 70);
preferably, the transmembrane domain comprises a transmembrane region of CD8 (e.g., CD8α) or CD28 (e.g., a sequence as set forth in SEQ ID NO: 22 or 72);
preferably, the intracellular signaling domain comprises a primary signaling domain and a costimulatory signaling domain, wherein the primary signaling domain comprises an intracellular signaling domain of CD3ζ (e.g., a sequence as set forth in SEQ ID NO: 24 or 74), the costimulatory signaling domain comprises an intracellular signaling domain of CD137 (e.g., a sequence as set forth in SEQ ID NO: 23);
preferably, the chimeric antigen receptor has an amino acid sequence selected from the group consisting of: (1) an amino acid sequence as set forth in any one of SEQ ID NOs: 37-42, 64, 66; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the sequence as set forth in any one of SEQ ID NO: 37-42, 64, 66.

17. A chimeric antigen receptor (CAR) construct targeting BCMA and CD19, the CAR construct comprising independently a first CAR and a second CAR, wherein the first CAR comprises a first antibody or antigen-binding fragment thereof targeting BCMA, a spacer domain, a transmembrane domain and an intracellular signaling domain; the second CAR comprises a second antibody or antigen-binding fragment thereof targeting CD19, a spacer domain, a transmembrane domain and an intracellular signaling domain; wherein the first antibody or antigen-binding fragment thereof comprises a first VH and/or a first VL which are defined in claim 1, and the second antibody or antigen-binding fragment thereof comprises a second VH and/or a second VL, wherein:
the second VH comprises: a second VH CDR1 comprising an amino acid sequence as set forth in SEQ ID NO: 11 or a sequence having a substitution, deletion or addition of one or several amino acids (e.g., a substitution, deletion or addition of 1, 2 or 3 amino acids) as compared thereto; a second VH CDR2 comprising an amino acid sequence as set forth in SEQ ID NO: 12 or a sequence having a substitution, deletion or addition of one or several amino acids (e.g., a substitution, deletion or addition of 1, 2 or 3 amino acids) as compared thereto; and a second VH CDR3 comprising an amino acid sequence as set forth in SEQ ID NO: 13 or a sequence having a substitution, deletion or addition of one or several amino acids (e.g., a substitution, deletion or addition of 1, 2 or 3 amino acids) as compared thereto;
the second VL comprises: a second VL CDR1 comprising an amino acid sequence as set forth in SEQ ID NO: 14 or a sequence having a substitution, deletion or addition of one or several amino acids (e.g., a substitution, deletion or addition of 1, 2 or 3 amino acids) as compared thereto; a second VL CDR2 comprising an amino acid sequence as set forth in SEQ ID NO: 15 or 53 or a sequence having a substitution, deletion or addition of one or several amino acids (e.g., a substitution, deletion or addition of 1, 2 or 3 amino acids) as compared thereto; and a second VL CDR3 comprising an amino acid sequence as set forth in SEQ ID NO: 16 or a sequence having a substitution, deletion or addition of one or several amino acids (e.g., a substitution, deletion or addition of 1, 2 or 3 amino acids) as compared thereto;
preferably, the first antibody or antigen-binding fragment thereof targeting BCMA or the second antibody or antigen-binding fragment thereof targeting CD19 is an scFv; more preferably, the BCMA-targeting scFv comprises the first VL as set forth in SEQ ID NO: 2, the linker as set forth in SEQ ID NO: 17, 18, 19, 20 or 68, the first VH as set forth in SEQ ID NO: 1; the CD19-targeting scFv comprises the second VL as set forth in SEQ ID NO: 4 or 77, the linker as set forth in SEQ ID NO: 17, 18, 19, 20 or 68, the second VH as set forth in SEQ ID NO: 3 or 76; more preferably, the BCMA-targeting scFv has a sequence as set forth in SEQ ID NO: 25 or 27, and the CD19-targeting scFv has a sequence as set forth in SEQ ID NO: 26 or 28;
preferably, the spacer domain is located between the antigen-binding domain and the transmembrane domain, the spacer domain is selected from a hinge domain and/or CH2 and CH3 regions of an immunoglobulin (e.g., IgG1 or IgG4); preferably, the hinge domain comprises a hinge region of CD8α, IgG4, PD1, CD152 or CD154; more preferably, the hinge domain comprises a hinge region of CD8α or IgG4;
preferably, the transmembrane domain is selected from one or more transmembrane regions selected from the group consisting of: α, β or ζ chain of T cell receptor, CD3ε, CD3ζ, CD4, CD5, CD8α, CD28, CD137, CD152, CD154 and PD1; preferably, the transmembrane domain is selected from one or more transmembrane regions selected from the group consisting of: CD8α, CD28, CD4, PD1, CD152 and CD154; preferably, the transmembrane domain comprises the transmembrane region of CD8α or CD28;
preferably, the intracellular signaling domain comprises a primary signaling domain and/or a costimulatory signaling domain; preferably, the intracellular signaling domain comprises a primary signaling domain and at least one costimulatory signaling domain; preferably, the primary signaling domain comprises an immunoreceptor tyrosine activation motif (ITAM); preferably, the primary signaling domain comprises an intracellular signaling domain of a protein selected from the group consisting of: CD3ζ, FcRγ, FcRβ, CD3γ, CD3δ, CD3ε, CDS, CD22, CD79a, CD79b and CD66d; more preferably, the primary signaling domain comprises an intracellular signaling domain of CD3ζ; preferably, the costimulatory signaling domain comprises an intracellular signaling domain of a protein selected from the group consisting of: CARD11, CD2, CD7, CD27, CD28, CD30, CD134 (OX40), CD137 (4-1BB), CD150 (SLAMF1), CD270 (HVEM), CD278 (ICOS) and DAP10; more preferably, the costimulatory signaling domain is selected from an intracellular signaling domain of CD28 or an intracellular signaling domain of CD137 (4-1BB) or a combination thereof;
preferably, the first CAR and the second CAR further comprise a signal peptide at their N-terminal; preferably, the signal peptide comprises a heavy chain signal peptide (e.g., heavy chain signal peptide of IgG1), a granulocyte-macrophage colony stimulating factor receptor 2 (GM-CSFR2) signal peptide, or a CD8α signal peptide; more preferably, the signal peptide is selected from a CD8α signal peptide;
preferably, the first CAR has an amino acid sequence selected from the group consisting of: (1) an amino acid sequence as set forth in SEQ ID NO: 29; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 29;
preferably, the second CAR has an amino acid sequence selected from the group consisting of: (1) an amino acid sequence as set forth in SEQ ID NO: 30; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 30;
more preferably, the CAR construct has an amino acid sequence selected from the group consisting of: (1) an amino acid sequence as set forth in SEQ ID NO:51; (2) a sequence having a sequence identity of at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence as set forth in SEQ ID NO: 51.

18. An isolated nucleic acid molecule, which comprises a nucleotide sequence encoding the chimeric antigen receptor CAR according to claim 6, preferably, the isolated nucleic acid molecule comprising a nucleotide sequence selected from the group consisting of: (1) a nucleotide sequence as set forth in any one of SEQ ID NOs: 43-48, 65, 67; (2) a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the nucleotide sequence as set forth in SEQ ID NO: 43-48, 65, 67.

19. A nucleic acid construct, which comprises a first nucleotide sequence and a second nucleotide sequence encoding the first CAR and the second CAR in the CAR construct according to claim 17 respectively;
preferably, the first nucleotide sequence and second nucleotide sequence are linked in any order by a nucleotide sequence encoding a self-cleaving peptide (e.g., P2A, E2A, F2A or T2A); preferably, the self-cleaving peptide is P2A (e.g., P2A of the sequence as set forth in SEQ ID NO: 50);
preferably, the nucleic acid construct comprises a nucleotide sequence selected from the group consisting of: (1) a nucleotide sequence as set forth in SEQ ID NO: 52; (2) a sequence having a sequence identity of at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% as compared to the nucleotide sequence as set forth in SEQ ID NO: 52.

20. A vector, which comprises an isolated nucleic acid molecule comprising a nucleotide sequence encoding the CAR of claim 6;
preferably, the vector is selected from the group consisting of DNA vector, RNA vector, plasmid, transposon vector, CRISPR/Cas9 vector or viral vector;
preferably, the vector is an expression vector;
preferably, the vector is an episomal vector;
preferably, the vector is a viral vector; more preferably, the viral vector is a lentiviral vector, an adenoviral vector or a retroviral vector.

21. A host cell, which comprises (i) an isolated nucleic acid molecule comprising a nucleotide sequence encoding the CAR of claim 6 or (ii) a vector comprising the isolated nucleic acid molecule;
preferably, the host cell is selected from an immune cell (e.g., human immune cell); more preferably, the immune cell is selected from the group consisting of T lymphocyte, NK cell, monocyte, macrophage or dendritic cell and any combination thereof.

22. A method for preparing a cell expressing a chimeric antigen receptor, comprising: (1) providing a host cell; (2) obtaining a host cell capable of expressing the chimeric antigen receptor; wherein step (2) comprises introducing (i) an isolated nucleic acid molecule comprising a nucleotide sequence encoding the CAR of claim 6 or (ii) a vector comprising the isolated nucleic acid molecule into the host cell of step (1);
preferably, the host cell is selected from an immune cell (e.g., human immune cell); preferably, the immune cell is selected from the group consisting of T lymphocyte, NK cell, monocyte, macrophage or dendritic cell and any combination thereof;
preferably, in step (1), the immune cell undergoes pretreatment, and the pretreatment comprises sorting, activation and/or proliferation of the immune cell; more preferably, the pretreatment comprises contacting the immune cell with an anti-CD3 antibody and an anti-CD28 antibody, thereby stimulating the immune cell and inducing its proliferation, thereby generating a pretreated immune cell;
preferably, in step (2), the nucleic acid molecule or the vector is introduced into the host cell by viral infection;
preferably, in step (2), the nucleic acid molecule or the vector is introduced into the host cell by means of non-viral vector transfection, such as calcium phosphate transfection, DEAE-dextran-mediated transfection, microinjection, transposon vector system, CRISPR/Cas9 vector, TALEN method, ZFN method or electroporation method;
preferably, a step of expanding the host cell obtained in the step (2) is further comprised after step (2).

23. An engineered immune cell, which expresses the chimeric antigen receptor targeting BCMA and CD19 of claim 6;
optionally, the chimeric antigen receptor targeting BCMA and CD19 is expressed on the surface of the engineered immune cell;
optionally, the engineered immune cell also expresses a CAR that is not specific for BCMA and CD19; preferably, the CAR that is not specific for BCMA and CD19 has specificity for a target selected from the group consisting of: CD20, CD22, CD33, CD123 and CD138;
optionally, the immune cell further comprises knockout of one or more endogenous genes, wherein the endogenous genes encode TCRα, TCRβ, CD52, glucocorticoid receptor (GR), deoxycytidine kinase (dCK), or immune checkpoint protein such as PD-1;
preferably, the immune cell is derived from T lymphocyte, NK cell, monocyte, macrophage or dendritic cell and any combination thereof; and/or the immune cell is obtained from a patient or a healthy donor.

24. (canceled)

25. An immune cell composition, comprising the engineered immune cell according to claim 23; optionally, the composition also comprises an unmodified and/or unsuccessfully engineered immune cell; preferably, the number of engineered immune cells accounts for 10% to 100%, more preferably 40% to 80%, of the total number of cells in the immune cell composition.

26. A kit, wherein the kit comprises: (i) the bispecific antibody or antigen-binding fragment thereof according to claim 1, or (ii) a chimeric antigen receptor (CAR) comprising the bispecific antibody or antigen-binding fragment thereof, or (iii) a nucleic acid molecule comprising a nucleotide sequence encoding the CAR, or (iv) a vector comprising the nucleic acid molecule, or (v) a host cell comprising the nucleic acid molecule or vector;
optionally, the kit is used to prepare a chimeric antigen receptor or CAR construct targeting BCMA and CD19, or to prepare a cell expressing the chimeric antigen receptor or CAR construct.

27. (canceled)

28. A pharmaceutical composition, which comprises a pharmaceutically acceptable carrier and/or excipient and one of the following: (i) the bispecific antibody or antigen-binding fragment thereof of claim 1, or (ii) a chimeric antigen receptor (CAR) comprising the bispecific antibody or antigen-binding fragment thereof, or (iii) an isolated nucleic acid molecule comprising a nucleotide sequence encoding the CAR, or (iv) a vector comprising the isolated nucleic acid molecule, or (v) a host cell comprising the isolated nucleic acid molecule or vector, or (vi) an engineered immune cell expressing the CAR, or (vii) an immune cell composition comprising the engineered immune cell;
preferably, the pharmaceutical composition further comprises an additional pharmaceutically active agent; more preferably, the additional pharmaceutically active agent is selected from an additional antibody, a fusion protein or a drug (e.g., an anti-tumor drug, such as a drug used in radiotherapy or a chemotherapy drug).

29. (canceled)

30. A method for preventing and/or treating a B cell-related disease or condition in a subject (such as a human), the method comprising administering to a subject in need thereof an effective amount of an active agent selected from the following: (i) the bispecific antibody or antigen-binding fragment thereof according to claim 1, or (ii) a chimeric antigen receptor (CAR) comprising the bispecific antibody or antigen-binding fragment thereof, or (iii) an isolated nucleic acid molecule comprising a nucleotide sequence encoding the CAR, or (iv) a vector comprising the isolated nucleic acid molecule, or (v) a host cell comprising the isolated nucleic acid molecule or vector, or (vi) an engineered immune cell expressing the CAR, or (vii) an immune cell composition comprising the engineered immune cell, or (viii) a pharmaceutical composition comprising any one of (i)-(vii);
preferably, the B cell-related disease or condition is selected from the group consisting of multiple myeloma, non-Hodgkin's lymphoma, B cell proliferation of uncertain malignant potential, lymphomatoid granulomatosis, post-transplant lymphoproliferative disorder, immunomodulatory disorder, rheumatic arthritis, myasthenia gravis, idiopathic thrombocytopenic purpura, antiphospholipid syndrome, Chagas' disease, Graves' disease, Wegener's granulomatosis, polyarteritis nodosa, Sjogren's syndrome, pemphigus vulgaris, scleroderma, multiple sclerosis, antiphospholipid syndrome, ANCA-associated vasculitis, Goodpasture's disease, Kawasaki disease, autoimmune hemolytic anemia and rapidly progressive glomerulonephritis, heavy chain disease, primary or immunocyte-associated amyloidosis or monoclonal gammopathy of undetermined significance, systemic lupus erythematosus;
preferably, the B cell-related disease or condition is a B cell malignancy, such as multiple myeloma (MM) or non-Hodgkin's lymphoma (NHL);
preferably, the B cell-related disease or condition is a B cell- and plasma cell-related disease or condition, such as an autoimmune disease such as systemic lupus erythematosus;
preferably, the active agent is administered in combination with one or more of the following:
(i) an agent that increases the efficacy of a cell comprising a CAR nucleic acid or CAR polypeptide;
(ii) an agent that ameliorates one or more side effects associated with administration of a cell comprising a CAR nucleic acid or CAR polypeptide;
(iii) an additional agent for treating a disease associated with BCMA and CD19;
(iv) a second therapy, which is selected from the group consisting of surgery, chemotherapy, radiotherapy, immunotherapy, gene therapy, DNA therapy, RNA therapy, nanotherapy, viral therapy, adjuvant therapy, and any combination thereof.

31. A method for preventing and/or treating a B cell-related disease or condition in a subject (e.g., a human), the method comprising the following steps: (1) providing an immune cell required by the subject; (2) introducing the isolated nucleic acid molecule according to claim 18 into the immune cell of step (1) to obtain an immune cell expressing a chimeric antigen receptor; (3) administering the immune cell obtained in step (2) to the subject;
optionally, in step (3), the total dose of the immune cells comprises 1 to 5×10⁷ or 1 to 5×10⁸ cells;
preferably, in step (3), the total dose of the immune cells is administered to the subject in divided doses.

32. The chimeric antigen receptor (CAR) according to claim 6, characterized by one or more of the following:
(i) the transmembrane domain is selected from one or more transmembrane regions selected from the group consisting of: α, β or λ chain of T cell receptor, CD3ε, CD3ζ, CD4, CD5, CD8α, CD28, CD137, CD152, CD154 and PD1; preferably, the transmembrane domain is selected from one or more transmembrane regions selected from the group consisting of: CD8α, CD28, CD4, PD1, CD152 and CD154; preferably, the transmembrane domain comprises the transmembrane region of CD8α or CD28;
(ii) the spacer domain is located between the antigen-binding domain and the transmembrane domain, the spacer domain is selected from a hinge domain and/or CH2 and CH3 regions of an immunoglobulin (e.g., IgG1 or IgG4); preferably, the hinge domain comprises a hinge region of CD8α, IgG4, PD1, CD152 or CD154; more preferably, the hinge domain comprises a hinge region of CD8α or IgG4;
(iii) the chimeric antigen receptor (CAR) further comprises a signal peptide at its N-terminal; preferably, the signal peptide comprises a heavy chain signal peptide (e.g., heavy chain signal peptide of IgG1), a granulocyte-macrophage colony stimulating factor receptor 2 (GM-CSFR2) signal peptide, or a CD8α signal peptide; more preferably, the signal peptide is selected from a CD8α signal peptide;
(iv) the intracellular signaling domain comprises a primary signaling domain and/or a costimulatory signaling domain; preferably, the intracellular signaling domain comprises a primary signaling domain and at least one costimulatory signaling domain; preferably, the primary signaling domain comprises an immunoreceptor tyrosine activation motif (ITAM); preferably, the primary signaling domain comprises an intracellular signaling domain of a protein selected from the group consisting of: CD3ζ, FcRγ, FcRβ, CD3γ, CD3δ, CD3ε, CDS, CD22, CD79a, CD79b and CD66d; more preferably, the primary signaling domain comprises an intracellular signaling domain of CD3ζ; preferably, the costimulatory signaling domain comprises an intracellular signaling domain of a protein selected from the group consisting of: CARD11, CD2, CD7, CD27, CD28, CD30, CD134 (OX40), CD137 (4-1BB), CD150 (SLAMF1), CD270 (HVEM), CD278 (ICOS) and DAP10; more preferably, the costimulatory signaling domain is selected from an intracellular signaling domain of CD28 or an intracellular signaling domain of CD137 (4-1BB) or a combination thereof.

33. A vector, which comprises a nucleic acid construct comprising a first nucleotide sequence and a second nucleotide sequence encoding the first CAR and the second CAR in the CAR construct of claim 17 respectively;
preferably, the vector is selected from the group consisting of DNA vector, RNA vector, plasmid, transposon vector, CRISPR/Cas9 vector or viral vector;
preferably, the vector is an expression vector;
preferably, the vector is an episomal vector;
preferably, the vector is a viral vector; more preferably, the viral vector is a lentiviral vector, an adenoviral vector or a retroviral vector.

34. A host cell, which comprises: (i) a nucleic acid construct comprising a first nucleotide sequence and a second nucleotide sequence encoding the first CAR and the second CAR in the CAR construct of claim 17 respectively; or (ii) a vector comprising the nucleic acid construct;
preferably, the host cell is selected from an immune cell (e.g., human immune cell); more preferably, the immune cell is selected from the group consisting of T lymphocyte, NK cell, monocyte, macrophage or dendritic cell and any combination thereof.

35. A method for preparing a cell expressing a CAR construct, comprising: (1) providing a host cell; (2) obtaining a host cell capable of expressing the CAR construct; wherein step (2) comprises introducing (i) a nucleic acid construct comprising a first nucleotide sequence and a second nucleotide sequence encoding the first CAR and the second CAR in the CAR construct of claim 17 respectively; or (ii) a vector comprising the nucleic acid construct into the host cell of step (1);
preferably, the host cell is selected from an immune cell (e.g., human immune cell); preferably, the immune cell is selected from the group consisting of T lymphocyte, NK cell, monocyte, macrophage or dendritic cell and any combination thereof;
preferably, in step (1), the immune cell undergoes pretreatment, and the pretreatment comprises sorting, activation and/or proliferation of the immune cell; more preferably, the pretreatment comprises contacting the immune cell with an anti-CD3 antibody and an anti-CD28 antibody, thereby stimulating the immune cell and inducing its proliferation, thereby generating a pretreated immune cell;
preferably, in step (2), the nucleic acid molecule or the vector is introduced into the host cell by viral infection;
preferably, in step (2), the nucleic acid molecule or the vector is introduced into the host cell by means of non-viral vector transfection, such as calcium phosphate transfection, DEAE-dextran-mediated transfection, microinjection, transposon vector system, CRISPR/Cas9 vector, TALEN method, ZFN method or electroporation method;
preferably, a step of expanding the host cell obtained in the step (2) is further comprised after step (2).

36. An engineered immune cell, which expresses the chimeric antigen receptor (CAR) construct of claim 17;
optionally, the CAR construct targeting BCMA and CD19 is expressed on the surface of the engineered immune cell in the form of co-expression of independent BCMA-targeting chimeric antigen receptor and CD19-targeting chimeric antigen receptor;
optionally, the engineered immune cell also expresses a CAR that is not specific for BCMA and CD19; preferably, the CAR that is not specific for BCMA and CD19 has specificity for a target selected from the group consisting of: CD20, CD22, CD33, CD123 and CD138;
optionally, the immune cell further comprises knockout of one or more endogenous genes, wherein the endogenous genes encode TCRα, TCRβ, CD52, glucocorticoid receptor (GR), deoxycytidine kinase (dCK), or immune checkpoint protein such as PD-1;
preferably, the immune cell is derived from T lymphocyte, NK cell, monocyte, macrophage or dendritic cell and any combination thereof; and/or the immune cell is obtained from a patient or a healthy donor.

37. An immune cell composition, comprising the engineered immune cell according to claim 35; optionally, the composition also comprises an unmodified and/or unsuccessfully engineered immune cell; preferably, the number of engineered immune cells accounts for 10% to 100%, more preferably 40% to 80%, of the total number of cells in the immune cell composition.

38. A kit, wherein the kit comprises (i) the CAR construct of claim 17, or (ii) a nucleic acid construct comprising a first nucleotide sequence and a second nucleotide sequence encoding the first CAR and the second CAR in the CAR construct respectively, or (iii) a vector comprising the nucleic acid construct, or (v) a host cell comprising the nucleic acid construct or vector;
optionally, the kit is used to prepare a CAR construct targeting BCMA and CD19, or to prepare a cell expressing CAR construct.

39. A pharmaceutical composition, which comprises a pharmaceutically acceptable carrier and/or excipient and one of the following: (i) the CAR construct of claim 17, or (ii) a nucleic acid construct comprising a first nucleotide sequence and a second nucleotide sequence encoding the first CAR and the second CAR in the CAR construct respectively, or (iii) a vector comprising the nucleic acid construct, or (iv) a host cell comprising the nucleic acid construct or vector, or (v) an engineered immune cell expressing the CAR construct, or (vi) an immune cell composition comprising the engineered immune cell;
preferably, the pharmaceutical composition further comprises an additional pharmaceutically active agent; more preferably, the additional pharmaceutically active agent is selected from an additional antibody, a fusion protein or a drug (e.g., an anti-tumor drug, such as a drug used in radiotherapy or a chemotherapy drug).

40. A method for preventing and/or treating a B cell-related disease or condition in a subject (such as a human), the method comprising administering to a subject in need thereof an effective amount of an active agent selected from the following: (i) the CAR construct of claim 17, or (ii) a nucleic acid construct comprising a first nucleotide sequence and a second nucleotide sequence encoding the first CAR and the second CAR in the CAR construct respectively, or (iii) a vector comprising the nucleic acid construct, or (iv) a host cell comprising the nucleic acid construct or vector, or (v) an engineered immune cell expressing the CAR construct, or (vi) an immune cell composition comprising the engineered immune cell, or (vii) a pharmaceutical composition comprising any one of (i)-(vi);
preferably, the B cell-related disease or condition is selected from the group consisting of multiple myeloma, non-Hodgkin's lymphoma, B cell proliferation of uncertain malignant potential, lymphomatoid granulomatosis, post-transplant lymphoproliferative disorder, immunomodulatory disorder, rheumatic arthritis, myasthenia gravis, idiopathic thrombocytopenic purpura, antiphospholipid syndrome, Chagas' disease, Graves' disease, Wegener's granulomatosis, polyarteritis nodosa, Sjogren's syndrome, pemphigus vulgaris, scleroderma, multiple sclerosis, antiphospholipid syndrome, ANCA-associated vasculitis, Goodpasture's disease, Kawasaki disease, autoimmune hemolytic anemia and rapidly progressive glomerulonephritis, heavy chain disease, primary or immunocyte-associated amyloidosis or monoclonal gammopathy of undetermined significance, systemic lupus erythematosus;
preferably, the B cell-related disease or condition is a B cell malignancy, such as multiple myeloma (MM) or non-Hodgkin's lymphoma (NHL);
preferably, the B cell-related disease or condition is a B cell- and plasma cell-related disease or condition, such as an autoimmune disease such as systemic lupus erythematosus;
preferably, the active agent is administered in combination with one or more of the following:
(i) an agent that increases the efficacy of a cell comprising a CAR nucleic acid or CAR polypeptide;
(ii) an agent that ameliorates one or more side effects associated with administration of a cell comprising a CAR nucleic acid or CAR polypeptide;
(iii) an additional agent for treating a disease associated with BCMA and CD19;
(iv) a second therapy, which is selected from the group consisting of surgery, chemotherapy, radiotherapy, immunotherapy, gene therapy, DNA therapy, RNA therapy, nanotherapy, viral therapy, adjuvant therapy, and any combination thereof.

41. A method for preventing and/or treating a B cell-related disease or condition in a subject (e.g., a human), the method comprising the following steps: (1) providing an immune cell required by the subject; (2) introducing the nucleic acid construct of claim 19 into the immune cell of step (1) to obtain an immune cell expressing a CAR construct; (3) administering the immune cell obtained in step (2) to the subject;
optionally, in step (3), the total dose of the immune cells comprises 1 to 5×10⁷ or 1 to 5×10⁸ cells;
preferably, in step (3), the total dose of the immune cells is administered to the subject in divided doses.


SEQUENCE LISTING (81 sequences)

SEQ ID NO: 1 - protein, 119 aa, Artificial sequence
  H-BCMA VH Humanized antibody BCMA heavy chain variable region
QVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVS

SEQ ID NO: 2 - protein, 113 aa, Artificial sequence
  H-BCMA VL Humanized antibody BCMA light chain variable region
DIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRA

SEQ ID NO: 3 - protein, 120 aa, Artificial sequence
  H-CD19 VH Humanized antibody CD19 heavy chain variable region
QVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSS

SEQ ID NO: 4 - protein, 106 aa, Artificial sequence
  H-CD19 VL Humanized antibody CD19 light chain variable region
EIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEI

SEQ ID NO: 5 - protein, 7 aa, Artificial sequence
  H-BCMA VH CDR1
GYTFTDY

SEQ ID NO: 6 - protein, 6 aa, Artificial sequence
  H-BCMA VH CDR2
NTETGE

SEQ ID NO: 7 - protein, 11 aa, Artificial sequence
  H-BCMA VH CDR3
SLYYGYSWFTY

SEQ ID NO: 8 - protein, 16 aa, Artificial sequence
  H-BCMA VL CDR1
RSSQSLVHSNGNTFLH

SEQ ID NO: 9 - protein, 7 aa, Artificial sequence
  H-BCMA VL CDR2
KVSNRFS

SEQ ID NO: 10 - protein, 8 aa, Artificial sequence
  H-BCMA VL CDR3
MQSTHVLT

SEQ ID NO: 11 - protein, 8 aa, Artificial sequence
  H-CD19 VH CDR1
GVSLPDYG

SEQ ID NO: 12 - protein, 10 aa, Artificial sequence
  H-CD19 VH CDR2
IWGSETTYYN

SEQ ID NO: 13 - protein, 14 aa, Artificial sequence
  H-CD19 VH CDR3
AKHYYYGGSYAMDY

SEQ ID NO: 14 - protein, 6 aa, Artificial sequence
  H-CD19 VL CDR1
QDISKY

SEQ ID NO: 15 - protein, 11 aa, Artificial sequence
  H-CD19 VL CDR2
HTSRLHSGIPD

SEQ ID NO: 16 - protein, 9 aa, Artificial sequence
  H-CD19 VL CDR3
QQGNTLPYT

SEQ ID NO: 17 - protein, 5 aa, Artificial sequence
  Linker1 amino acid sequence
GGGGS

SEQ ID NO: 18 - protein, 15 aa, Artificial sequence
  Linker2 amino acid sequence
GGGGSGGGGSGGGGS

SEQ ID NO: 19 - protein, 20 aa, Artificial sequence
  Linker3 amino acid sequence
GGGGSGGGGSGGGGSGGGGS

SEQ ID NO: 20 - protein, 15 aa, Artificial sequence
  Linker4 amino acid sequence
EAAAKEAAAKEAAAK

SEQ ID NO: 21 - protein, 45 aa, Artificial sequence
  CD8(alpha) hinge region amino acid sequence
TTTPAPRPPTPAPTIASQPLSLRPEACRPAAGGAVHTRGLDFACD

SEQ ID NO: 22 - protein, 24 aa, Artificial sequence
  CD8TM amino acid sequence
IYIWAPLAGTCGVLLLSLVITLYC

SEQ ID NO: 23 - protein, 42 aa, Artificial sequence
  4-1BB intracellular signaling domain amino acid sequence
KRGRKKLLYIFKQPFMRPVQTTQEEDGCSCRFPEEEEGGCEL

SEQ ID NO: 24 - protein, 112 aa, Artificial sequence
  CD3(Zeta) intracellular signaling domain amino acid sequence-1
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 246 aa, Artificial sequence
  Anti-murine-BCMA scFv amino acid sequence
DVVMTQSPLSLPVTLGQPASISCRSSQSLVHSNGNTFLHWYQQRPGQSPRLLIFKVSNRFSGVPDRFSGSGSGTDFTLKISRVEAEDVGVYFCSQSTHVLTFGQGTKLEIKGGGGSGGGGSGGGGSQIQLVQSGAEVKKPGASVKVSCKASGYTFTDYSMDWLRQAPGQGLEWMGWINTETGESTYAQKLQGRVTFSLDTSTSTAYMELRSLRSDDTAVYFCAPSLFYGYSWFPYWGQGTLVTVSS

SEQ ID NO: 26 - protein, 242 aa, Artificial sequence
  FMC63 (murine-CD19 antibody) scFv amino acid sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 27 - protein, 247 aa, Artificial sequence
  H-BCMA scFv amino acid sequence
DIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVS

SEQ ID NO: 28 - protein, 241 aa, Artificial sequence
  H-CD19 scFv amino acid sequence
QVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSGGGGSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEI

SEQ ID NO: 29 - protein, 491 aa, Artificial sequence
  H-BCMA CAR amino acid sequence
MALPVTALLLPLALLLHAARPDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 485 aa, Artificial sequence
  H-CD19 CAR amino acid sequence
MALPVTALLLPLALLLHAARPQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSGGGGSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 738 bp, Artificial sequence
  Murine-BCMA scFv encoding nucleotide sequence
gacgtggtcatgacacagagccctctgagcctgcctgtgacactgggacagcctgccagcatcagctgtagaagcagccagagcctggtgcacagcaacggcaataccttcctgcactggtatcagcagaggcccggacagtctcccagactgctgatcttcaaggtgtccaaccggttcagcggcgtgcccgatagattttctggcagcggctctggcaccgacttcaccctgaagatctccagagtggaagccgaggacgtgggcgtgtacttctgtagccagagcacccacgtgctgacctttggccagggcaccaagctggaaatcaagggaggcggaggttctggcggcggaggaagtggtggcggaggctctcagattcagctggtgcagtctggcgccgaagtgaagaaacctggcgcctctgtgaaggtgtcctgcaaggccagcggctacacctttaccgactacagcatggactggctgagacaggcccctggacaaggcttggaatggatgggctggatcaacaccgagacaggcgagagcacatacgcccagaaactgcagggcagagtgaccttcagcctggacaccagcacaagcaccgcctacatggaactgcggagcctgagatccgatgataccgccgtgtacttctgcgcccctagcctgttttacggctacagctggttcccctattggggccagggaaccctggtcacagtcagctct

SEQ ID NO: 32 - DNA, 726 bp, Artificial sequence
  FMC63 (murine-CD19 antibody) scFv encoding nucleotide sequence
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 33 - DNA, 741 bp, Artificial sequence
  H-BCMA scFv encoding nucleotide sequence
gacatcgtgatgacacagagccctctgagcctgcctgtgacacctggcgaacctgccagcatcagctgtagaagcagccagagcctggtgcacagcaacggcaataccttcctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgtatgcagagcacccacgtgctgacctttggcggcggaacaaagctggaaatcaagcgggctggtggaggtggcagcggaggaggtgggtccggcggtggaggaagccaggttcagctggttcagtctggcgccgaagtgaagaaacctggcgcctctgtgaaggtgtcctgcaaggccagcggctacacctttaccgactacctgatgcactgggtccgacaggctccaggacaaggcttggaatggatgggctggatcaacaccgagacaggcgagcctacctacgccgacgatttcaagggcagagtgaccatgaccagagacaccagcacctccaccgtgtacatggaactgagcagcctgagaagcgaggacaccgccgtgtactactgtgcccctagcctgtactacggctactcctggtttacctactggggccagggcaccctggtcacagtttct

SEQ ID NO: 34 - DNA, 723 bp, Artificial sequence
  H-CD19 scFv encoding nucleotide sequence
caggtcaccctgagagagagcggaccagccctggtgaaacctacacagaccctgaccctgacctgcacctttagcggagtgtccctgccagactacggagtgtcttggatccggcagccaccaggaaaagctctcgagtggctcgcagtgatttggggcagcgagaccacctactacaacagcgccctgaagagcaggctgaccatcagcaaggacaccagcaagaaccaggtggtgctgaccatgaccaacatggaccccgtggacaccgccacctactattgcgccaagcactactactacggcggcagctacgccatggactattggggacagggcaccaccgtgacagtgtctagcggaggaggaggaagcggaggaggaggatctggaggaggaggcagcgagattgtgctgacacagagcccagccacactgtctctgagcccaggagagagagccacactgtcttgcagggccagccaggacatcagcaagtacctgaattggtaccagcagaagcccggccaggctcctaggctgctgatctaccacaccagcaggctgcacagcggcatcccagacagattcagcggaagcggcagcggcacagacttcaccctgaccatcagcagactggagcccgaggacatcgccgtgtactattgccagcagggcaacaccctgccctacacctttggacagggcaccaagctggagatc

SEQ ID NO: 35 - DNA, 1473 bp, Artificial sequence
  H-BCMA CAR encoding nucleic acid sequence
atggccctccctgtcaccgccctgctgcttccgctggctcttctgctccacgccgctcggcccgacatcgtgatgacacagagccctctgagcctgcctgtgacacctggcgaacctgccagcatcagctgtagaagcagccagagcctggtgcacagcaacggcaataccttcctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgtatgcagagcacccacgtgctgacctttggcggcggaacaaagctggaaatcaagcgggctggtggaggtggcagcggaggaggtgggtccggcggtggaggaagccaggttcagctggttcagtctggcgccgaagtgaagaaacctggcgcctctgtgaaggtgtcctgcaaggccagcggctacacctttaccgactacctgatgcactgggtccgacaggctccaggacaaggcttggaatggatgggctggatcaacaccgagacaggcgagcctacctacgccgacgatttcaagggcagagtgaccatgaccagagacaccagcacctccaccgtgtacatggaactgagcagcctgagaagcgaggacaccgccgtgtactactgtgcccctagcctgtactacggctactcctggtttacctactggggccagggcaccctggtcacagtttctaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 36 - DNA, 1455 bp, Artificial sequence
  H-CD19 CAR encoding nucleic acid sequence
atggccctccctgtcaccgccctgctgcttccgctggctcttctgctccacgccgctcggccccaggtcaccctgagagagagcggaccagccctggtgaaacctacacagaccctgaccctgacctgcacctttagcggagtgtccctgccagactacggagtgtcttggatccggcagccaccaggaaaagctctcgagtggctcgcagtgatttggggcagcgagaccacctactacaacagcgccctgaagagcaggctgaccatcagcaaggacaccagcaagaaccaggtggtgctgaccatgaccaacatggaccccgtggacaccgccacctactattgcgccaagcactactactacggcggcagctacgccatggactattggggacagggcaccaccgtgacagtgtctagcggaggaggaggaagcggaggaggaggatctggaggaggaggcagcgagattgtgctgacacagagcccagccacactgtctctgagcccaggagagagagccacactgtcttgcagggccagccaggacatcagcaagtacctgaattggtaccagcagaagcccggccaggctcctaggctgctgatctaccacaccagcaggctgcacagcggcatcccagacagattcagcggaagcggcagcggcacagacttcaccctgaccatcagcagactggagcccgaggacatcgccgtgtactattgccagcagggcaacaccctgccctacacctttggacagggcaccaagctggagatcaccactaccccagcaccgaggccacccaccccggctcctaccatcgcctcccagcctctgtccctgcgtccggaggcatgtagacccgcagctggtggggccgtgcatacccgtggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 37 - protein, 752 aa, Artificial sequence
  TanCAR 01 full-length amino acid sequence
MALPVTALLLPLALLLHAARPDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSGGGGSGGGGSGGGGSGGGGSQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSGGGGSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 747 aa, Artificial sequence
  TanCAR 02 full-length amino acid sequence
MALPVTALLLPLALLLHAARPDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSEAAAKEAAAKEAAAKQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSGGGGSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 752 aa, Artificial sequence
  TanCAR 03 full-length amino acid sequence
MALPVTALLLPLALLLHAARPQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSGGGGSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEIGGGGSGGGGSGGGGSGGGGSDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 747 aa, Artificial sequence
  TanCAR 04 full-length amino acid sequence
MALPVTALLLPLALLLHAARPQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSGGGGSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEIEAAAKEAAAKEAAAKDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 732 aa, Artificial sequence
  TanCAR 05 full-length amino acid sequence
MALPVTALLLPLALLLHAARPDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSGGGGSGGGGSGGGGSGGGGSQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 732 aa, Artificial sequence
  TanCAR 06 full-length amino acid sequence
MALPVTALLLPLALLLHAARPQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 2256 bp, Artificial sequence
  TanCAR 01 encoding nucleotide sequence
atggccctgccagtgaccgccctgctgctgccactggccctgctgctgcacgccgccagacccgacatcgtgatgacccagagcccactgtccctgccagtgacaccaggagagcctgcaagcatctcctgcaggagctcccagtccctggtgcactctaacggcaataccttcctgcactggtatctgcagaagccaggccagtccccccagctgctgatctacaaggtgagcaaccggttctccggcgtgccagacagattttctggcagcggctccggcaccgatttcacactgaagatcagcagagtggaggccgaggacgtgggcgtgtactattgcatgcagtccacccacgtgctgacatttggcggcggcaccaagctggagatcaagagggccggaggaggaggatctggaggaggaggctccggcggcggcggctcccaggtgcagctggtgcagagcggagcagaggtgaagaagccaggagcctctgtgaaggtgagctgtaaggcctccggctataccttcacagattacctgatgcactgggtgcgccaggcacctggacagggactggagtggatgggctggatcaataccgagacaggcgagccaacctatgccgacgacttcaagggccgggtgaccatgacaagagacacctctacaagcaccgtgtacatggagctgtctagcctgcggtccgaggataccgccgtgtactattgcgccccttccctgtactatggctattcttggttcacatactggggccagggcaccctggtgacagtgtctggaggaggaggaagtggaggaggaggatccggcggcggcggctctggcggcggcggcagccaggtgaccctgagggagagcggccccgccctggtgaagcctacacagaccctgacactgacctgtacattttctggcgtgagcctgcccgattacggcgtgtcttggatcaggcagccacctggcaaggccctggagtggctggccgtgatctggggcagcgagaccacatactataactctgccctgaagagccggctgaccatctccaaggacacatctaagaaccaggtggtgctgaccatgacaaatatggaccctgtggataccgccacatactattgcgccaagcactactattacggcggctcctatgccatggattattggggacagggcactacagtgaccgtgtcctctggaggaggaggatccggaggaggaggaagcggcggcggcggcagcgagatcgtgctgacccagagccctgccacactgtccctgtctccaggagagagggccacactgagctgtagggcatcccaggacatctctaagtatctgaactggtaccagcagaagcctggccaggccccaaggctgctgatctatcacaccagcaggctgcactccggcatccccgaccggttcagcggatccggatctggcacagacttcaccctgacaatcagccggctggagccagaggatatcgccgtgtattactgtcagcagggcaataccctgccctacacatttggccagggaactaagctggagatcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 44 - DNA, 2241 bp, Artificial sequence
  TanCAR 02 encoding nucleotide sequence
atggccctgccagtgaccgccctgctgctgccactggccctgctgctgcacgccgccagacccgacatcgtgatgacccagagcccactgtccctgccagtgacaccaggagagcctgcaagcatctcctgcaggagctcccagtccctggtgcactctaacggcaataccttcctgcactggtatctgcagaagccaggccagagcccccagctgctgatctacaaggtgagcaaccggttctccggcgtgccagacagattttctggcagcggctccggcaccgatttcacactgaagatctccagagtggaggccgaggacgtgggcgtgtactattgcatgcagtctacccacgtgctgacatttggcggcggcaccaagctggagatcaagagggccggaggaggaggatctggaggaggaggctccggcggcggcggctcccaggtgcagctggtgcagagcggagcagaggtgaagaagccaggagcctctgtgaaggtgagctgtaaggcctccggctataccttcacagattacctgatgcactgggtgcgccaggcacctggacagggactggagtggatgggctggatcaataccgagacaggcgagccaacctatgccgacgacttcaagggccgggtgaccatgacaagagacacctctacaagcaccgtgtacatggagctgtctagcctgcggtccgaggataccgccgtgtactattgcgccccttctctgtactatggctatagctggttcacatactggggccagggcaccctggtgacagtgtctgaggcagcagcaaaggaggcagcagccaaggaggcagcagcaaagcaggtgaccctgagggagtccggacccgccctggtgaagcctacacagaccctgacactgacctgtacattttctggcgtgagcctgcccgattacggcgtgagctggatcaggcagccacctggcaaggccctggagtggctggccgtgatctggggctccgagaccacatactataactctgccctgaagagccggctgaccatctccaaggacacatctaagaaccaggtggtgctgaccatgacaaatatggaccctgtggataccgccacatactattgcgccaagcactactattacggcggcagctatgccatggattattggggacagggcactacagtgaccgtgtcctctggcggcggcggctctggaggaggaggatccggaggaggaggatccgagatcgtgctgacccagtcccctgccacactgtccctgtctccaggagagagggccacactgagctgtagggcatcccaggacatctctaagtatctgaactggtaccagcagaagcctggccaggccccaaggctgctgatctatcacaccagcaggctgcactccggcatccccgaccggttcagcggatccggatctggcacagacttcaccctgacaatcagccggctggagccagaggatatcgccgtgtattactgtcagcagggcaataccctgccctacacatttggccagggaactaagctggagatcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 45 - DNA, 2256 bp, Artificial sequence
  TanCAR 03 encoding nucleotide sequence
atggccctgccagtgaccgccctgctgctgccactggccctgctgctgcacgcagcaaggcctcaggtgacactgagggagagcggacccgccctggtgaagcctacccagacactgaccctgacatgcaccttcagcggcgtgtccctgccagactacggcgtgtcctggatcaggcagccacctggcaaggccctggagtggctggccgtgatctggggctctgagaccacatactataactccgccctgaagtctcggctgaccatcagcaaggacacatccaagaaccaggtggtgctgacaatgaccaatatggaccctgtggatacagccacctactattgcgccaagcactactattacggcggcagctatgccatggattactggggccagggcaccacagtgaccgtgagctccggaggcggcggctctggaggaggaggaagcggaggaggaggatccgagatcgtgctgacccagtcccctgccacactgagcctgtccccaggagagagggccaccctgtcttgtagagccagccaggatatctccaagtatctgaactggtaccagcagaagccaggacaggcaccaaggctgctgatctatcacacctctagactgcacagcggcatccccgacaggttctctggaagcggatccggcacagacttcaccctgaccatcagcaggctggagcccgaggacatcgccgtgtattactgccagcagggcaataccctgccttacacattcggccagggaaccaagctggagatcggaggaggaggatccggcggcggcggctccggcggcggcgggagcggcggcggcggctctgatatcgtgatgacccagtctccactgagcctgcctgtgacaccaggagagcctgcctctatcagctgtcggtctagccagtccctggtgcactctaacggcaatacctttctgcactggtatctgcagaagcctggccagagcccacagctgctgatctacaaggtgagcaacaggttctccggcgtgcctgaccgcttttccggctctggcagcggcaccgatttcacactgaagatctctagagtggaggccgaggacgtgggcgtgtattactgcatgcagagcacccacgtgctgacatttggcggcggcaccaagctggagatcaagagggcaggcggcggcggcagcggaggaggagggtctggcggcggcggctctcaggtgcagctggtgcagtccggagcagaggtgaagaagccaggagcctctgtgaaggtgagctgtaaggcctccggctatacattcaccgattacctgatgcactgggtgcgccaggcaccaggacagggactggagtggatgggctggatcaatacagagaccggcgagcccacctatgccgacgacttcaagggccgggtgacaatgaccagagacacatccacctctacagtgtacatggagctgtcctctctgcggagcgaggataccgccgtgtattactgtgcccccagcctgtattacggctattcctggtttacatattggggacagggcaccctggtgacagtgtccaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 46 - DNA, 2241 bp, Artificial sequence
  TanCAR 04 encoding nucleotide sequence
atggccctgccagtgaccgccctgctgctgccactggccctgctgctgcacgcagcaaggcctcaggtgacactgagggagagcggacccgccctggtgaagcctacccagacactgaccctgacatgcaccttcagcggcgtgtccctgccagactacggcgtgagctggatcaggcagccacctggcaaggccctggagtggctggccgtgatctggggctccgagaccacatactataactccgccctgaagtctcggctgaccatcagcaaggacacatccaagaaccaggtggtgctgacaatgaccaatatggaccctgtggatacagccacctactattgcgccaagcactactattacggcggcagctatgccatggattactggggccagggcactacagtgaccgtgagctccggcggcggcggctctggaggaggaggatccggaggaggaggatccgagatcgtgctgacccagtcccctgccacactgagcctgtccccaggagagagggccaccctgtcttgtagagccagccaggatatctccaagtatctgaactggtaccagcagaagccaggacaggcaccaaggctgctgatctatcacacctctagactgcacagcggcatccccgacaggttctctggaagcggatccggcacagacttcaccctgaccatcagcaggctggagcccgaggacatcgccgtgtattactgccagcagggcaataccctgccttacacattcggccagggaaccaagctggagatcgaggcagcagcaaaggaggcagcagccaaggaggccgccgccaaggatatcgtgatgacccagtctccactgagcctgccagtgacaccaggagagcctgcctctatcagctgtcggtctagccagtccctggtgcactctaacggcaatacctttctgcactggtatctgcagaagcctggccagagcccacagctgctgatctacaaggtgagcaacaggttctccggcgtgcctgaccgcttttccggctctggcagcggcaccgatttcacactgaagatctccagagtggaggccgaggacgtgggcgtgtattactgcatgcagtctacccacgtgctgacatttggcggcggcaccaagctggagatcaagagggccggaggaggaggatctggaggaggaggcagcggcggcggcggctcccaggtgcagctggtgcagtccggagcagaggtgaagaagccaggagcctctgtgaaggtgagctgtaaggcctccggctatacattcaccgattacctgatgcactgggtgcgccaggcaccaggacagggactggagtggatgggctggatcaatacagagaccggcgagcccacctatgccgacgacttcaagggccgggtgacaatgaccagagacacatccacctctacagtgtacatggagctgtcctctctgcggtctgaggataccgccgtgtattactgtgccccctctctgtattacggctatagctggtttacatattggggacagggcaccctggtgacagtgtccaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 47 - DNA, 2196 bp, Artificial sequence
  TanCAR 05 encoding nucleotide sequence
atggccctgccagtgaccgccctgctgctgccactggccctgctgctgcacgccgccagacccgacatcgtgatgacccagagcccactgtccctgcctgtgacaccaggagagcctgcaagcatctcctgcaggagctcccagtccctggtgcactctaacggcaataccttcctgcactggtatctgcagaagccaggccagagcccccagctgctgatctacaaggtgagcaaccggttctccggcgtgccagacagattttctggcagcggctccggcaccgatttcacactgaagatctccagagtggaggccgaggacgtgggcgtgtactattgcatgcagtctacccacgtgctgacatttggcggcggcaccaagctggagatcaagagggccggaggaggaggaagccaggtgcagctggtgcagtccggagcagaggtgaagaagccaggagcctctgtgaaggtgagctgtaaggcctccggctataccttcacagattacctgatgcactgggtgcgccaggcacctggacagggactggagtggatgggctggatcaataccgagacaggcgagccaacctatgccgacgacttcaagggccgggtgaccatgacaagagacacctctacaagcaccgtgtacatggagctgtctagcctgcggtccgaggataccgccgtgtactattgcgccccttccctgtactatggctattcttggttcacatactggggccagggcaccctggtgacagtgtctggaggaggaggaagcggaggaggaggaagcggcggcggcggctctggcggcggcggcagccaggtgaccctgagggagagcggccccgccctggtgaagcctacacagaccctgacactgacctgtacattttctggcgtgagcctgcccgattacggcgtgtcctggatcaggcagccacctggcaaggccctggagtggctggccgtgatctggggcagcgagaccacatactataactctgccctgaagagccggctgaccatctccaaggacacatctaagaaccaggtggtgctgaccatgacaaatatggaccctgtggataccgccacatactattgcgccaagcactactattacggcggctcctatgccatggattattggggacagggcaccacagtgaccgtgtcctctggaggaggaggatctgagatcgtgctgacccagagccctgccacactgtccctgtctccaggagagagggccacactgagctgtagggcatcccaggacatctctaagtatctgaactggtaccagcagaagcctggccaggccccaaggctgctgatctatcacaccagcaggctgcactccggcatccccgaccggttcagcggatccggatctggcacagacttcaccctgacaatcagccggctggagccagaggatatcgccgtgtattactgtcagcagggcaataccctgccctacacatttggccagggaactaagctggagatcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 48 - DNA, 2196 bp, Artificial sequence
  TanCAR 06 encoding nucleotide sequence
atggccctgccagtgaccgccctgctgctgccactggccctgctgctgcacgcagcaaggcctcaggtgacactgagggagagcggacccgccctggtgaagcctacccagacactgaccctgacatgcaccttcagcggcgtgtccctgccagactacggcgtgagctggatcaggcagccacctggcaaggccctggagtggctggccgtgatctggggctccgagaccacatactataactccgccctgaagtctcggctgaccatcagcaaggatacatccaagaaccaggtggtgctgacaatgaccaatatggaccccgtggatacagccacctactattgcgccaagcactactattacggcggctcttatgccatggactactggggccagggcactacagtgaccgtgagcagcggaggaggaggatccgatatcgtgatgacccagtctcctctgagcctgccagtgacaccaggagagcctgccagcatctcctgtaggtctagccagtctctggtgcacagcaacggcaatacctttctgcactggtatctgcagaagccaggccagagcccccagctgctgatctacaaggtgtccaataggttctctggcgtgccagaccgcttttctggcagcggctccggcaccgatttcacactgaagatctccagagtggaggccgaggacgtgggcgtgtattactgcatgcagtctacccacgtgctgacatttggcggcggcaccaagctggagatcaagagggccggaggaggaggatctggaggaggaggcagcggcggcggcggctccggcggcggcggctctcaggtgcagctggtgcagtccggagcagaggtgaagaagccaggagccagcgtgaaggtgtcctgtaaggcctctggctatacattcaccgattacctgatgcactgggtgcgccaggcaccaggacagggactggagtggatgggctggatcaacacagagaccggcgagcccacctatgccgacgacttcaagggccgggtgacaatgaccagagacacatctaccagcacagtgtacatggagctgtcctctctgcggagcgaggataccgccgtgtattactgcgccccttctctgtattacggctatagctggttcacatattggggacagggcaccctggtgacagtgagcggaggaggcggctccgagatcgtgctgacccagtcccctgccacactgtccctgtctccaggagagagggccaccctgagctgtagagcatcccaggacatctctaagtatctgaactggtaccagcagaagcctggacaggcaccaaggctgctgatctatcacaccagcagactgcactccggcatccccgacaggttcagcggatccggatctggcacagacttcaccctgacaatcagccggctggagcccgaggatatcgccgtgtattactgtcagcagggcaataccctgccttacacatttggccagggaactaagctggagatcaccacgacgccagcgccgcgaccaccaacaccggcgcccaccatcgcgtcgcagcccctgtccctgcgcccagaagcgtgccggccagcggcggggggcgcagtgcacacgagggggctggacttcgcctgtgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 49 - protein, 21 aa, Artificial sequence
  N-signal peptide amino acid sequence
MALPVTALLLPLALLLHAARP

SEQ ID NO: 50 - protein, 26 aa, Artificial sequence
  P2A amino acid sequence
RAKRGSGATNFSLLKQAGDVEENPGP

SEQ ID NO: 51 - protein, 1002 aa, Artificial sequence
  BiCAR amino acid sequence
MALPVTALLLPLALLLHAARPDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPMALPVTALLLPLALLLHAARPQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGGGGSGGGGSGGGGSEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 3006 bp, Artificial sequence
  BiCAR encoding nucleotide sequence
atggcgctgccagtgaccgcgcttctgctgccacttgcgctgctgctgcacgccgcgcgccccgacatcgtgatgacccagagccccctgtcgctgcctgtgactcccggggagccggccagcatttcgtgccggtcttctcaatcgctggttcacagtaacggcaacacgttccttcactggtacctgcagaagcccggccagtccccgcagctgctgatctacaaggtgtcgaaccgtttctcgggcgtccccgacaggtttagcggcagcggttctggtaccgatttcacccttaagatatcccgcgtggaggccgaggacgtgggagtatactactgtatgcagtccacccacgtactgaccttcggcggcggcacaaagctggagatcaaaagagctggaggcggtggctcgggcggtggcggttctgggggcgggggctctcaggtgcagctggtgcagtccggagctgaggtgaagaagcctggggcttccgtcaaggtgtcatgcaaggcttccggatacaccttcaccgactacctgatgcactgggtgcgccaggctccgggacagggactggagtggatgggctggattaatacagagaccggggagccaacctacgcggatgacttcaagggccgtgtcaccatgacgcgtgacacttctacttccaccgtgtacatggagctgtcctccctgcgctcggaagacactgccgtgtactactgtgctccttccctctattacggctacagctggttcacatattggggccagggtaccttggtcacggtcagtaccacgacccccgcaccccggcccccaacacctgctcctaccatcgcctcccagccgctctcgctgcgtccagaggcctgccgacccgcggccggcggcgccgttcataccagaggcttggatttcgcctgtgacatctatatctgggcacctctagctggcacctgcggggtgttgctactgtctctcgtgatcaccctgtactgtaagcgcggaaggaagaagttgctgtacatcttcaaacagcccttcatgcgtcccgtgcagaccacccaggaggaagacggatgctcatgccgattccccgaggaagaggagggcggctgcgagctgcgtgtaaaattttcccggtccgcggacgcacctgcctacaagcagggtcagaaccagttatacaacgagctcaacctgggccgcagggaggagtacgatgtgctggacaaacgccgcggacgcgacccggagatgggcggcaagccgcgcaggaagaatccgcaggaggggctgtacaacgagctacagaaggacaagatggccgaggcgtactccgagatcggtatgaaaggagagcgccgccgcggcaagggccacgacggcttgtatcaaggtctgtctactgctaccaaggacacctacgacgcccttcatatgcaggccttgccccctcgccgcgcgaagcgcggttctggcgccaccaacttctccctgctcaagcaggcaggtgatgtggaggagaatccagggccgatggccctccctgtcaccgccctgctgcttccgctggctcttctgctccacgccgctcggccccaggtcaccctgagagagagcggaccagccctggtgaaacctacacagaccctgaccctgacctgcacctttagcggagtgtccctgccagactacggagtgtcttggatccggcagccaccaggaaaagctctcgagtggctcgcagtgatttggggcagcgagaccacctactacaacagcgccctgaagagcaggctgaccatcagcaaggacaccagcaagaaccaggtggtgctgaccatgaccaacatggaccccgtggacaccgccacctactattgcgccaagcactactactacggcggcagctacgccatggactattggggacagggcaccaccgtgacagtgtctagcggaggaggaggaagcggaggaggaggatctggaggaggaggcagcgagattgtgctgacacagagcccagccacactgtctctgagcccaggagagagagccacactgtcttgcagggccagccaggacatcagcaagtacctgaattggtaccagcagaagcccggccaggctcctaggctgctgatctaccacaccagcaggctgcacagcggcatcccagacagattcagcggaagcggcagcggcacagacttcaccctgaccatcagcagactggagcccgaggacatcgccgtgtactattgccagcagggcaacaccctgccctacacctttggacagggcaccaagctggagatcaccactaccccagcaccgaggccacccaccccggctcctaccatcgcctcccagcctctgtccctgcgtccggaggcatgtagacccgcagctggtggggccgtgcatacccgtggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 53 - protein, 11 aa, Artificial sequence
  Murine-CD19 VL CDR2
HTSRLHSGVPS

SEQ ID NO: 54 - DNA, 15 bp, Artificial sequence
  Linker1 encoding nucleotide sequence
ggaggaggaggaagc

SEQ ID NO: 55 - DNA, 45 bp, Artificial sequence
  Linker2 encoding nucleotide sequence
ggaggaggaggaagcggaggaggaggaagcggaggaggaggaagc

SEQ ID NO: 56 - DNA, 60 bp, Artificial sequence
  Linker3 encoding nucleotide sequence
ggaggaggaggaagtggaggaggaggatccggcggcggcggctctggcggcggcggcagc

SEQ ID NO: 57 - DNA, 45 bp, Artificial sequence
  Linker4 encoding nucleotide sequence
gaggcagcagcaaaggaggcagcagccaaggaggcagcagcaaag

SEQ ID NO: 58 - DNA, 63 bp, Artificial sequence
  N-signal peptide encoding nucleotide sequence
atggccctccctgtcaccgccctgctgcttccgctggctcttctgctccacgccgctcggccc

SEQ ID NO: 59 - DNA, 357 bp, Artificial sequence
  H-BCMA VH  encoding nucleotide sequence
caggttcagctggttcagtctggcgccgaagtgaagaaacctggcgcctctgtgaaggtgtcctgcaaggccagcggctacacctttaccgactacctgatgcactgggtccgacaggctccaggacaaggcttggaatggatgggctggatcaacaccgagacaggcgagcctacctacgccgacgatttcaagggcagagtgaccatgaccagagacaccagcacctccaccgtgtacatggaactgagcagcctgagaagcgaggacaccgccgtgtactactgtgcccctagcctgtactacggctactcctggtttacctactggggccagggcaccctggtcacagtttct

SEQ ID NO: 60 - DNA, 339 bp, Artificial sequence
  H-BCMA VL encoding nucleotide sequence
gacatcgtgatgacacagagccctctgagcctgcctgtgacacctggcgaacctgccagcatcagctgtagaagcagccagagcctggtgcacagcaacggcaataccttcctgcactggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgtatgcagagcacccacgtgctgacctttggcggcggaacaaagctggaaatcaagcgggct

SEQ ID NO: 61 - DNA, 360 bp, Artificial sequence
  H-CD19 VH encoding nucleotide sequence
caggtcaccctgagagagagcggaccagccctggtgaaacctacacagaccctgaccctgacctgcacctttagcggagtgtccctgccagactacggagtgtcttggatccggcagccaccaggaaaagctctcgagtggctcgcagtgatttggggcagcgagaccacctactacaacagcgccctgaagagcaggctgaccatcagcaaggacaccagcaagaaccaggtggtgctgaccatgaccaacatggaccccgtggacaccgccacctactattgcgccaagcactactactacggcggcagctacgccatggactattggggacagggcaccaccgtgacagtgtctagc

SEQ ID NO: 62 - DNA, 318 bp, Artificial sequence
  H-CD19 VL encoding nucleotide sequence
gagattgtgctgacacagagcccagccacactgtctctgagcccaggagagagagccacactgtcttgcagggccagccaggacatcagcaagtacctgaattggtaccagcagaagcccggccaggctcctaggctgctgatctaccacaccagcaggctgcacagcggcatcccagacagattcagcggaagcggcagcggcacagacttcaccctgaccatcagcagactggagcccgaggacatcgccgtgtactattgccagcagggcaacaccctgccctacacctttggacagggcaccaagctggagatc

SEQ ID NO: 63 - DNA, 423 bp, Artificial sequence
  SFFV encoding nucleotide sequence
agctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggtacc

SEQ ID NO: 64 - protein, 755 aa, Artificial sequence
  TanCAR08 amino acid sequence
MALPVTALLLPLALLLHAARPDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSGGGGSGGGGSGGGGSGGGGSQVTLRESGPALVKPTQTLTLTCTFSGVSLPDYGVSWIRQPPGKALEWLAVIWGSETTYYNSALKSRLTISKDTSKNQVVLTMTNMDPVDTATYYCAKHYYYGGSYAMDYWGQGTTVTVSSGSTSGSGKPGSGEGSTKGEIVLTQSPATLSLSPGERATLSCRASQDISKYLNWYQQKPGQAPRLLIYHTSRLHSGIPDRFSGSGSGTDFTLTISRLEPEDIAVYYCQQGNTLPYTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - DNA, 2265 bp, Artificial sequence
  TanCAR08 encoding nucleotide sequence
atggcgttgcccgtgaccgctctcctgttgcctctagctctgctattacacgccgcgaggcccgacatcgtgatgacgcagagcccgctttcgttaccggtaacacccggcgaacctgcttccatttcgtgccgatcttctcaatcccttgtccactccaacggcaacacgttcctgcattggtacctgcagaagcctggtcagagccctcagctgttgatctacaaggtgagcaaccgcttctccggtgtccccgacaggtttagcggatccggatcagggaccgatttcaccctgaagatctctcgtgtggaggcagaggatgtgggcgtgtactactgtatgcaatccactcacgtgctgactttcggaggcggcaccaagctggagatcaagcgggccggcggtggaggctcgggcgggggtggttccgggggcggcggtagccaagtccagctggtgcagtccggcgcggaggtgaaaaaacctggcgcctccgtaaaggttagctgcaaggcctctggctacaccttcaccgactacctgatgcactgggtgagacaggctcccggccagggactggagtggatggggtggattaataccgagactggggaaccaacgtatgcggatgacttcaaaggccgtgtcactatgacccgcgacacttccaccagcaccgtgtacatggagctgtcatctctgaggtccgaggacaccgccgtgtattactgtgctccctctctttactacggttacagttggttcacctattggggccagggcacgctggttaccgtaagcggtgggggcggatccggcggcggcggctcaggcggcggtggctctggtggcggaggttcccaggtgaccctgcgcgagagcgggccagcactggtgaagccaacccagactctgaccctgacctgcactttctcaggcgtgtccttgcctgattacggcgtcagttggatccgccagcccccaggaaaagctctcgagtggctagcggtgatctggggctccgaaacgacctactacaacagcgccctcaagagtcgcctcacaatttccaaggacacctccaagaaccaggtggtgctgacgatgaccaacatggacccggtggacaccgctacatactattgtgcgaagcactactactacggcgggagctacgccatggactattggggacagggcaccaccgtcaccgtttcaagtgggtccaccagcggctccggcaaaccgggctccggggagggctccaccaagggagagattgtcctgacccagagcccggctactcttagtctgtcaccgggcgagcgcgccaccctgtcttgtagagcctctcaggacatctccaagtacttgaactggtaccagcagaagccgggacaggcgcctcgcttactgatttaccacacaagccgactccactccggcatccccgaccgcttctctgggtctggctcgggcactgatttcaccctgacgatctcccgcctggagccagaagatatcgccgtgtactactgtcaacagggcaatacgctgccctatacgtttgggcagggcactaagctcgagatcaccacaacccctgcgccgcgccctccgacccccgcccccaccatcgctagccagccgctgtctctgcgccccgaggcctgcaggcccgcggcgggaggtgctgtgcatacacgcgggctcgattttgcttgcgacatctatatatgggcacccctggcagggacctgcggggtgctgctgctgagcttggtgatcaccctttactgtaagcggggccgcaagaagttgctctacatcttcaagcagcccttcatgcgcccggtgcagacgacccaggaggaagacgggtgctcttgtcgttttcccgaagaggaggagggcggttgcgagctgcgcgtcaaattttctcgctcggccgacgcccctgcctatcagcagggccagaaccagttgtacaacgagcttaacctgggtcggcgtgaggagtacgacgtgctggacaagcgccgtggtcgtgatccagagatgggcggcaagccccgtcgcaagaatccacaggaggggctttacaacgagctgcagaaggacaaaatggctgaggcctactccgagatcggaatgaagggtgagcggcggcgcggcaagggccatgacggcctttaccagggcctctcgactgccactaaagacacttacgacgctctgcacatgcaggccctgccccctcgc

SEQ ID NO: 66 - protein, 726 aa, Artificial sequence
  TanCAR10 amino acid sequence
MALPVTALLLPLALLLHAARPDIVMTQSPLSLPVTPGEPASISCRSSQSLVHSNGNTFLHWYLQKPGQSPQLLIYKVSNRFSGVPDRFSGSGSGTDFTLKISRVEAEDVGVYYCMQSTHVLTFGGGTKLEIKRAGGGGSGGGGSGGGGSQVQLVQSGAEVKKPGASVKVSCKASGYTFTDYLMHWVRQAPGQGLEWMGWINTETGEPTYADDFKGRVTMTRDTSTSTVYMELSSLRSEDTAVYYCAPSLYYGYSWFTYWGQGTLVTVSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 2178 bp, Artificial sequence
  TanCAR10 encoding nucleotide sequence
atggccctgcccgtcaccgccctattactgccgctggcgctcctgctgcacgcggcgcggccggatatcgtgatgacccagagccccctctccctaccggtgacacctggggagcctgcgtcgatctcctgtcgctcgtcccaatctttggtgcactcaaatggcaacacgttcctccattggtacttgcagaagcccgggcagagccctcagctgctcatctacaaggttagcaaccgcttttctggcgtgcctgaccgcttcagtggatcgggatcaggaaccgacttcactctgaagatatccagagtagaggccgaggatgtgggcgtgtactactgtatgcagtctacgcatgtactgaccttcggcgggggcactaagctggagatcaagcgcgccggcggcggcggttcggggggtggtggttctgggggcggcggctcccaggtccagttggtgcagagcggggctgaggttaagaagccgggcgcttctgtcaaggtgtcctgcaaagcaagcggctacaccttcaccgactacctcatgcactgggtcagacaggcccccggacaggggctggaatggatgggatggattaacacggagaccggcgagccaacctacgcggacgatttcaaggggcgggtaacgatgacccgcgacacttccacttctaccgtgtacatggagctgtcctccctgcgctctgaagatactgccgtgtactactgtgctccttcactttattatggctattcttggtttacgtattggggccagggaaccctggtcaccgtttccggaggcggggggtccggaggaggcggctctggtgggggaggtagcggcggtggcggatcggaggtgaagctgcaagagtctgggcccggtctagtggctcccagtcagtcgcttagcgtcacctgcacagtgtcaggcgtgtccctgcccgactacggcgtttcctggatccgccagccacccaggaagggtttggagtggctgggtgtgatctggggctccgagactacttactacaactccgctctgaagtcccgtctgacaattatcaaagacaactccaagtcgcaggtgttcctgaaaatgaacagcctgcagacagatgacactgccatctattactgtgccaagcactactactacggcggtagctacgccatggattattggggccagggcacctccgtgaccgtgagtagcgggtctacttccggatcaggtaagcccggctcgggtgagggctccacgaagggtgatatccagatgacccagactaccagctccttgtctgcatcgctgggcgacagggtgaccatcagctgccgagctagccaggacatctccaagtacctcaactggtaccagcagaagcccgacggcaccgtcaagcttcttatttaccataccagccgcctgcacagtggtgtcccgtcccgcttctccggcagcgggtccggaaccgactactctctgaccatctccaacctggaacaggaggacatcgccacctatttctgtcagcagggtaatacattaccctataccttcggtggcggcactaaactggagatcaccgagagtaagtacggccctccctgcccaccctgcccattttgggtcctggtggtggtcggtggcgttttggcctgttacagcttgctggtgaccgtggcattcattattttttgggtcaagcgcggccgcaaaaagctgttatacatcttcaaacagccgttcatgcgtccggtgcagacgacccaggaggaggacggctgctcttgccggttccccgaggaggaggagggcggctgcgaactgcgcgtgaaattttctcgcagcgccgacgccccggcctacaagcagggccagaaccagctgtacaacgagttgaacctgggccgtcgggaagagtacgacgtgctggacaaaagacgcggtcgcgaccctgagatgggcggaaagcctcgccgaaagaatccacaggagggcctctacaacgaactccagaaggacaagatggcggaggcatacagcgagatcggtatgaaaggcgagcgccgtcgtgggaagggccacgacggcctgtaccaaggcctgtccaccgctaccaaggacacctacgatgcgctccacatgcaggctcttcctccgagg

SEQ ID NO: 68 - protein, 18 aa, Artificial sequence
  Linker 5 amino acid sequence
GSTSGSGKPGSGEGSTKG

SEQ ID NO: 69 - DNA, 54 bp, Artificial sequence
  Linker5  encoding nucleotide sequence
gggtctacttccggatcaggtaagcccggctcgggtgagggctccacgaagggt

SEQ ID NO: 70 - protein, 12 aa, Artificial sequence
  IgG4Hinge amino acid sequence
ESKYGPPCPPCP

SEQ ID NO: 71 - DNA, 36 bp, Artificial sequence
  IgG4Hinge encoding nucleotide sequence
gagagtaagtacggccctccctgcccaccctgccca

SEQ ID NO: 72 - protein, 27 aa, Artificial sequence
  CD28TM amino acid sequence
FWVLVVVGGVLACYSLLVTVAFIIFWV

SEQ ID NO: 73 - DNA, 81 bp, Artificial sequence
  CD28TM encoding nucleotide sequence
ttttgggtcctggtggtggtcggtggcgttttggcctgttacagcttgctggtgaccgtggcattcattattttttgggtc

SEQ ID NO: 74 - protein, 112 aa, Artificial sequence
  CD3(Zeta) intracellular signaling domain amino acid sequence-2
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 336 bp, Artificial sequence
  CD3(Zeta) intracellular signaling domain encoding nucleotide sequence-2
cgcgtcaaattttctcgctcggccgacgcccctgcctatcagcagggccagaaccagttgtacaacgagcttaacctgggtcggcgtgaggagtacgacgtgctggacaagcgccgtggtcgtgatccagagatgggcggcaagccccgtcgcaagaatccacaggaggggctttacaacgagctgcagaaggacaaaatggctgaggcctactccgagatcggaatgaagggtgagcggcggcgcggcaagggccatgacggcctttaccagggcctctcgactgccactaaagacacttacgacgctctgcacatgcaggccctgccccctcgc

SEQ ID NO: 76 - protein, 120 aa, Artificial sequence
  Murine-CD19 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 77 - protein, 107 aa, Artificial sequence
  Murine-CD19 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 78 - DNA, 360 bp, Artificial sequence
  Murine-CD19 VH encoding nucleotide sequence
gaggtgaagctgcaagagtctgggcccggtctagtggctcccagtcagtcgcttagcgtcacctgcacagtgtcaggcgtgtccctgcccgactacggcgtttcctggatccgccagccacccaggaagggtttggagtggctgggtgtgatctggggctccgagactacttactacaactccgctctgaagtcccgtctgacaattatcaaagacaactccaagtcgcaggtgttcctgaaaatgaacagcctgcagacagatgacactgccatctattactgtgccaagcactactactacggcggtagctacgccatggattattggggccagggcacctccgtgaccgtgagtagc

SEQ ID NO: 79 - DNA, 321 bp, Artificial sequence
  Murine-CD19 VL encoding nucleotide sequence
gatatccagatgacccagactaccagctccttgtctgcatcgctgggcgacagggtgaccatcagctgccgagctagccaggacatctccaagtacctcaactggtaccagcagaagcccgacggcaccgtcaagcttcttatttaccataccagccgcctgcacagtggtgtcccgtcccgcttctccggcagcgggtccggaaccgactactctctgaccatctccaacctggaacaggaggacatcgccacctatttctgtcagcagggtaatacattaccctataccttcggtggcggcactaaactggagatcacc

SEQ ID NO: 80 - protein, 486 aa, Artificial sequence
  FMC63 CAR full-length amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - DNA, 1458 bp, Artificial sequence
  FMC63 CAR encoding nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg